(19) United States
(12) Patent Application Publication
(10) Pub. No.: US 2011/0224186 A1
(43) Pub. Date: Sep. 15, 2011

(54) CARBACEPHEM BETA-LACTAM ANTIBIOTICS

(75) Inventors: Allan Scott Wagman, Belmont, CA (US); Heinz Ernst Moser, San Mateo, CA (US)

(73) Assignee: Achaogen, Inc., South San Francisco, CA (US)

(21) Appl. No.: 13/044,800

(22) Filed: Mar. 10, 2011

Related U.S. Application Data
(63) Continuation of application No. PCT/US2009/056554, filed on Sep. 10, 2009.
(60) Provisional application No. 61/171,678, filed on Apr. 22, 2009.
(60) Provisional application No. 61/095,827, filed on Sep. 10, 2008.

(51) Int. Cl. A61K 31/397; C07D 463/22; A61P 31/04

(52) U.S. Cl. 514/210.04; 540/205

(57) ABSTRACT

Carbacephem β-lactam antibiotics having the following chemical structure (I) are disclosed:

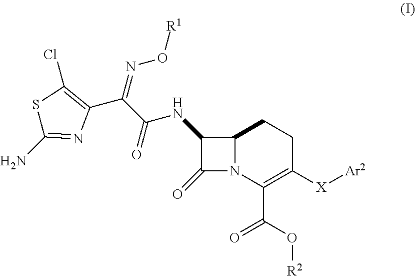

including stereoisomers, pharmaceutically acceptable salts, esters and prodrugs thereof, wherein Ar², X, R¹ and R² are as defined herein. The compounds are useful for the treatment of bacterial infections, in particular those caused by methicillin-resistant Staphylococcus spp.

### CROSS-REFERENCE TO RELATED APPLICATION

This application is a continuation of International PCT Patent Application No. PCT/US2009/056554, which was filed on Sep. 10, 2009, now pending, which claims the benefit under 35 U.S.C. §119(e) of U.S. Provisional Patent Application No. 61/095,827, filed Sep. 10, 2008, and U.S. Provisional Patent Application No. 61/171,678, filed Apr. 22, 2009, which applications are incorporated herein by reference in their entireties.

### BACKGROUND

1. Field

The present invention relates to novel carbacephem β-lactam antibiotics, and the use of such compounds to treat bacterial infections, in particular, infections caused by bacterial species resistant to conventional β-lactams.

2. Description of the Related Art

Over the past three decades a variety of antibiotics have become available for clinical use. One class of antibiotics that has seen remarkable growth is the β-lactams, over 70 of which have entered clinical use since 1965. Unfortunately, the widespread use of these antibiotics has resulted in an alarming increase in the number of resistant strains, especially among clinically important bacteria such as the genera Salmonella, Enterobacteriaceae, Pseudomonas and Staphylococcus.

Bacterial resistance to cephalosporins occurs primarily through three mechanisms: (a) destruction of the antibiotic by [3-lactamases; (b) decreased penetration due to changes in bacterial outer membrane composition; and (c) alteration of penicillin-binding proteins (PBPs) resulting in interference with βlactam binding. The latter pathway is especially important, as the binding of β-lactams to PBPs is essential for inhibiting peptidoglycan biosynthesis (peptidoglycan is a required bacterial cell-wall component). Certain Gram-positive bacteria such as methicillin-resistant Staphylococcus aureus (“MRSA”) and various genus Enterococcus bacteria are highly resistant to β-lactam antibiotics. The resistance of MRSA is due to the presence of a PBP called PBP2a, which binds very poorly to β-lactam antibiotics. The options for treating infections caused by MRSA are limited and there is a need for new antibiotics with activity against these strains.

In recent years, a novel family of β-lactam antibiotics, the carbacephems (1), has been sporadically touted as having promise against MRSAs and other resistant species. In compound (1), R₁ and R₂ are generally described as aromatic and heteroaromatic entities, and R₃ has generally been reported as an optionally substituted alkyl group.

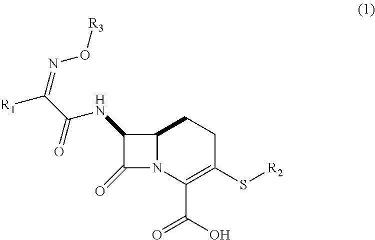

However, one problem with the carbacephem compounds developed thus far is that researchers investigating the family have been unable to achieve an acceptable balance between MRSA potency and serum protein binding. That is, MRSA activity was demonstrated relatively early on to correlate with lipophilicity; the more lipophilic the carbacephem, the greater its potency. Unfortunately, the greater the lipophilicity of the compound, the greater is its tendency toward high protein binding. Protein binding reduces the concentration of free drug circulating in blood. Lower circulating free drug concentrations typically result in less efficacious beta-lactams. Lack of oral bioavailability is another issue facing MRSA active beta-lactams. Historically, cephalosporins were both poorly absorbed by oral dosing and suffered from hydrolytic degradation, due to chemical instability, in the acidic environment of the stomach. Carbacephems offer an advantage for treating community-acquired MRSA which is most conveniently treated by oral antibiotics. Since carbacephems, due to their molecular structure, are intrinsically more stable to the gastric environment, this class of beta-lactam has a much greater potential for development as an oral agent.

Despite the above, carbacephems remain an intriguing approach to dealing with MRSA and other resistant bacterial species. What is needed, however, is a novel class of carbacephems that achieves the requisite balance of MRSA potency, protein binding and oral availability. The present invention addresses this need and provides further related advantages.

### BRIEF SUMMARY

In brief, the present invention is directed to novel carbacephem β-lactam antibiotics, including stereoisomers, pharmaceutically acceptable salts, esters and prodrugs thereof, and the use of such compounds to treat bacterial infections, in particular, infections caused by bacterial species resistant to conventional β-lactams, such as MRSA.

In one embodiment, a compound is provided having the following structure (I):

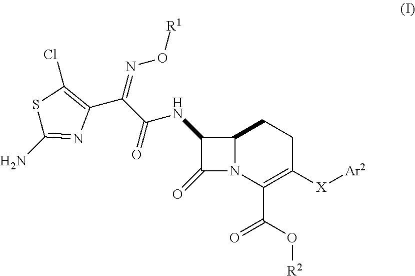

or a stereoisomer, pharmaceutically acceptable salt, ester, or prodrug thereof,

wherein:

R¹ is selected from hydrogen, optionally substituted alkyl, optionally substituted alkenyl, optionally substituted alkynyl, optionally substituted alkoxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl and —C(═O)R^{1a},

wherein:
R^{1a} is selected from hydrogen, optionally substituted alkyl, optionally substituted alkenyl, optionally substituted alkynyl, optionally substituted alkoxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl and optionally substituted heteroarylalkyl;

R² is selected from hydrogen, optionally substituted alkyl, optionally substituted alkenyl, optionally substituted alkynyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl and optionally substituted heteroarylalkyl;

X is selected from —O—, —C(═O)—, —SCH₂—, —CH₂S—, —SCH═CH—, —CH═CHS—, —SCH₂S—, —OCH₂—, —CH₂O—, optionally substituted alkylene, optionally substituted alkenylene and optionally substituted cycloalkyl; and

Ar² is optionally substituted aryl or optionally substituted heteroaryl.

In another embodiment, a pharmaceutical composition is provided comprising a compound having structure (I), or a stereoisomer, pharmaceutically acceptable salt, ester or prodrug thereof, and a pharmaceutically acceptable carrier, diluent or excipient.

In another embodiment, a method of using a compound having structure (I) in therapy is provided. In particular, the present invention provides a method of treating a bacterial infection is provided comprising administering a pharmaceutically effective amount of a compound having structure (I), or a stereoisomer, pharmaceutically acceptable salt, ester or prodrug thereof, to a mammal in need thereof. In certain embodiments, the bacterial infection may be caused by a β-lactam antibiotic-resistant bacterium, such as a methicillin-resistant genus Staphylococcus bacterium.

These and other aspects of the invention will be evident upon reference to the following detailed description.

### DETAILED DESCRIPTION

In the following description, certain specific details are set forth in order to provide a thorough understanding of various embodiments of the invention. However, one skilled in the art will understand that the invention may be practiced without these details.

Unless the context requires otherwise, throughout the specification and claims which follow, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in the specification and appended claims, unless specified to the contrary, the following terms have the meaning indicated:

“Amino” refers to the —NH₂ radical.

“Cyano” refers to the —CN radical.

“Hydroxy” or “hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Alkyl” refers to a straight or branched hydrocarbon chain radical consisting solely of carbon and hydrogen atoms, containing no unsaturation, having from one to twelve carbon atoms (C₁-C₁₂ alkyl), preferably one to eight carbon atoms (C₁-C₈ alkyl) or one to six carbon atoms (C₁-C₆ alkyl), and which is attached to the rest of the molecule by a single bond, e.g., methyl, ethyl, n-propyl, 1-methylethyl(iso-propyl), n-butyl, n-pentyl, 1,1-dimethylethyl(t-butyl), 3-methylhexyl, 2-methylhexyl, and the like. Unless stated otherwise specifically in the specification, an alkyl group may be optionally substituted.

“Alkenyl” refers to a straight or branched hydrocarbon chain radical group consisting solely of carbon and hydrogen atoms, containing at least one double bond, having from two to twelve carbon atoms, preferably two to eight carbon atoms and which is attached to the rest of the molecule by a single bond, e.g., ethenyl, prop-1-enyl, but-1-enyl, pent-1-enyl, penta-1,4-dienyl, and the like. Unless stated otherwise specifically in the specification, an alkenyl group may be optionally substituted.

“Alkynyl” refers to a straight or branched hydrocarbon chain radical group comprising solely of carbon and hydrogen atoms, containing at least one triple bond, optionally containing at least one double bond, having from two to twelve carbon atoms, preferably two to eight carbon atoms and which is attached to the rest of the molecule by a single bond, for example, ethynyl, propynyl, butynyl, pentynyl, hexynyl, and the like. Unless stated otherwise specifically in the specification, an alkynyl group may be optionally substituted.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain may be optionally substituted.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, e.g., ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain may be optionally substituted.

“Alkynylene” or “alkynylene chain” refers to a straight or branched divalent hydrocarbon chain linking the rest of the molecule to a radical group, consisting solely of carbon and hydrogen, containing at least one triple bond and having from two to twelve carbon atoms, e.g., propynylene, n-butynylene, and the like. The alkynylene chain is attached to the rest of the molecule through a single bond and to the radical group through a double bond or a single bond. The points of attachment of the alkynylene chain to the rest of the molecule and to the radical group can be through one carbon or any two carbons within the chain. Unless stated otherwise specifically in the specification, an alkynylene chain may be optionally substituted.

“Alkoxy” refers to a radical of the formula —ORₐ where Rₐ is an alkyl radical as defined above containing one to twelve carbon atoms. Unless stated otherwise specifically in the specification, an alkoxy group may be optionally substituted.

“Alkoxyalkyl” refers to a radical of the formula —R_{b}—O—R, where R_{b} is an alkylene chain as defined above and Rₐ is an alkyl radical as defined above. The oxygen atom may be bonded to any carbon in the alkylene chain and in the alkyl radical. Unless stated otherwise specifically in the specification, an alkoxyalkyl group may be optionally substituted.

“Aryl” refers to a hydrocarbon ring system radical comprising hydrogen, 6 to 18 carbon atoms and at least one aromatic ring. For purposes of this invention, the aryl radical may be a monocyclic, bicyclic, tricyclic or tetracyclic ring system, which may included fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Aralkyl” refers to a radical of the formula —R_{b}—R_{c} where R_{b} is an alkylene chain as defined above and R_{c} is one or more aryl radicals as defined above, for example, benzyl, diphenylmethyl and the like. Unless stated otherwise specifically in the specification, an aralkyl group may be optionally substituted.

“Aralkenyl” refers to a radical of the formula —R_{d}—R_{c} where R_{d} is an alkenylene chain as defined above and R_{c} is one or more aryl radicals as defined above. Unless stated otherwise specifically in the specification, an aralkenyl group may be optionally substituted.

“Aralkynyl” refers to a radical of the formula —RₑR_{c} where Rₑ is an alkynylene chain as defined above and R_{c} is one or more aryl radicals as defined above. Unless stated otherwise specifically in the specification, an aralkynyl group may be optionally substituted.

“Aryloxy” refers to a radical of the formula —OR_{b} where R_{b} is an aryl group as defined above. Unless stated otherwise specifically in the specification, an aryloxy group may be optionally substituted.

“Aralkyloxy” refers to a radical of the formula —OR_{b} where R_{b} is an aralkyl group as defined above. Unless stated otherwise specifically in the specification, an aralkyloxy group may be optionally substituted.

“Cycloalkyl” or “carbocyclic ring” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptly, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group may be optionally substituted.

“Cycloalkylalkyl” refers to a radical of the formula —R_{b}R_{g} where R_{b} is an alkylene chain as defined above and R_{g} is a cycloalkyl radical as defined above. Unless stated otherwise specifically in the specification, a cycloalkylalkyl group may be optionally substituted.

“Cycloalkylalkenyl” refers to a radical of the formula —R_{d}R_{g} where R_{d} is an alkenylene chain as defined above and R_{g} is a cycloalkyl radical as defined above. Unless stated otherwise specifically in the specification, a cycloalkylalkenyl group may be optionally substituted.

“Cycloalkylalkynyl” refers to a radical of the formula —RₑR_{g} where Rₑ is an alkynylene radical as defined above and R_{g} is a cycloalkyl radical as defined above. Unless stated otherwise specifically in the specification, a cycloalkylalkynyl group may be optionally substituted.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. Unless stated otherwise specifically in the specification, a haloalkyl group may be optionally substituted.

“Haloalkenyl” refers to an alkenyl radical, as defined above, that is substituted by one or more halo radicals, as defined above. Unless stated otherwise specifically in the specification, a haloalkenyl group may be optionally substituted.

“Haloalkynyl” refers to an alkynyl radical, as defined above, that is substituted by one or more halo radicals, as defined above. Unless stated otherwise specifically in the specification, a haloalkynyl group may be optionally substituted.

“Heterocyclyl” or “heterocyclic ring” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, Unless stated otherwise specifically in the specification, a heterocyclyl group may be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group may be optionally substituted.

“Heterocyclylalkyl” refers to a radical of the formula —R_{b}Rₕ where R_{b} is an alkylene chain as defined above and Rₕ is a heterocyclyl radical as defined above, and if the heterocyclyl is a nitrogen-containing heterocyclyl, the heterocyclyl may be attached to the alkyl radical at the nitrogen atom. Unless stated otherwise specifically in the specification, a heterocyclylalkyl group may be optionally substituted.

“Heterocyclylalkenyl” refers to a radical of the formula —R_{d}Rₕ where R_{d} is an alkenylene chain as defined above and Rₕ is a heterocyclyl radical as defined above, and if the heterocyclyl is a nitrogen-containing heterocyclyl, the heterocyclyl may be attached to the alkenylene chain at the nitrogen atom. Unless stated otherwise specifically in the specification, a heterocyclylalkenyl group may be optionally substituted.

“Heterocyclylalkynyl” refers to a radical of the formula —RₑRₕ where Rₑ is an alkynylene chain as defined above and Rₕ is a heterocyclyl radical as defined above, and if the heterocyclyl is a nitrogen-containing heterocyclyl, the heterocyclyl may be attached to the alkynyl radical at the nitrogen atom. Unless stated otherwise specifically in the specification, a heterocyclylalkynyl group may be optionally substituted.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl(benzothiophenyl), benzotriazolyl, benzo[4,6]imidazo pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrrolyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group may be optionally substituted.

“Heteroarylalkyl” refers to a radical of the formula —R_{b}Rᵢ where R_{b} is an alkylene chain as defined above and Rᵢ is a heteroaryl radical as defined above. Unless stated otherwise specifically in the specification, a heteroarylalkyl group may be optionally substituted.

“Heteroarylalkenyl” refers to a radical of the formula —R_{d}Rᵢ where R_{d} is an alkenylene chain as defined above and Rᵢ is a heteroaryl radical as defined above. Unless stated otherwise specifically in the specification, a heteroarylalkenyl group may be optionally substituted.

“Heteroarylalkynyl” refers to a radical of the formula —RₑRᵢ where Rₑ is an alkynylene chain as defined above and Rᵢ is a heteroaryl radical as defined above. Unless stated otherwise specifically in the specification, a heteroarylalkynyl group may be optionally substituted.

“Hydroxyalkyl” refers to an alkyl radical, as defined above, substituted by one or more hydroxy groups.

The term “substituted” used herein means any of the above groups alkyl, alkenyl, alkynyl, alkylene, alkenylene, alkynylene, alkoxy, alkoxyalkyl, aryl, aralkyl, aralkenyl, aralkynyl, aryloxy, aralkyloxy, cycloalkyl, cycloalkylalkyl, cycloalkylalkenyl, cycloalkylalkynyl, haloalkyl, haloalkenyl, haloalkynyl, heterocyclyl, N-heterocyclyl, heterocyclylalkyl, heterocyclylalkenyl, heterocyclylalkynyl, heteroaryl, N-heteroaryl, heteroarylalkyl, heteroarylalkenyl and/or heteroarylalkynyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more bonds are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. “Substituted” further means any of the above groups in which one or more bonds are replaced by a bond to an amino, cyano, hydroxyl, imino, nitro, oxo, thioxo, halo, hydroxyalkyl, alkyl, alkenyl, alkynyl, alkylene, alkenylene, alkynylene, alkoxy, alkoxyalkyl, aryl, aralkyl, aralkenyl, aralkynyl, aryloxy, aralkyloxy, cycloalkyl, cycloalkylalkyl, cycloalkylalkenyl, cycloalkylalkynyl, haloalkyl, haloalkenyl, haloalkynyl, heterocyclyl, N-heterocyclyl, heterocyclylalkyl, heterocyclylalkenyl, heterocyclylalkynyl, heteroaryl, N-heteroaryl, heteroarylalkyl, heteroarylalkenyl and/or heteroarylalkynyl group. In addition, the foregoing substituents may also be optionally substituted with one or more of the above substituents.

“Prodrug” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987.

The term “prodrug” is also meant to include any covalently bonded carriers, which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the invention and the like. More specifically, example of prodrugs include (in addition to the prodrugs of structures (II) and (III) described below), but are not limited to, compounds of structure (I) wherein R¹ is alkyl (such as, for example, methyl) and R¹ is bonded to an ester group (such as, for example, —OC(═O)CH₃ or —OC(═O)C(CH₃)₂).

The invention disclosed herein is also meant to encompass all pharmaceutically acceptable compounds of a structure disclosed herein being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵, respectively. These radiolabelled compounds could be useful to help determine or measure the effectiveness of the compounds, by characterizing, for example, the site or mode of action on the sodium channels, or binding affinity to pharmacologically important site of action on the sodium channels. Certain isotopically-labelled compounds of a structure disclosed herein, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of a structure disclosed herein can generally be prepared by conventional techniques known to those skilled in the art or by processes analogous to those as set out below using an appropriate isotopically-labeled reagent in place of the non-labeled reagent previously employed.

The invention disclosed herein is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the invention includes compounds produced by a process comprising contacting a compound of this invention with a mammal for a period of time sufficient to yield a metabolic product thereof. Such products are typically are identified by administering a radiolabelled compound of the invention in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets (e.g., cats, dogs, swine, cattle, sheep, goats, horses, rabbits), and non-domestic animals such as wildlife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecyl sulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefore.

“Effective amount” or “therapeutically effective amount” refers to that amount of a compound of the invention which, when administered to a mammal, preferably a human, is sufficient to effect treatment, as defined below, of a bacterial infection in the mammal, preferably a human. The amount of a compound of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:

(i) preventing the disease or condition from occurring in a mammal, in particular, when such mammal is predisposed to the condition but has not yet been diagnosed as having it;

(ii) inhibiting the disease or condition, i.e., arresting its development;

(iii) relieving the disease or condition, i.e., causing regression of the disease or condition; or

(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the invention, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (-), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

“MIC”, which stands for minimum inhibitory concentration, refers to that concentration, in μg/mL, of a compound of this invention that inhibits the growth and/or proliferation of a strain of bacteria by at least 80% compared to an untreated control.

“MRSA” refers to methicillin-resistant Staphylococcus aureus.

“Bacterial infection” refers to the establishment of a sufficient population of a pathogenic bacteria in a patient to have a deleterious effect on the health and well-being of the patient and/or to give rise to discernable symptoms associated with the particular bacteria.

“β-lactam resistant bacterium” or “β-lactam antibiotic resistant bacterium” refers to bacterium against which a known β-lactam antibiotic, such as methicillin and ampicillin, has a minimum inhibitory concentration (MIC) greater than 8 μg/mL.

As noted above, in one embodiment of the present invention, compounds having antibacterial activity are provided, the compounds having the following structure (I):

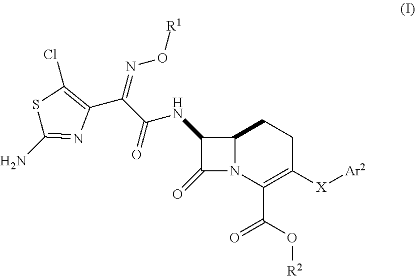

or a stereoisomer, pharmaceutically acceptable salt, ester, or prodrug thereof,

wherein:

R¹ is selected from hydrogen, optionally substituted alkyl, optionally substituted alkenyl, optionally substituted alkynyl, optionally substituted alkoxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl and —C(═O)R^{1a},
wherein:
R^{1a} is selected from hydrogen, optionally substituted alkyl, optionally substituted alkenyl, optionally substituted alkynyl, optionally substituted alkoxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl and optionally substituted heteroarylalkyl;

R² is selected from hydrogen, optionally substituted alkyl, optionally substituted alkenyl, optionally substituted alkynyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl and optionally substituted heteroarylalkyl;

X is selected from —O—, —C(═O)—, —SCH₂—, —CH₂S—, —SCH═CH—, —CH═CHS—, —SCH₂S—, —OCH₂—, —CH₂O—, optionally substituted alkylene, optionally substituted alkenylene and optionally substituted cycloalkyl; and

Ar² is optionally substituted aryl or optionally substituted heteroaryl.

In further embodiments, R¹ is hydrogen.

In other further embodiments, R¹ is alkyl and is selected from methyl, ethyl, n-propyl, iso-propyl, n-butyl, tert-butyl, iso-butyl and sec-butyl.

In other further embodiments, R¹ is substituted alkyl and is optionally substituted haloalkyl. For example, in certain embodiments, R¹ is selected from —CH₂CH₂Cl, —CH₂CH₂F, —CH₂CHF₂, —CH₂CF₃, —CHFCH₂F, —CHFCHF₂, —CHFCF₃, —CH₂F, —CHF₂, —CF₃ and —CH₂CH₂CH₂F.

In other further embodiments, R¹ is substituted alkyl and is optionally substituted alkoxyalkyl or optionally substituted hydroxyalkyl. For example, in certain embodiments, R¹ is —CH₂CH₂OH, —CH₂CH₂OMe or —CH₂CH₂OCF₃.

In other further embodiments, R¹ is substituted alkyl and is —CH₂CH₂SMe, —CH₂CH₂SO₂Me, —CH₂CH₂NMe₃, —CH₂CH₂NMe₂ or —CH₂CN.

In other further embodiments, R¹ is alkenyl and is —CH₂CH═CH₂.

In other further embodiments, R¹ is substituted alkenyl and is optionally substituted haloalkenyl. For example, in certain embodiments, R¹ is —CH₂CH═CCl₂ or —CH₂CH═CF₂.

In other further embodiments, R¹ is cycloalkyl and is selected from cyclopropyl, cyclobutyl, cyclopentyl, cyclopentenyl, cyclohexyl, and cyclohexenyl.

In other further embodiments, R² is hydrogen.

In other further embodiments, R² is alkyl and is selected from methyl, ethyl, n-propyl, iso-propyl, n-butyl, tert-butyl, iso-butyl and sec-butyl.

In other further embodiments, R² is substituted alkyl and is selected from haloalkyl, —(CH₂)ₙ—R^{2a}, —(CH₂)ₙN(R^{2a})₂, —(CH₂)ₙN(R^{2a})₃, —(CH₂)ₙSOR^{2a}, —(CH₂)ₙSO₂R^{2a} and —(CH₂)ₙCN; n is 1 or 2; and each R^{2a} is independently optionally substituted alkyl. For example, in certain embodiments, R² is selected from —CH₂F, —CH₂CN, —CH₂CH₂F, —CH₂CHF₂, —CH₂CF₃, —CH₂CH₂OCH₃, —CH₂CH₂OCF₃, —CH₂CH₂SO₂CH₃, —CH₂CH₂CN, —CH₂CH₂N(CH₃)₃ and —CH₂CH₂N(CH₃)₂.

In other further embodiments, R² is cycloalkyl and is selected from cyclopropyl, cyclobutyl, cyclopentyl and cyclohexyl.

In other further embodiments, R² is aryl and is phenyl.

In other further embodiments, R² is substituted aryl and is substituted phenyl.

In other further embodiments, R² is heteroaryl and is a 6-membered ring comprising at least one N atom.

In other further embodiments, the compound is a pharmaceutically acceptable salt of structure (I) having the following structure (II):

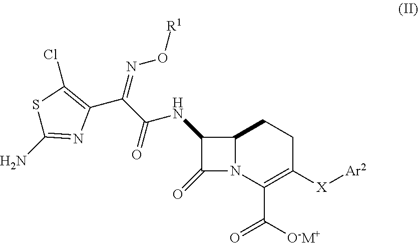

wherein M is an alkali metal atom.

In other further embodiments, the compound is a prodrug of structure (I) having the following structure (III):

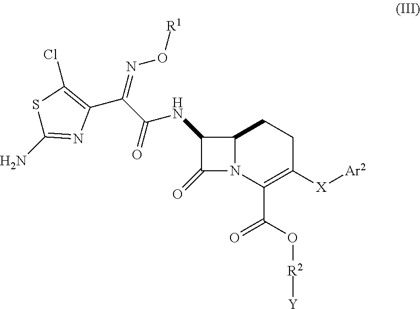

wherein R² and Y, taken together, are selected from:

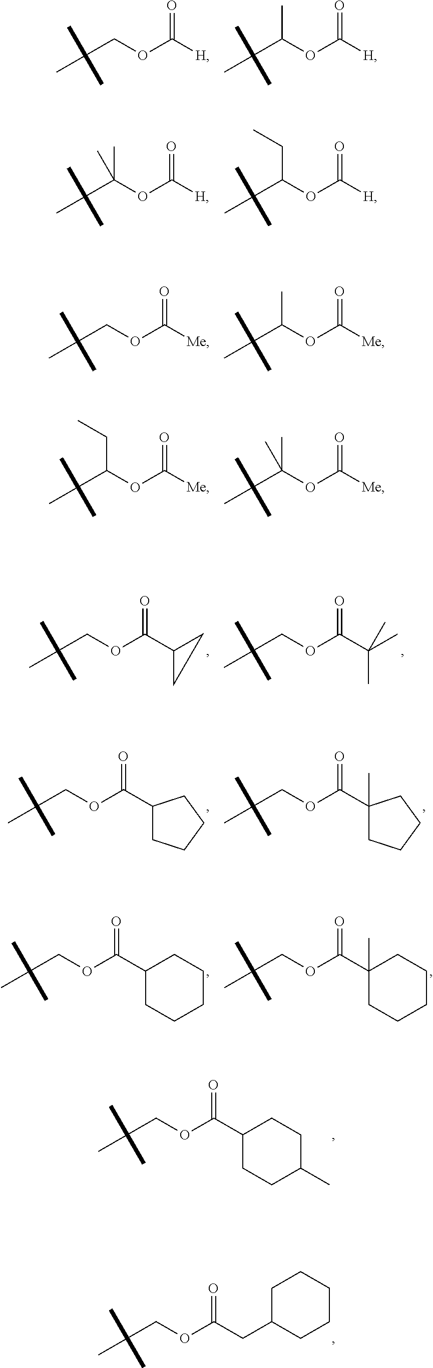

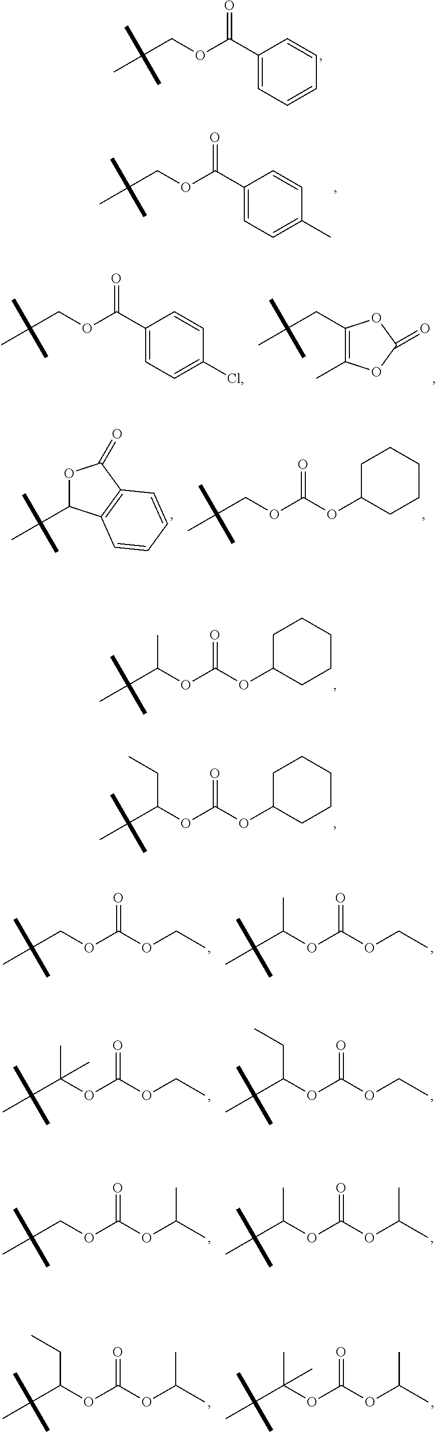

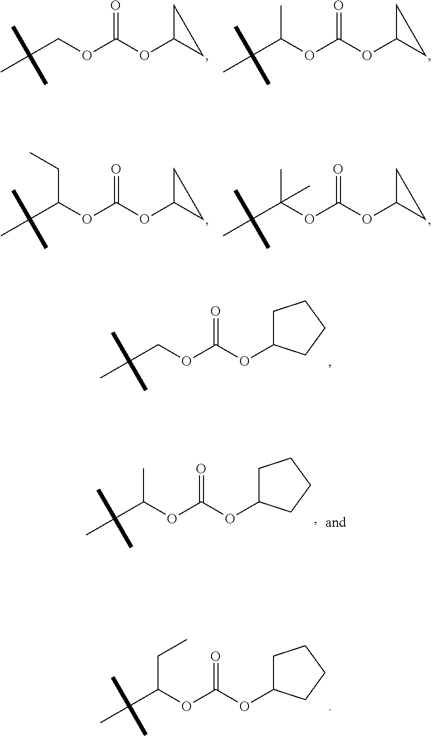

In other further embodiments, the compound is a prodrug of structure (I) having the following structure (III):

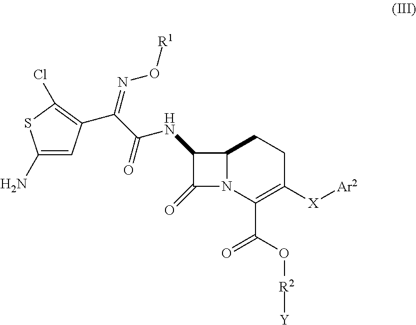

wherein R² and Y, taken together, are selected from:

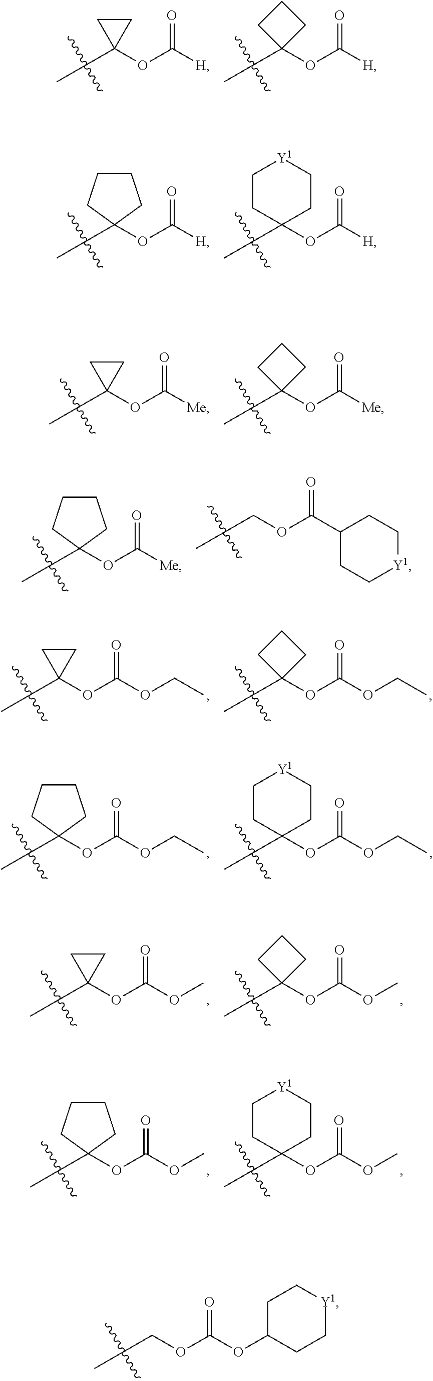

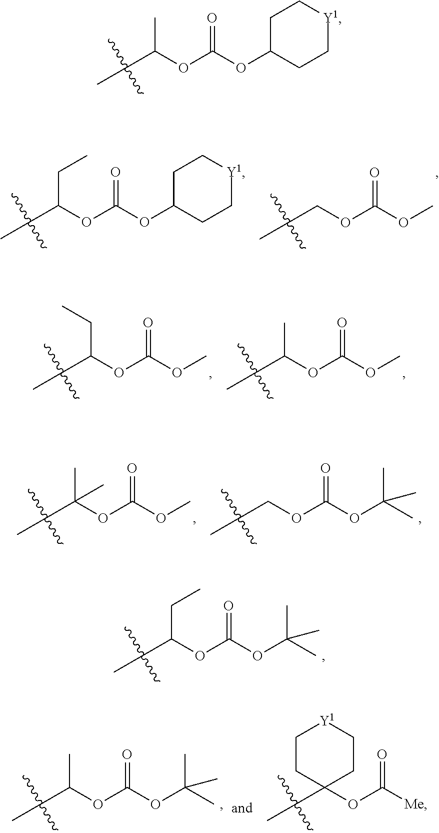

wherein Y¹ is —CH₂—, —O—, —S—, —SO₂—, —NH—, —NCH₃—, —NCH₂CH₃—, —NCH₂CH₂CH₃— or —NCH₂CF₃—.

In other further embodiments, X is —SCH₂— or —CH₂S—.

In other further embodiments, X is —SCH₂S—.

In other further embodiment, X is —SCH═CH— or —CH═CHS—.

In other further embodiments, X is —O—.

In other further embodiments, X is —C(═O)—.

In other further embodiments, X is —OCH₂— or —CH₂O—

In other further embodiments, X is alkylene. For example, X may be —CH₂— or —CH₂CH₂—.

In other further embodiments, X is substituted alkylene. For example, X may be selected from —CHF—, —CF₂—, —CHCH₃— and —C(CH₃)₂—.

In other further embodiments, X is alkenylene. For example, X may be —CH═CH—.

In other further embodiments, X is cycloalkyl. For example, X may be cyclopropyl.

In other further embodiments, Ar² is:

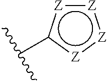

wherein:

each Z is independently selected from —CR⁵—, —S—, —O—, —N—, —NR⁶— such that the resulting ring is aromatic,

R⁵ is selected from hydrogen, chloro, bromo, fluoro, iodo, cyano, optionally substituted alkyl, optionally substituted alkenyl, optionally substituted alkynyl, optionally substituted cycloalkyl, optionally substituted aryl, optionally substituted heterocyclyl, optionally substituted heteroaryl, —SO₂R^{5a}, —SR^{5a}, —C(═O)R^{5a}, —C(═O)NR^{5a}R^{5b}, —NR^{5a}R^{5b} and —OR^{5a},
wherein:
R^{5a} is selected from hydrogen, optionally substituted alkyl, optionally substituted alkenyl, optionally substituted alkynyl, optionally substituted cycloalkyl, optionally substituted aryl, optionally substituted heterocyclyl and optionally substituted heteroaryl; and
R^{5b} is selected from hydrogen, optionally substituted alkyl, optionally substituted alkenyl, optionally substituted alkynyl, optionally substituted cycloalkyl, optionally substituted aryl, optionally substituted heterocyclyl and optionally substituted heteroaryl,
or R^{5a} and R^{5b}, together with the N atom to which they are attached, form an optionally substituted heterocyclyl or an optionally substituted heteroaryl; and

R⁶ is selected from hydrogen, optionally substituted alkyl, optionally substituted alkenyl, optionally substituted alkynyl, optionally substituted cycloalkyl, optionally substituted aryl, optionally substituted heterocyclyl and optionally substituted heteroaryl.

For example, in certain embodiments, Ar² is selected from:

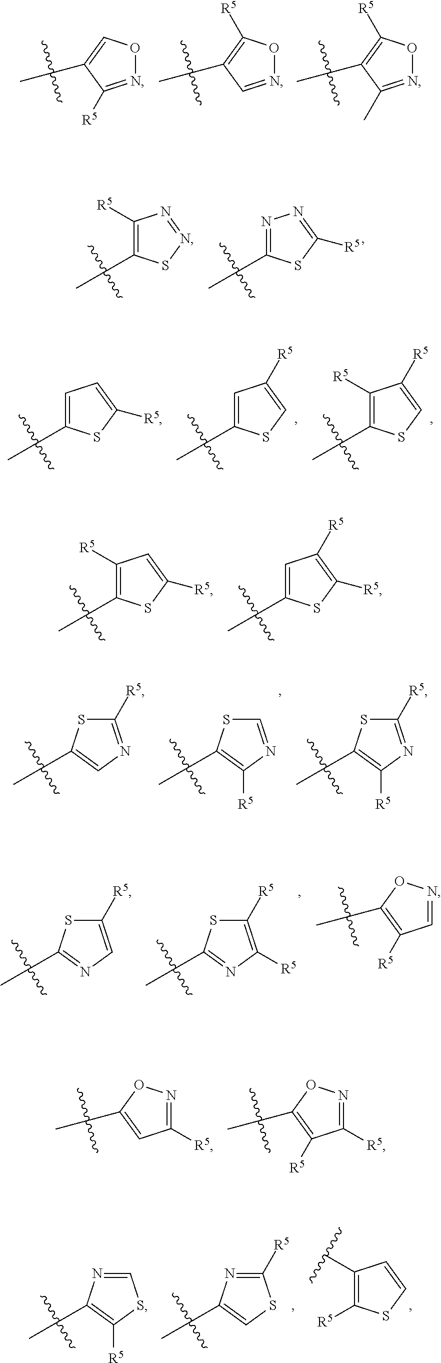

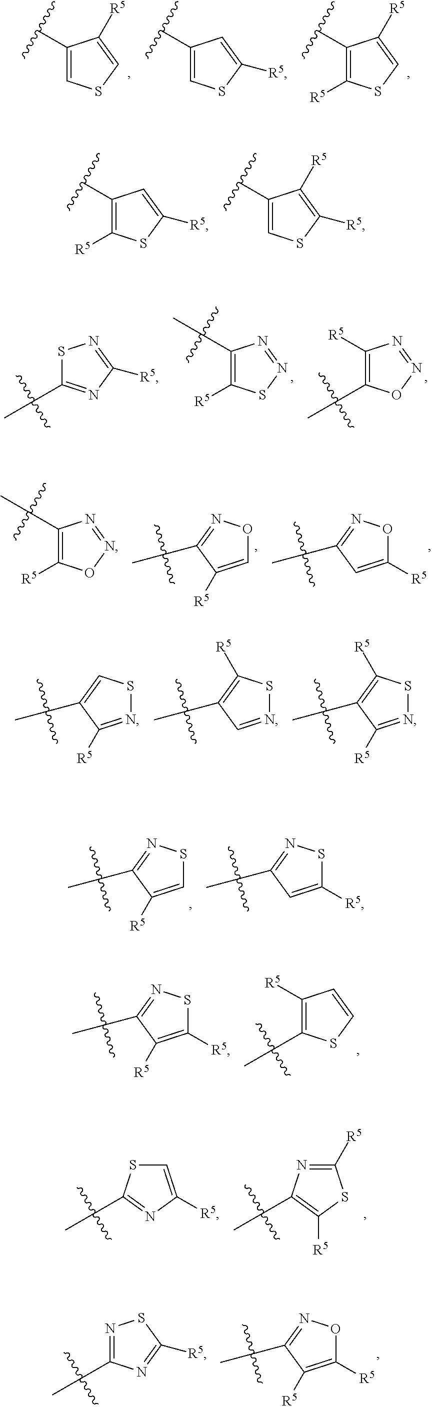

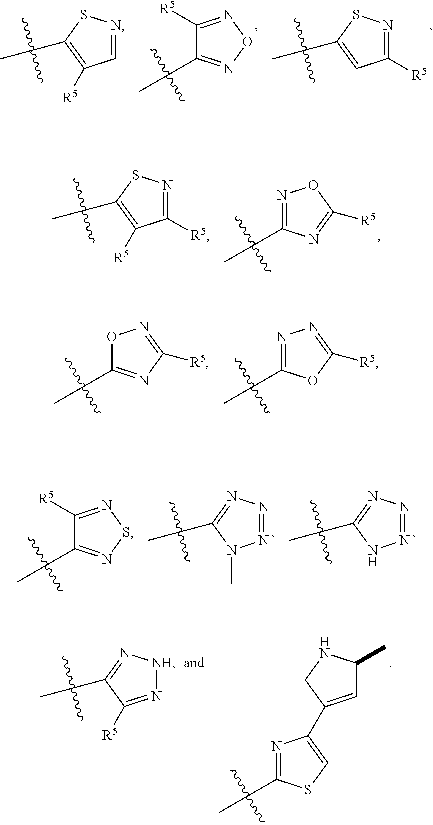

In certain of the foregoing certain embodiments, R⁵ is selected from hydrogen, chloro, fluoro, cyano, —CH₃, —CF₃, —SO₂CH₃, —SCH₃, —OCH₃, —OCF₃, —NH₂, —NHCH₃, —N(CH₃)₂, —NHCH₂CH₃, —N(CH₂CH₃)₂, —NHCH(CH₃)₂,

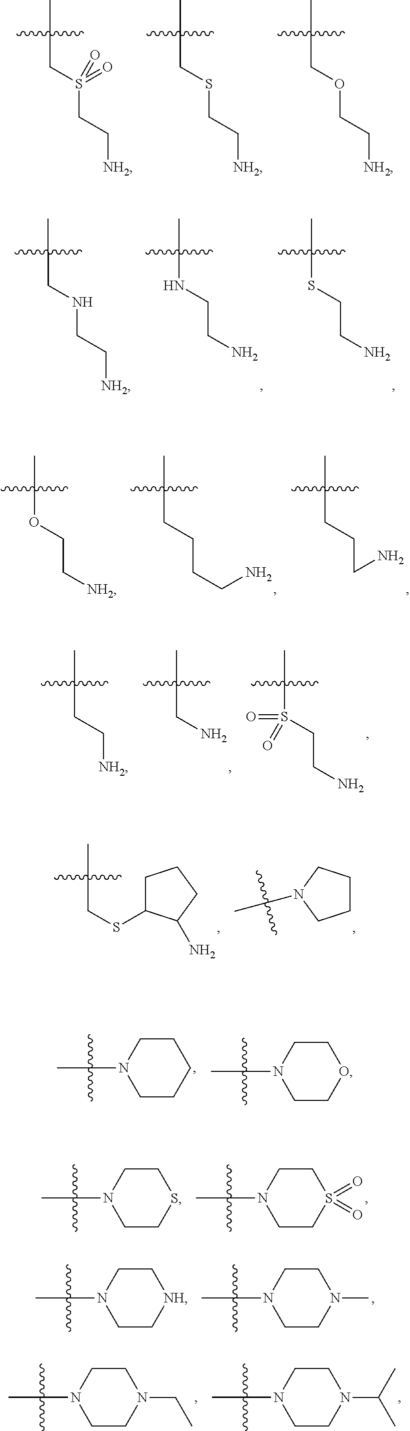

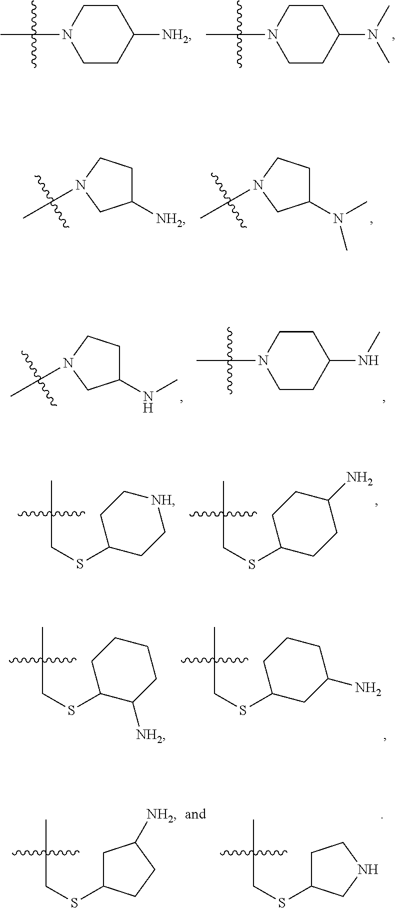

In other further embodiments, Ar² is:

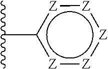

wherein:

each Z is independently selected from —CR⁵—, —S—, —O—, —N—, —NR⁶— such that the resulting ring is aromatic,

R⁵ is selected from hydrogen, chloro, bromo, fluoro, iodo, cyano, optionally substituted alkyl, optionally substituted alkenyl, optionally substituted alkynyl, optionally substituted cycloalkyl, optionally substituted aryl, optionally substituted heterocyclyl, optionally substituted heteroaryl, —SO₂R^{5a}, —SR^{5a}, —C(═O)R^{5a}, —C(═O)NR^{5a}R^{5b}, —NR^{5a}R^{5b} and —OR^{5a},

wherein:
R^{5a} is selected from hydrogen, optionally substituted alkyl, optionally substituted alkenyl, optionally substituted alkynyl, optionally substituted cycloalkyl, optionally substituted aryl, optionally substituted heterocyclyl and optionally substituted heteroaryl; and
R^{5b} is selected from hydrogen, optionally substituted alkyl, optionally substituted alkenyl, optionally substituted alkynyl, optionally substituted cycloalkyl, optionally substituted aryl, optionally substituted heterocyclyl and optionally substituted heteroaryl,
or R^{5a} and R^{5b}, together with the N atom to which they are attached, form an optionally substituted heterocyclyl or an optionally substituted heteroaryl; and

R⁶ is selected from hydrogen, optionally substituted alkyl, optionally substituted alkenyl, optionally substituted alkynyl, optionally substituted cycloalkyl, optionally substituted aryl, optionally substituted heterocyclyl and optionally substituted heteroaryl.

For example, in certain embodiments, Ar² is selected from:

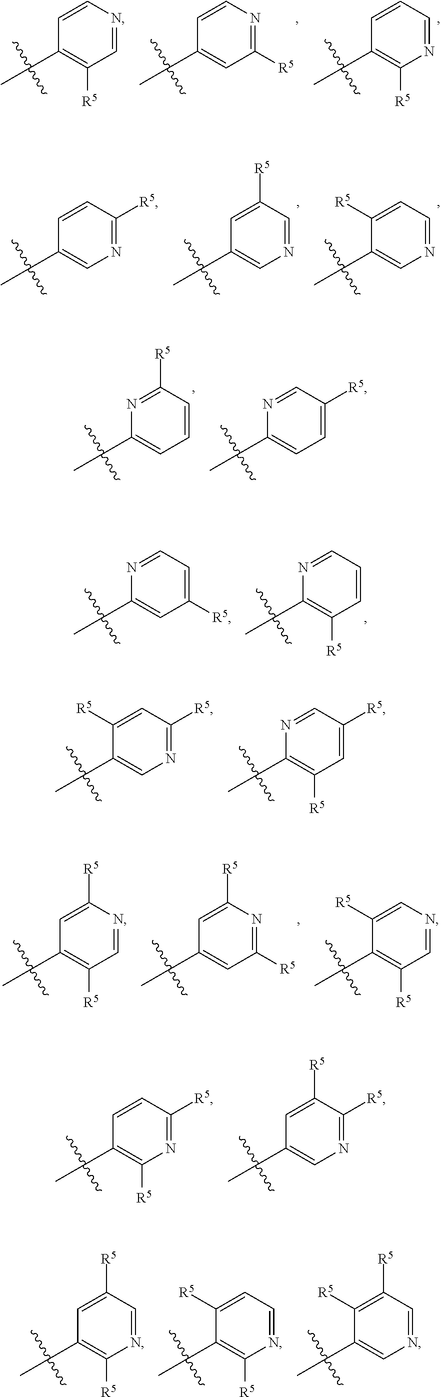

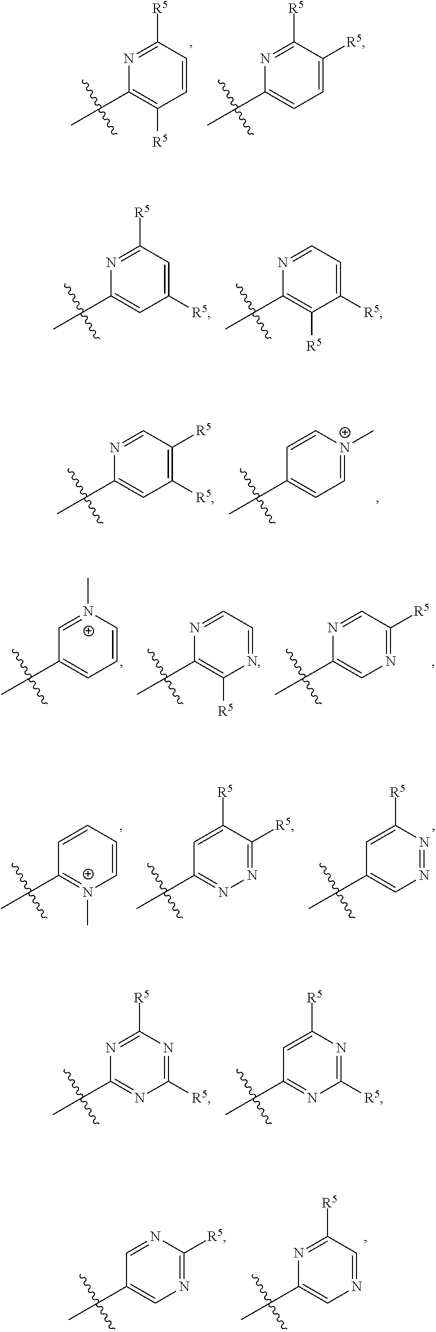

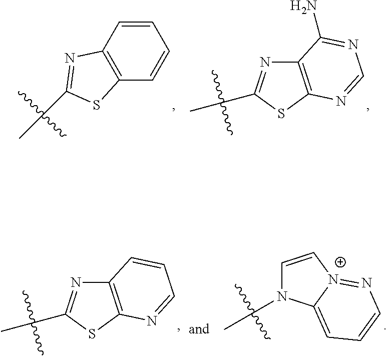

In certain of the foregoing certain embodiments, R⁵ is selected from hydrogen, chloro, fluoro, cyano, —CH₃, —CF₃, —SO₂CH₃, —SCH₃, —OCH₃, —OCF₃, —NH₂, —NHCH₃, —N(CH₃)₂, —NHCH₂CH₃, —N(CH₂CH₃)₂, —NHCH(CH₃)₂,

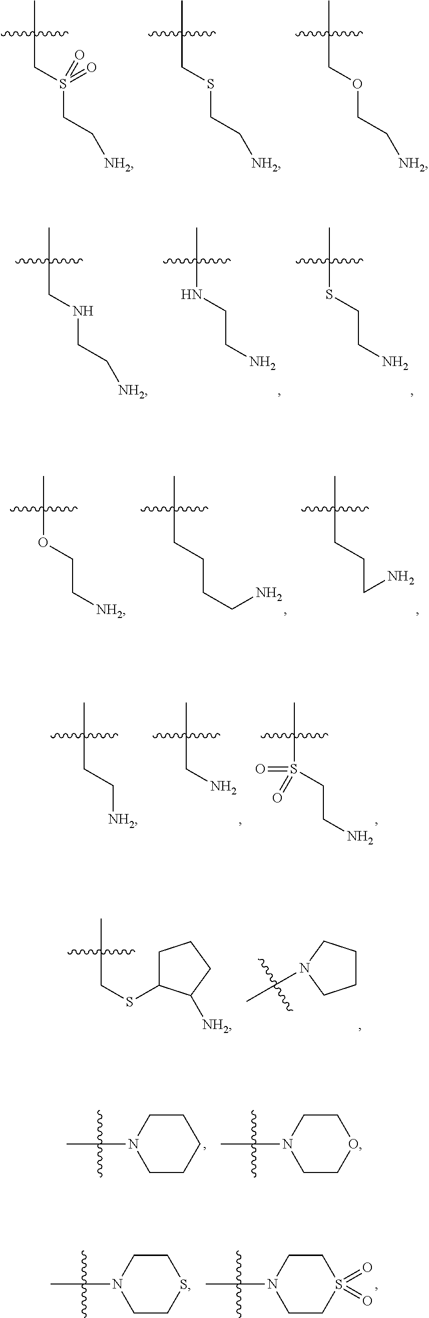

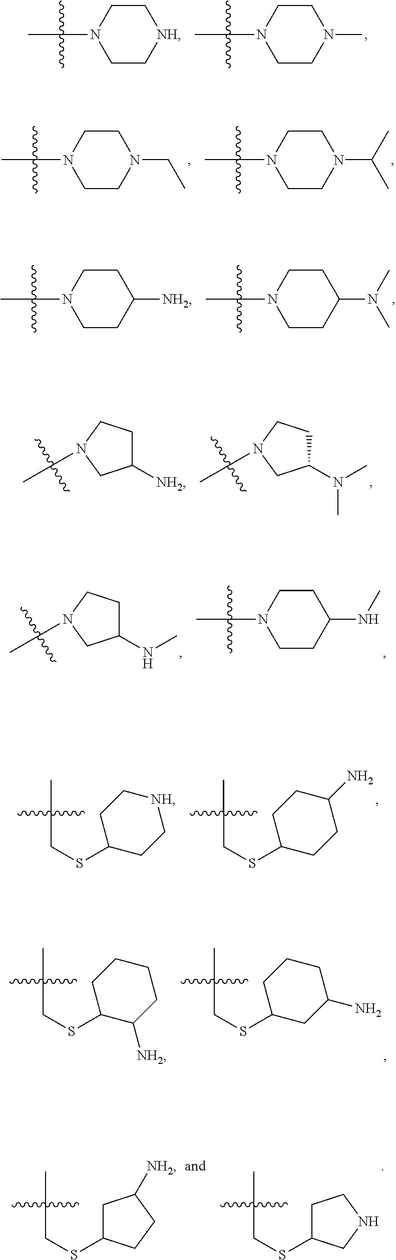

In another embodiment of the present invention, compounds having antibacterial activity are provided, the compounds having the following structure:

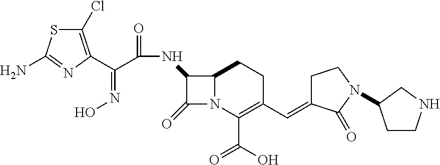

or a stereoisomer, pharmaceutically acceptable salt, ester, or prodrug thereof.

It is understood that any embodiment of the compounds of a structure disclosed herein, as set forth above, and any specific substituent set forth herein for an Ar², X, R¹ and R² group in the compounds of a structure disclosed herein, as set forth above, may be independently combined with other embodiments and/or substituents of compounds of a structure disclosed herein to form embodiments of the inventions not specifically set forth above. In addition, in the event that a list of substituents is listed for any particular substituent group in a particular embodiment and/or claim, it is understood that each individual substituent may be deleted from the particular embodiment and/or claim and that the remaining list of substituents will be considered to be within the scope of the invention.

For example, in one embodiment of compounds of structure (I), R¹ and R² are hydrogen, and the compounds have the following structure:

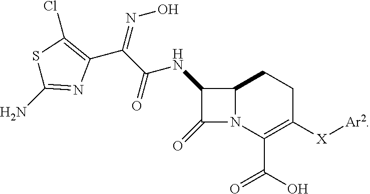

In another embodiment of compounds of structure (I), R¹ is alkyl (such as, for example, methyl) and R² is hydrogen, and the compounds have the following structure:

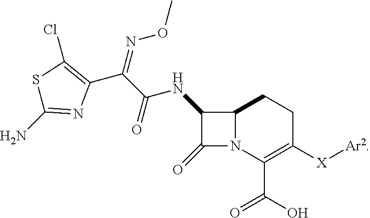

In another embodiment of compounds of structure (I), Ar² is a heteroaryl having 6 ring atoms selected from:

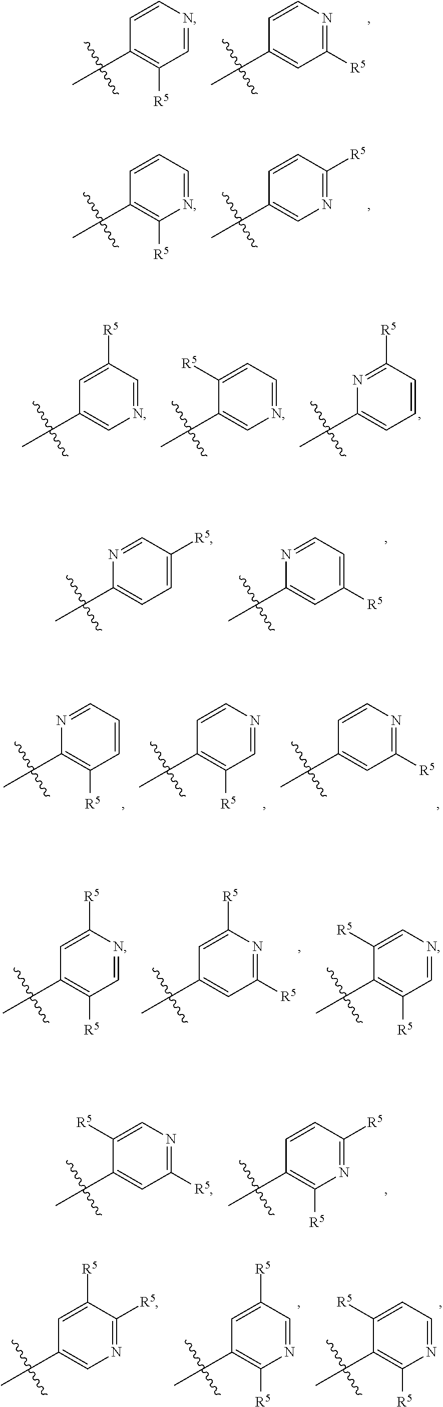

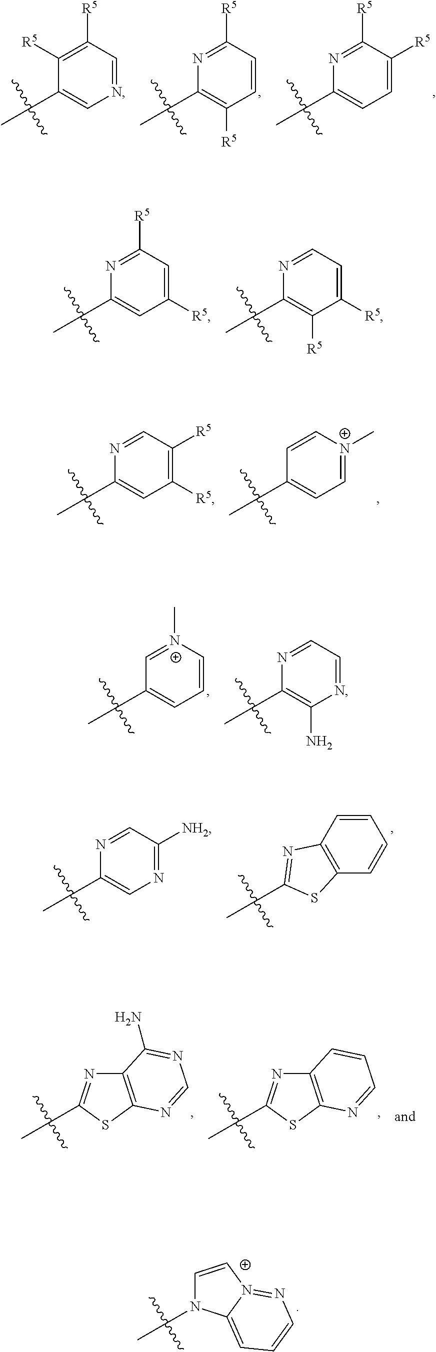

It is further understood that in the present description, combinations of substituents and/or variables of the depicted formulae are permissible only if such combinations result in stable compounds.

For the purposes of administration, the compounds of the present invention may be administered as a raw chemical or may be formulated as pharmaceutical compositions. Pharmaceutical compositions of the present invention comprise a compound of a structure disclosed herein and a pharmaceutically acceptable carrier, diluent or excipient. The compound of a structure disclosed herein is present in the composition in an amount which is effective to treat a particular disease or condition of interest—that is, in an amount sufficient to treat a bacterial infection, and preferably with acceptable toxicity to the patient. The antibacterial activity of compounds of a structure disclosed herein can be determined by one skilled in the art, for example, as described below. Appropriate concentrations and dosages can be readily determined by one skilled in the art.

The compounds of the present invention possess antibacterial activity against a wide spectrum of Gram-positive and Gram-negative bacteria, as well as enterobacteria and anaerobes. Representative susceptible organisms generally include those Gram-positive and Gram-negative, aerobic and anaerobic organisms whose growth can be inhibited by the compounds of the invention such as Staphylococcus, Lactobacillus, Streptococcus, Sarcina, Escherichia, Enterobacter, Klebsiella, Pseudomonas, Acinetobacter, Proteus, Campylobacter, Citrobacter, Nisseria, Baccillus, Bacteroides, Peptococcus, Clostridium, Salmonella, Shigella, Serratia, Haemophilus, Brucella and other organisms. In particular, the compounds of the present invention possess antibacterial activity against bacterial species resistant to conventional β-lactams, such as MRSA.

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition of the invention may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Compounds of the invention, or pharmaceutically acceptable derivatives thereof, may also be administered simultaneously with, prior to, or after administration of one or more other therapeutic agents. Such combination therapy includes administration of a single pharmaceutical dosage formulation which contains a compound of the invention and one or more additional active agents, as well as administration of the compound of the invention and each active agent in its own separate pharmaceutical dosage formulation. For example, a compound of the invention and the other active agent can be administered to the patient together in a single oral dosage composition such as a tablet or capsule, or each agent administered in separate oral dosage formulations. Where separate dosage formulations are used, the compounds of the invention and one or more additional active agents can be administered at essentially the same time, i.e., concurrently, or at separately staggered times, i.e., sequentially; combination therapy is understood to include all these regimens.

The following Examples illustrate various methods to make compounds of this invention, i.e., compounds having a structure disclosed herein, such as a compound of structure (I):

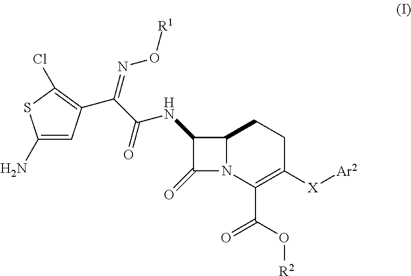

wherein Ar², X, R¹ and R² are described above. It is understood that one skilled in the art would be able to make these compounds by similar methods or by methods known to one skilled in the art. See, e.g., U.S. Pat. No. 5,077,287; Appelbaum, P. C., et al., Current Opinion in Microbiology, 2005, 8: 510-517; Bassetti, M., et al., Current Opinion in Investigational Drugs, 2003, 4(8): 944-952; Cooper, R. D. G, et al., Exp. Opin. Invest. Drugs, 1994, 3(8): 831-848; Elston, D. M., J. Am. Acad. Dermatol., 2007, 56(1): 1-16; Furuya, et al., Nature, 2006, 4: 36-45; Glinka, T. W., Current Opinion in Investigational Drugs, 2002, 3(2): 206-217; Glinka, T. W., et al., J. Antibiotics, 2000, 53(10): 1045-1052; Glinka, T. W., et al., Bioorganic & Medicinal Chemistry, 2003, 11: 591-600; Guzzo, P. R., et al., J. Org. Chem., 1994, 59(17): 4862-4867; Hecker, S. J., et al., J. Antibiotics, 2000, 53(11): 1272-1281; Hecker, S. J., et al., Antimicrobial Agents and Chemotherapy, 2003, 47(6): 2043-2046; Jackson, B. G., et al., Tetrahedron Letters, 1990, 31(44): 6317-6320; Jackson, B. G., Tetrahedron Letters, 2000, 56: 5667-5677; Lotz, B. T., et al., J. Org. Chem., 1993, 58(3): 618-625; Lowy, et al., J. Clinical Investigation, 2003, 111(9): 1265-1273; Misner, J. W., et al., Tetrahedron Letters, 2003, 44: 5991-5993; Mochida, K., et al., J. Antibiotics, 1989, 42(2): 283-292; and Rice, L. B., Am. J. Medicine, 2006, 119(6A): S11-S19. It is also understood that one skilled in the art would be able to make in a similar manner as described below other compounds of a structure disclosed herein not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Advanced Organic Chemistry: Reactions, Mechanisms, and Structure, 5th edition (Wiley, December 2000)) or prepared as described herein.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

Furthermore, all compounds of the invention which exist in free base or acid form can be converted to their pharmaceutically acceptable salts by treatment with the appropriate inorganic or organic base or acid by methods known to one skilled in the art. Salts of the compounds of the invention can be converted to their free base or acid form by standard techniques.

The following Examples are provided for purposes of illustration, not limitation.

### EXAMPLES

### Example 1

### Generation of the Chiral Carbacephem Core

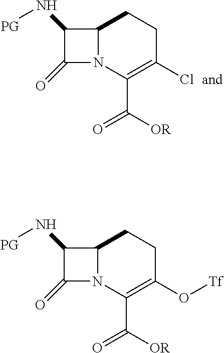

### (6R,7S)-7-protected amino-3-chloro (or triflate)-8-oxo-1-azabicyclo[4.2.0]oct-2-ene-2-carboxylate esters

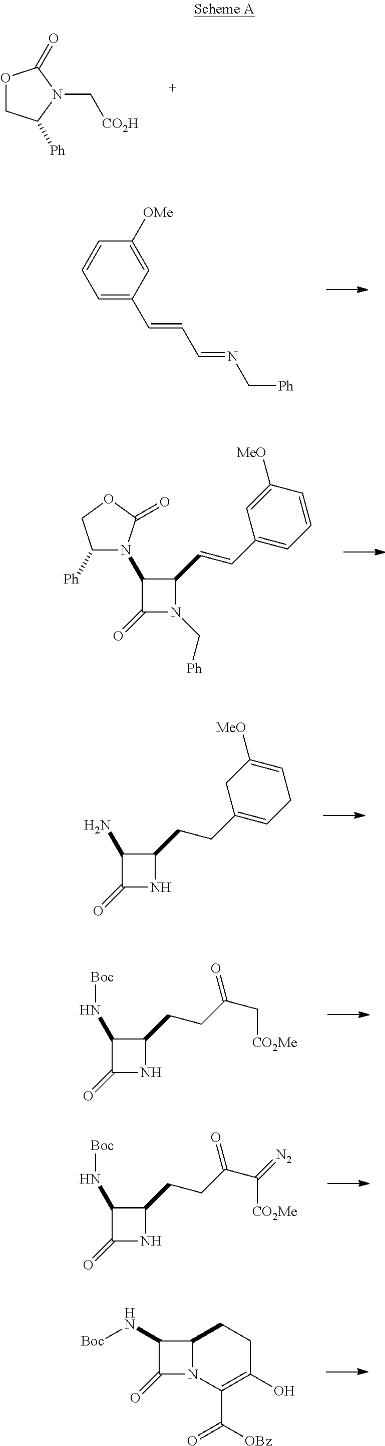

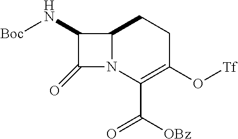

The final compound in the Evans scheme can be converted to several important carbacephem intermediates with selective protecting group manipulations. The 3-pos triflate can be displaced by nucleopliles to give sulfur linked groups, see, e.g., Ternansky, R. J., et al., J. Med. Chem., 1993, 36: 1971-1976 and Hatanaka, M. et al., Tetrahedron Letters, 1983, 24(44): 4837-4838. The triflate can also be converted to an alkene by Stille reaction to give double bond linked groups at the 3-position. The Boc can be removed for coupling to an acid at the 7-position. See, e.g., Evans, D. A., et al., Tetrahedron Letters, 1985: 3783-3787 and Evans, D. A., et al., Tetrahedron Letters, 1985: 3787-3790.

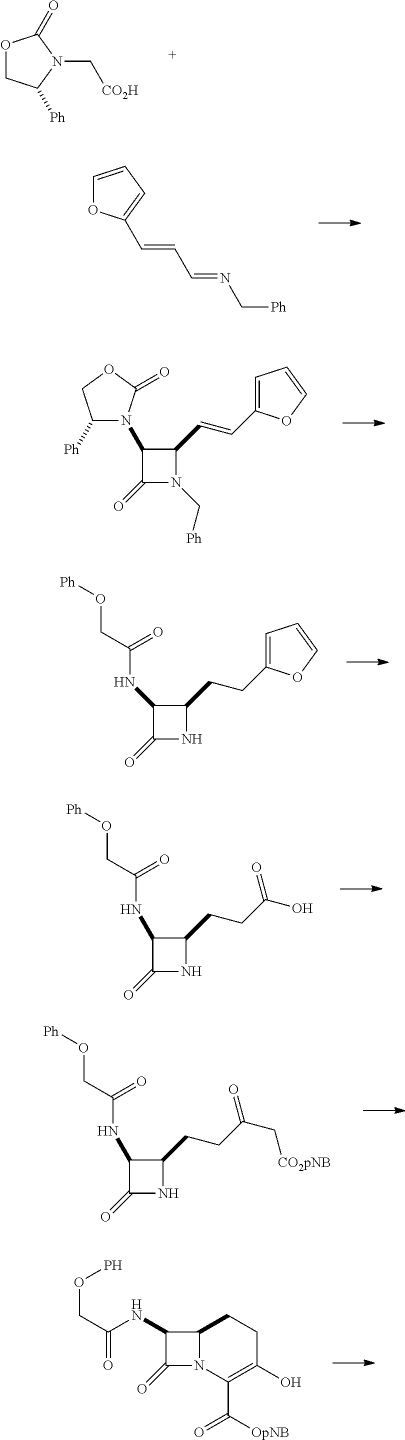

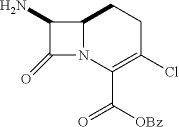

The Bodurow method gives the needed intermediate for the schemes below for 3-position S-linked analogues. The Bz protected ester can be converted to the free acid by hydrogenation or saponification. See, e.g., Bodurow, C. C, et al., Tetrahedron Letters, 1989: 2321-2324.

### Example 2

### Stille Coupling from the 7-Position Triflate and Transformation to the Bromomethyl Ketone

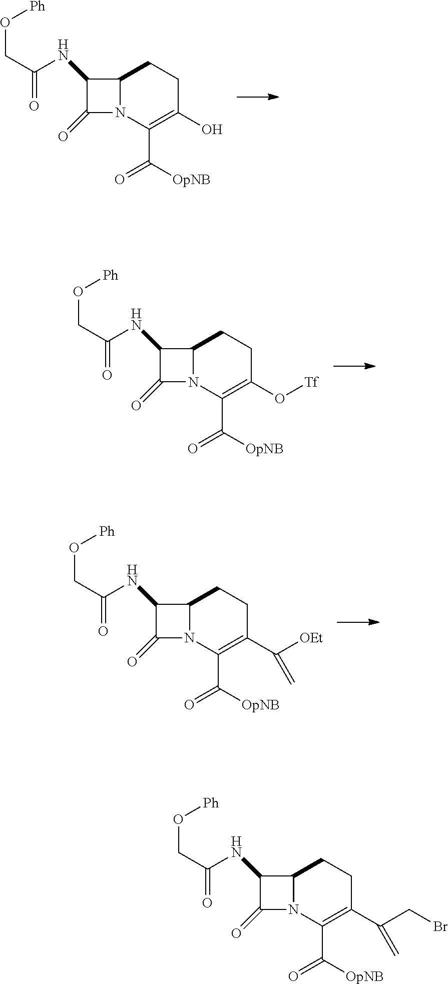

### See, e.g., Hornback, W. J., American Chemical Society National Meeting, Abstract 153, Washington, 1990.

### Example 3

### Carbonylation from the 7-Position Triflate

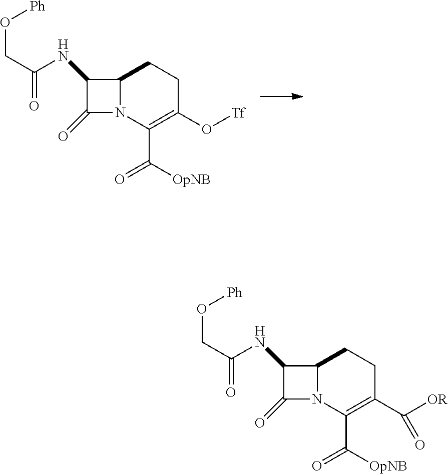

G. K. Cook, W. J. Hornback, C. L, Jordan, J. H, McDonald III and J. E. Munroe, J. Org. Chem., 1989, 54: 5828-5830.

### Example 4

### Conversion of 7-Position Triflate to Carboxylic Acid, Vinyl, Aldehyde and Methylene-Linked Heterocycles

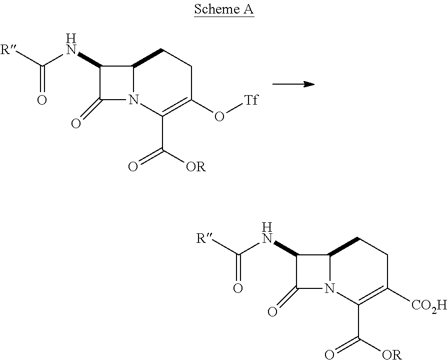

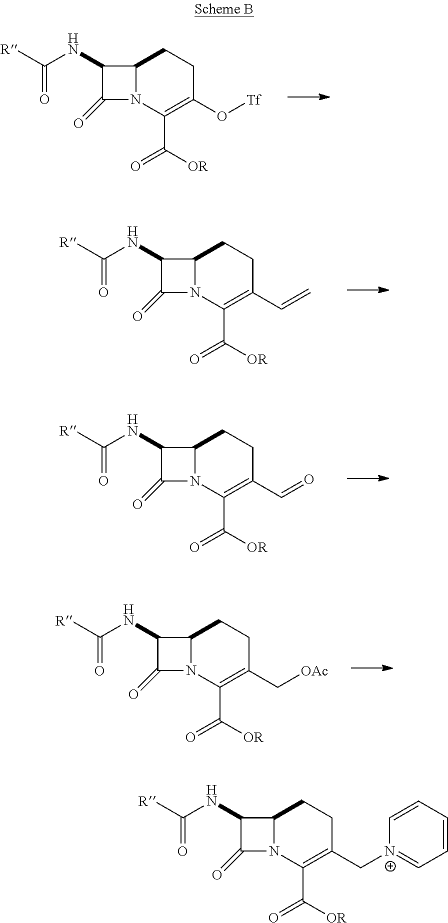

See, e.g., Blaszczak, L. C., et al., J. Med. Chem., 1990, 33(6): 1656-62.

### Example 5

### Coupling at 7-Position

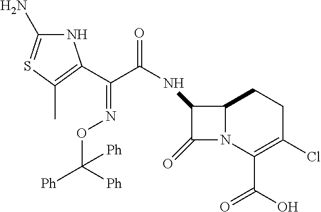

### (7R)-7-[(Z)-2-(2-amino-5-methylthiazol-4-yl)-2-(triphenylmethoxyimino]acetamido]-3-chloro-8-oxo-1aza-bicyclo[4.2.0]oct-2-ene-2-earboxylate triethylamine salt

To a solution of bis-(2-benzothiazolyl)-disulfide (0.013 mol) in dichloromethane (100 mL) was added triphenylphosphine (0.013 mol). The mixture was stirred for 15 min after which (Z)-2-(2-amino-5-methylthiazol-4-yl)-2-(triphenylmethoxyimino)acetic acid (0.010 mol) was added. The mixture was stirred for 1 h and was cooled to 0° C. In a separate flask, (7R)-7-amino-3-chloro-8-oxo-1aza-bicyclo[4.2.0]oct-2-ene-2-carboxylic acid trifluoroacetic acid salt (0.008 mol) was suspended in dichloromethane (50 mL) and triethylamine (4.0 g, 0.04 mol) was added. The suspension was stirred for 0.5 h at rt and then was transferred to the flask containing the activated ester of 7-[(Z)-2-(2-amino-thiazolyl-4)-2-trityloxyimino]carboxylic acid. The resulting clear solution was allowed to warm to rt and was stirred for 48 h. The reaction mixture was washed twice with 100 mL portions of water, and the organic layer was separated, dried over anhydrous MgSO₄, filtered and concentrated to approximately 50 mL. The oily residue was treated with diethyl ether (250 mL), and the solid was filtered and dried giving crude product. HPLC analysis indicated that it contained approximately 0.004 mol of the desired compound as the triethylamine salt.

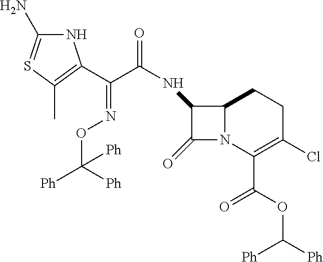

### (7R)-7-[(Z)-2-(2-amino-5-methylthiazol-4-yl)-2-(triphenylmethoxyimino]acetamido]-3-chloro-8-oxo-1aza-bicyclo[4.2.0]oct-2-ene-2-carboxylate diphenylmethyl ester

The crude (7R)-7-[(Z)-2-(2-amino-5-methylthiazol-4-yl)-2-triphenyl methoxyimino]-acetamido]-3-chloro-oxo-1-aza-bicyclo[4.2.0]oct-2-ene-2-carboxylic acid triethylamine salt (0.004 mol) was dissolved in dichloromethane (200 mL) and was washed twice with 50% NaPO₄:H₂O and then with water. The organic layer was dried over anhydrous MgSO₄, filtered and treated with diphenyldiazomethane solution in dichloromethane (40 mL of 0.5 mol/L solution, 0.02 mol), followed by stirring at rt for 1 h. The reaction mixture was concentrated to dryness and the residue was dissolved in ethyl acetate (20 mL). The ethyl acetate solution was then chromatographed on silica gel (200 g). Nonpolar byproducts were eluted with ethyl acetate:hexane (1:6), and the product with ethyl acetate:hexane (1:1). After evaporation, the title ester was obtained. HPLC indicated that it contained approximately 0.004 mol of the desired product.

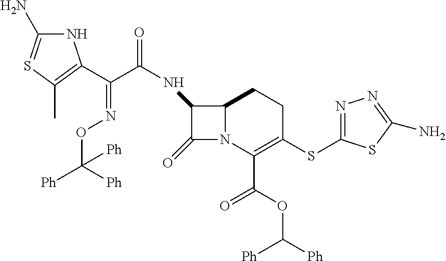

### (7R)-7-[(Z)-2-(2-amino-5-methylthiazol-4-yl)-2-(triphenylmethoxyimino]acetamido]-3-[5-amino-1,3,4-thiadiazol-2-ylthio]-8-oxo-1-aza-bicyclo[4.2.0]oct-2-ene-2-carboxylate diphenylmethyl ester

To a solution of 5-amino-1,3,4-thiadiazole-2-thiol (0.0045 mol) in dimethylformamide (25 mL) was added potassium carbonate (1.0 g, 0.0076 mol). The mixture was stirred for 1 h at rt after which (7R)-7[(Z)-2-(2-amino-5-methylthiazol-4-yl)-2-(triphenylmethoxyimino]acetamido]-3-chloro-8-oxo-1aza-bicyclo[4:2.0]oct-2-ene-2-carboxylate diphenylmethyl ester (0.0039 mol) was added. Stirring was continued for 18 h. The mixture was partitioned between ethyl acetate (50 mL) and water (50 mL). The organic layer was separated, washed with water (30 mL), dried over anhydrous MgSO₄ and the solvent was removed with a rotary evaporator. The resultant thick oil was treated with diethyl ether (50 mL) and the solid which formed was filtered and dried to give ca. 0.0028 mol of crude product.

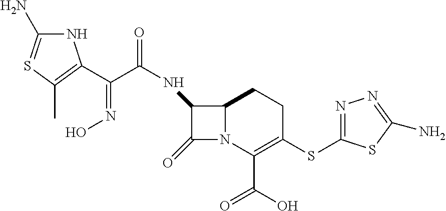

### (7R)-7-[(Z) 2-(2-amino-5-methylthiazol-4-yl)-2-(hydroxyimino]acetamido]-3-[5-amino-1,3,4-thiadiazol-2-ylthio]-8-oxo-1-aza-bicyclo[4.2.0]oct-2-ene-2-carboxylic acid

A solution of trifluoroacetic acid (10 mL), triethylsilane (5 mL) and dichloromethane (10 mL) was cooled to 0° C. and (7R)-7-[(Z)-2-(2-amino-5-methylthiazol-4-yl)-2-(triphenylmethoxyimino]acetamido]-3-[5-amino-1,3,4-thiadiazol-2-ylthio]-8-oxo-1-aza-bicyclo[4.2.0]oct-2-ene-2-carboxylate diphenylmethyl ester from the previous step (ca. 0.0028 mol) was added in portions. The reaction mixture was stirred for 3 h at 0° C., allowed to warm up to rt and evaporated to dryness. The residue was treated with diethyl ether (50 mL) and the solid that formed was filtered and dried to give crude product. The crude product was purified with Diaion HP-20 resin initially with water elution until the pH was neutral, after which the product was eluted with acetonitrile:water 80:20. The solvent was evaporated to give the ca. 0.0013 mol of the title compound.

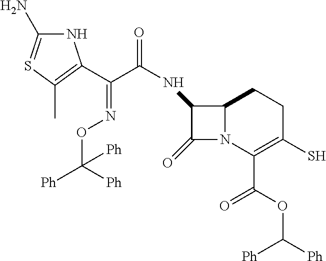

### (7R)-7-[(Z)-2-(2-amino-5-methylthiazol-4-yl)-2-(triphenylmethoxyimino]acetamido]-3-mercapto-8-oxo-1-aza-bicyclo[4.2.0]oct-2-ene-2-carboxylate diphenylmethyl ester

A solution of (7R)-7-[(Z)-2-(2-amino-5-methylthiazol-4-yl)-2-(triphenyl methoxyimino]-acetamido]-3-chloro-8-oxo-1-aza-bicyclo[4.2.0]oct-2-ene-2-carboxylate diphenylmethyl ester (0.0036 mol) in dimethylformamide (40 mL) was cooled to −20° C. and a solution of ammonium sulfide in water (20%, 5.7 mL) was added drop-wise. The mixture was stirred at −20° C. for 4 h and then was poured into pH 3 phosphate buffer (100 mL). The resulting solid was filtered, washed with water and dried to afford the crude title compound (ca. 0.0036 mol).

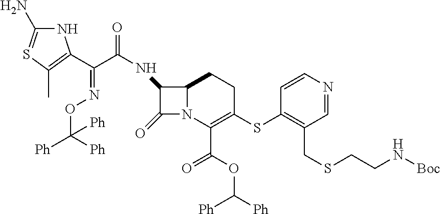

### (7R)-7-((Z)-2-(2-amino-5-methylthiazol-4-yl)-2-(triphenylmethoxyimino]acetamido]-3-(3-(N-tert-butoxycarbonylaminoethylthiomethyl)pyrid-4-ylthio]-8-oxo-1-aza-bicyclo[4.2.0]oct-2-ene-2-carboxylate diphenylmethyl ester

To a solution of (7R)-7-[(Z)-2-(2-amino-5-methylthiazol-4-yl)-2-(triphenyl methoxyimino]-acetamido]-3-mercapto-8-oxo-1-aza-bicyclo[4.2.0]oct-2-ene-2-carboxylate diphenylmethyl ester (0.0036 mol) in dimethylformamide (30 mL) was added 3-(N-tert-butoxycarbonylaminoethylthiomethyl)-4-chloropyridine (1.3 g, 0.0043 mol) at rt. After stirring overnight, the reaction mixture was treated with water (200 mL), and the solid that formed was filtered and dried to afford the crude title compound (ca. 0.0027 mol).

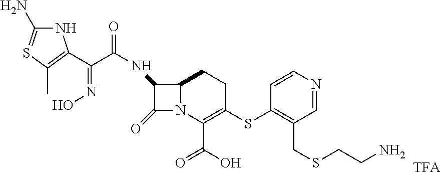

### (7R)-7-[(Z)-2-(2-amino-5-methylthiazol-4-yl)-2-(hydroxyimino]acetamido]-3-[3-(aminoethylthiomethyl)pyrid-4-ylthio]-8-oxo-1-aza-bicyclo[4.2.0]oct-2-ene-2-carboxylate, trifluoroacetic acid salt

A solution of trifluoroacetic acid (10 mL), triethylsilane. (5 mL) and dichloromethane (10 mL) was cooled to 0° C. and (7R)-7-[(Z)-2-(2-amino-5-methylthiazol-4-yl)-2-(triphenylmethoxyimino]acetamido]-3-[3-(N-tertbutoxycarbonylaminoethylthiomethyl)-pyrid-4-ylthio]-8-oxo-1aza-bicyclo[4.2.0]oct-2-ene-2-carboxylate diphenyl methyl ester (ca. 0.0027 mol) was added in portions. The reaction mixture was stirred at 0° C. for 6 h, was allowed to warm to rt and was evaporated to dryness. The residue was treated with diethyl ether (50 mL), and the solid that formed was filtered and dried to give crude product. The crude product was purified with Diaion HP-20 resin with water elution until the pH was neutral, and thereafter with acetonitrile:water 80:20, to give the product (ca. 0.0002 mol).

### Example 6

### Ceftobiprole Style Methylene Linker at 7-Position

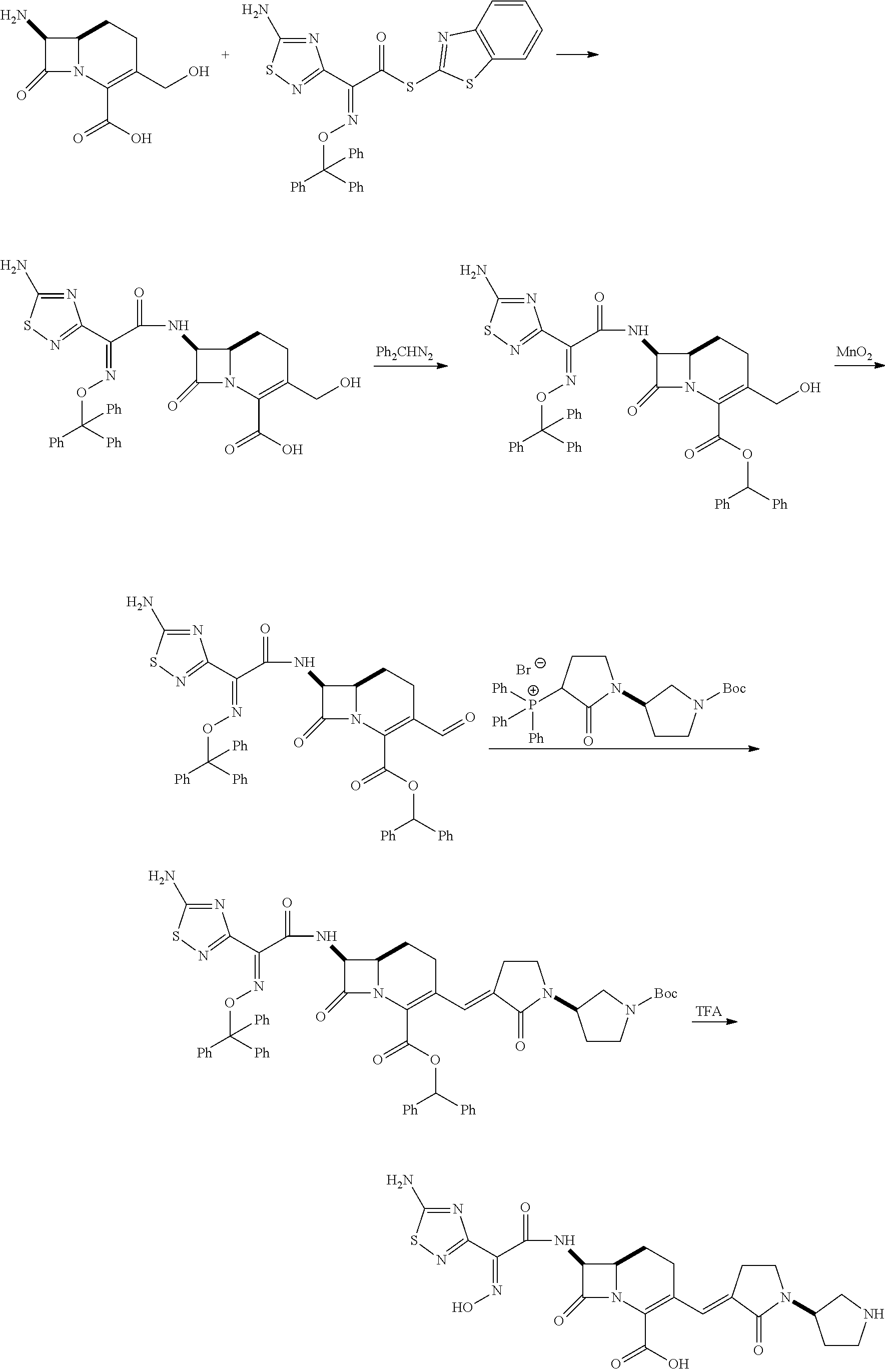

The procedures used to produce ceftobiprole can be applied to carbacephem compounds. See, e.g., Canadian Patent No. 2408941, European Patent Nos. 1 289 998 and 1 435 357, Japanese Patent No. 2003535059, U.S. Patent Application No. 2002/019381, U.S. Pat. No. 6,504,025 and International PCT Application Publication No. WO 01/90111.

### Example 7

### Preparation of Alkylene Linked Groups at 7-Position

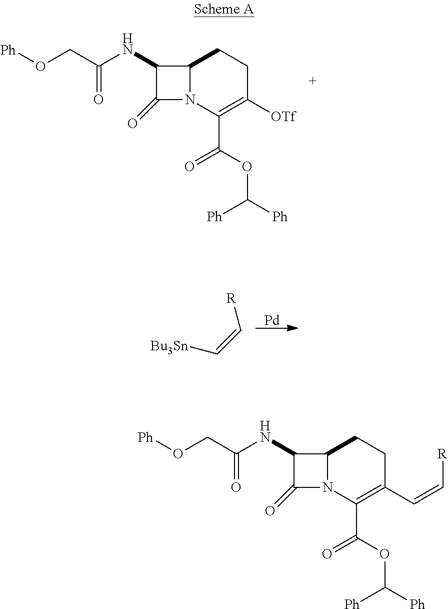

See, e.g., U.S. Pat. No. 4,855,418 and Farina, V., et al, Tetrahedron Letters, 1988, 29(47): 6043-6.

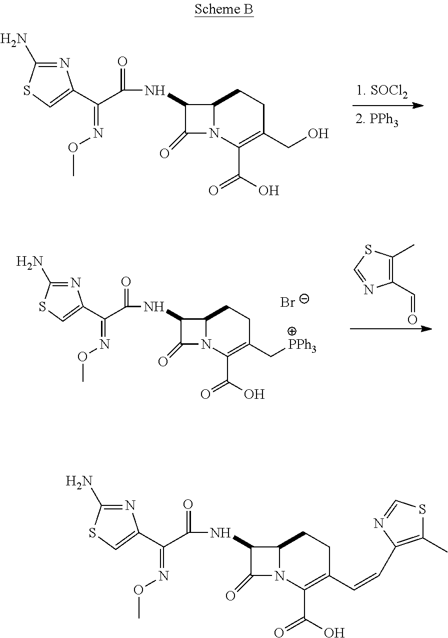

See, e.g., Chinese Patent No. 1763046; Xiao, T. Z., et al., Chinese J. Pharmaceuticals, 2004, 35(7): 388-390; Kim, G. T., et al., J. Antibiotics, 2004, 57(7): 468-472; International PCT Application Publication No. WO 2005/100330; International PCT Application Publication No. WO 2005/100367; and Hanaki, H., et al., J. Antibiotics, 2005, 58(1): 69-73.

### Example 8

### Methods A, B, C, D and E

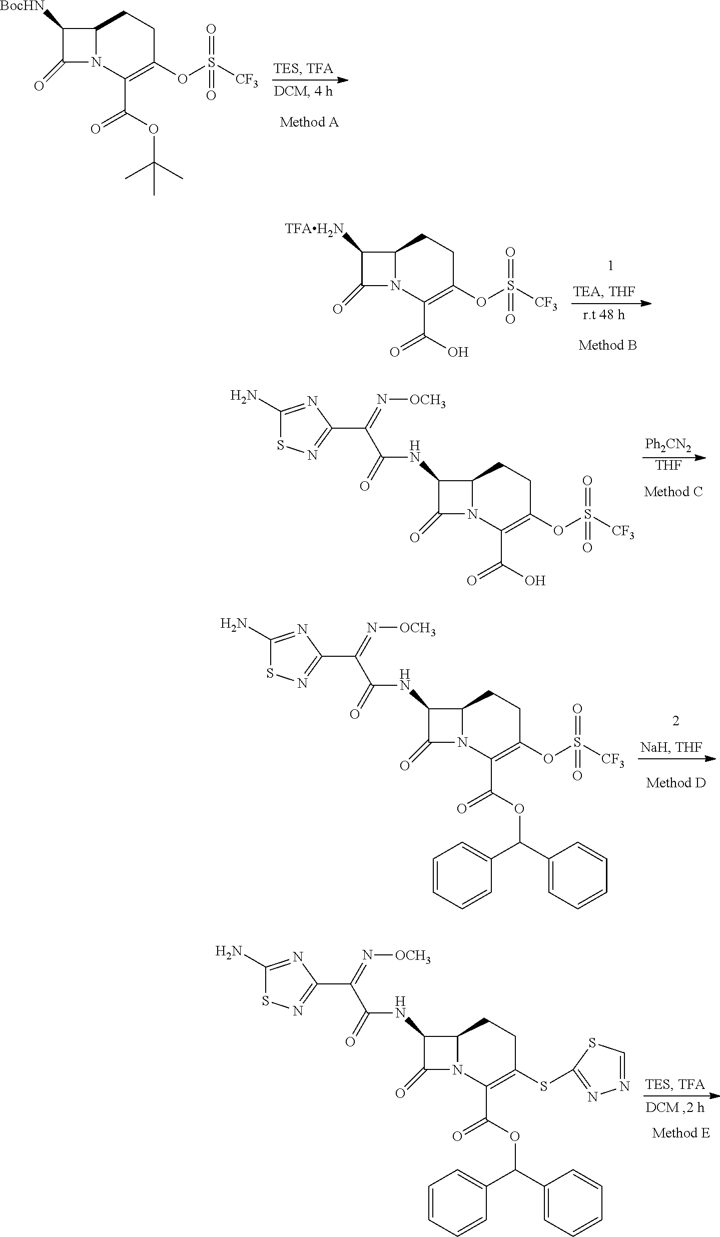

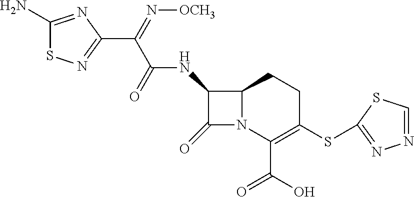

Method A: To a solution of (6R,7S)-tert-butyl-7-(tert-butoxycarbonylamino)-8-oxo-3-(trifluoromethylsulfonyloxy)-1-aza-bicyclo[4.2.0]oct-2-ene-2-carboxylate (see Example 9) (502 mg, 1.04 mmol) in dichloromethane (DCM) (10 mL) was added triethylsilane (TES) (116 mg, 10 mmol). The mixture was cooled down to 0° C. and 2,2,2-trifluoroacetic acid (TFA) (10 mL) was added. Then, the mixture was allowed to warm to rt and stirred for 4 h, concentrated under reduced pressure and washed with petroleum ether to obtain white solid (580 mg crude solid). The resulting product was used without further purification.

Method B: (6R,7S)-7-amino-8-oxo-3-(trifluoromethylsulfonyloxy)-1-aza-bicyclo[4.2.0]oct-2-ene-2-carboxylic acid (1.5 g, 4.3 mmol) was suspended in tetrahydrofuran (THF) (15 mL) and triethylamine (2.1 g, 21 mmol) was added. The suspension was stirred for 0.5 h at rt, and (Z)-S-benzo[d]thiazol-2-yl-2-(5-amino-1,2,4-thiadiazol-3-yl)-2-(methoxyimino)ethanethioate 1 (see Example 10) (1.4 g, 4.3 mmol) was added with stirring at 0° C. The mixture was allowed to warm to rt and stirred for 48

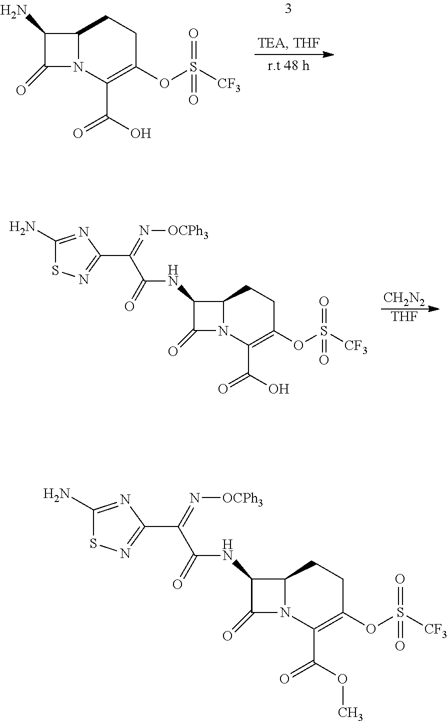

Similar to Method B, (6R,7S)-7-amino-8-oxo-3-(trifluoromethylsulfonyloxy)-1-aza-bicyclo[4.2.0]oct-2-ene-2-carboxylic acid (1.5 g, 4.3 mmol) was suspended in tetrahydrofuran (THF) (15 mL) and triethylamine (2.1 g, 21 mmol) was added. The suspension was stirred for 0.5 h at rt, and (Z)-S-benzo[d]thiazol-2-yl-2-(5-amino-1,2,4-thiadiazol-3-yl)-2-(trityloxyimino)ethanethioate 3 (see Example 10) (2.5 g, 4.3 mmol) was added with stirring at 0° C. The mixture was allowed to warm to rt and stirred for 48 h and concentrated under reduced pressure. The residue was dissolved in ethyl acetate (EA), washed with dilute aqueous HCl (pH=4), washed with brine, dried with NaSO₄, filtered and then the solvent was removed in vacuo to give 3.6 g as a slight yellow solid. The resulting product was used without further purification.

To a solution of (6R,7S,Z)-7-(2-(5-amino-1,2,4-thiadiazol-3-yl)-2-(trityloxyimino)acetamido)-8-oxo-3-(trifluoromethylsulfonyloxy)-1-aza-bicyclo[4.2.0]oct-2-ene-2-carboxylic acid (55 mg, 0.074 mmol, 1.0 eq) in THF (1 mL) at 0° C., a solution of CH₂N₂ in Et₂O (10 mL, 0.74 mol, 10 eq) was added. The resulted suspension was stirred for 6 h. The reaction mixture was concentrated in vacuo to get the crude product. The crude product was purified by column chromatography on silica gel using PE:EA=4:1 as an eluent to furnish the desired product as a light yellow solid in 18% yield.

### Method G

h and concentrated under reduced pressure. The residue was dissolved in ethyl acetate (EA), washed with dilute HCl aqueous (pH=4), washed with brine, dried with NaSO₄, filtered and then the solvent was removed in vacuo to give 2.6 g of slight yellow solid. The resulting product was used without further purification.

Method C: (6R,7S,Z)-7-(2-(5-amino-1,2,4-thiadiazol-3-yl)-2-(methoxyimino)acetamido)-8-oxo-3-(trifluoromethylsulfonyloxy)-1-aza-bicyclo[4.2.0]oct-2-ene-2-carboxylic acid (2.6 g crude solid) was dissolved in THF and (diazomethylene)dibenzene (4.9 g, 25 mmol) was added dropwise. The reaction mixture was stirred for 2 h at rt. Then, the solvent was concentrated in vacuo and petroleum ether (PE) was added to obtain precipitation. The resulting product was used without further purification.

Method D: A solution of 1,3,4-thiadiazole-2-thiol 2 (47 mg 0.39 mmol) in dry THF was cooled down in ice and treated with NaH (14 mg, 0.36 mmol). After 10 min, the suspension was added by syringe to the solution of (6R,7S,Z)-benzhydryl-7-(2-(5-amino-1,2,4-thiadiazol-3-yl)-2-(methoxyimino)acetamido)-8-oxo-3-(trifluoromethylsulfonyloxy)-1-aza-bicyclo[4.2.0]oct-2-ene-2-carboxylate (226 mg, 0.33 mmol) in dry tetrahydrofuran (THF) at −30° C. The temperature was allowed to reach 0° C. over 2 h. The mixture was washed with cold HCl (aq), brine, dried with Na₂SO₄, filtered and concentrated. The residue was purified by gel column chromatography (EA:PE=2:1) to give white solid (114 mg, 53%).

Method E: A solution of triethylsilane (TES) (0.5 mL), 2,2,2-trifluoroacetic acid (TFA) (1 mL) and dichloromethane (DCM) (1 mL) was cooled down to 0° C. and (6R,7S,Z)-benzhydryl-3-(1,3,4-thiadiazol-2-ylthio)-7-(2-(5-amino-1,2,4-thiadiazol-3-yl)-2-(methoxyimino)acetamido)-8-oxo-1-aza-bicyclo[4.2.0]oct-2-ene-2-carboxylate (114 mg, 0.17 mmol) was added in portions. The reaction mixture was stirred for 3 h at 0° C. and then evaporated to dryness, washed with diethyl ether to obtain (6R,7S,Z)-3-(1,3,4-thiadiazol-2-ylthio)-7-(2-(5-amino-1,2,4-thiadiazol-3-yl)-2-(methoxyimino)acetamido)-8-oxo-1aza-bicyclo[4.2.0]oct-2-ene-2-carboxylic acid (82 mg crude solid), purified by Prep-HPLC to give 26 mg white solid, yield: 31%.

### Method F

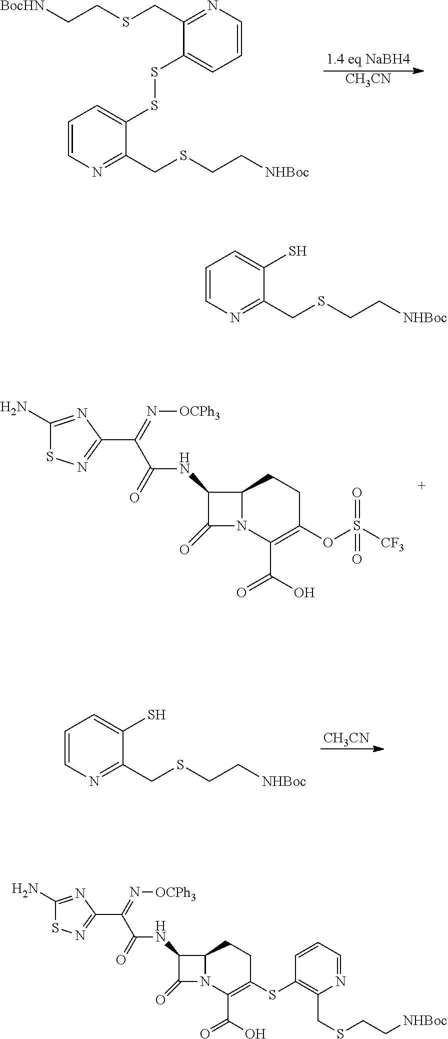

To a solution of di-tert-butyl-2,2′-(3,3′-disulfanediylbis(pyridine-3,2-diyl)bis(methylene)bis(sulfanediyl)bis(ethane-2,1-diyl)dicarbamate (156 mg) in acetonitrile (10 mL) was added NaBH₄ (14.4 mg). The mixture was stirred at rt for 18 h and the mixture was used for next step without purification. (6R,7S,Z)-7-(2-(5-amino-1,2,4-thiadiazol-3-yl)-2-(trityloxyimino)acetamido)-8-oxo-3-(trifluoromethylsulfonyloxy)-1-aza-bicyclo[4.2.0]oct-2-ene-2-carboxylic acid (see Method F) was added to the solution and stirred for 3.5 h and evaporated to dryness. The residue was taken to column chromatography (PE:EA=2:1) and 270 mg of (6R,7S,Z)-7-(2-(5-amino-1,2,4-thiadiazol-3-yl)-2-(trityloxyimino)acetamido)-3-(2-((2-(tert-butoxycarbonylamino)ethylthio)methyl)pyridin-3-ylthio)-8-oxo-1-aza-bicyclo[4.2.0]oct-2-ene-2-carboxylic acid was obtained as a white solid in 30% yield.

### Method H

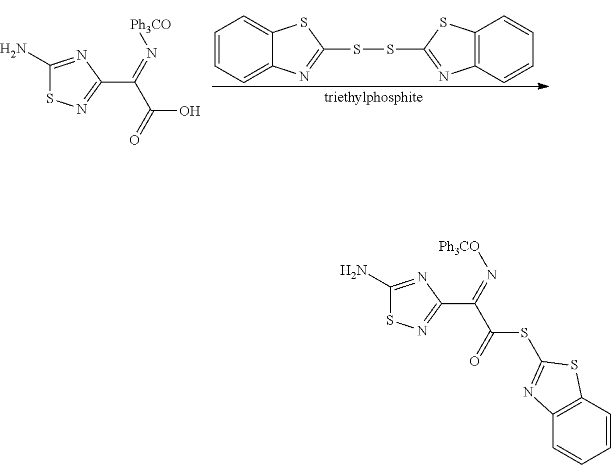

To a solution of (Z)-2-(5-amino-1,2,4-thiadiazol-3-yl)-2-(trityloxyimino)acetic acid (5.4 g, 12 mmol) in anhydrous acetonitrile, TEA (1.265 g, 12.5 mmoL) was added at 0° C. The reaction solution was stirred for 10 min. Disulfide (4.9 g, 14.7 mmoL) was added during 30 min to the reaction solution. Then, a solution of triethyphosphite (3.545 g, 21.35 mmoL) in CH₃CN (30 mL) was added during 30 min. The reaction was stirred at rt for 27 h and filtered. The solid obtained was washed by CH₃CN (50 mL) three times to get (Z)-S-benzo[d]thiazol-2-yl-2-(5-amino-1,2,4-thiadiazol-3-yl)-2-(trityloxyimino)ethanethioate (3.2 g, 46%).

### Method I

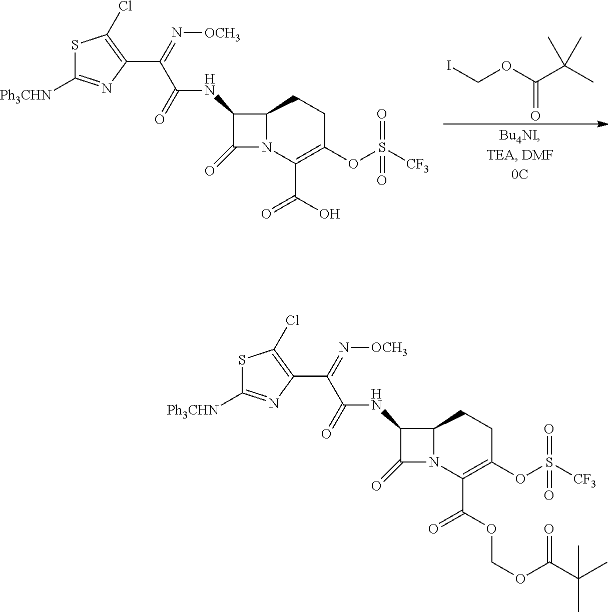

Solvent DMF was added to a solid mixture of (6R,7S,Z)-7-(2-(5-chloro-2-(tritylamino)thiazol-4-yl)-2-(methoxyimino)acetamido)-8-oxo-3-(trifluoromethylsulfonyloxy)-1-aza-bicyclo[4.2.0]oct-2-ene-2-carboxylic acid (2 g, 2.53 mmol) and Bu₄NI (934 mg, 0.25 mmol). Iodomethyl pivalate (12.24 g, 50.6 mmol) and TEA (0.9 mL, 7.6 mmol) were added to the resulting solution at 0° C. in an ice-bath. After 30 min, the ice bath was removed and the solution was stirred for 1 h at rt. Ethyl acetate was added and washed with water twice and brine once, then dried with MgSO₄ and evaporated under reduced pressure. The residue was dissolved in THF, the petroleum ether was and the solution was filtered. 1.2 g pure product in 54% yield was obtained with column chromatography (PE/AE=2:1).

### Method J

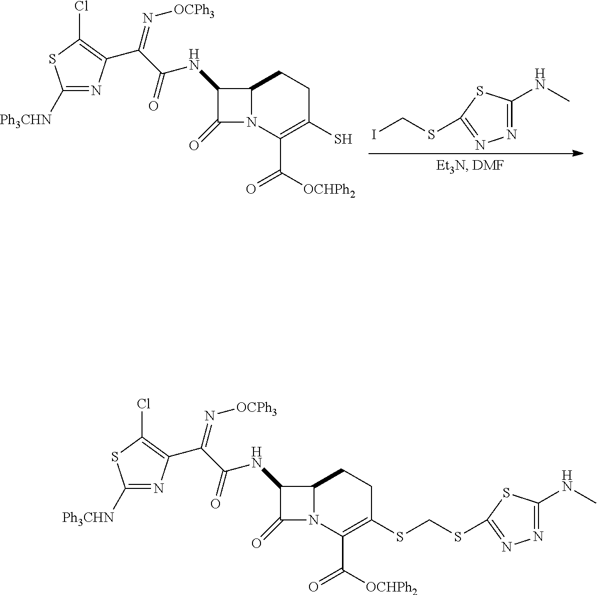

To a solution of (6R,7S,E)-benzhydryl-7-(2-(5-chloro-2-(tritylamino)thiazol-4-yl)-2-(trityloxyimino)acetamido)-3-mercapto-8-oxo-1-aza-bicyclo[4.2.0]oct-2-ene-2-carboxylate (500 mg, 0.47 mmol) in DMF (20 mL) was added TEA (0.8 mL, 0.11 mmol) at rt. After stirring for 30 min, 5-(iodomethylthio)-N-methyl-1,3,4-thiadiazol-2-amine (270 mg, 0.94 mmol) was added to the reaction until no starting material was left by TLC (hexanes/ethyl acetate, 2:1 v/v). The reaction was diluted with ethyl acetate (100 ml) and washed with water (15 ml), brine (15 ml), dried with Na₂SO₄, filtered and evaporated under reduced pressure. The crude product was purified by silica gel column chromatography (hexanes/ethyl acetate, 2:1 v/v) giving (300 mg) pure product as a white solid in 52% yield.

### Example 9

### (6R,7S)-tert-butyl-7-(tert-butoxycarbonylamino)-8-oxo-3-(trifluoromethylsulfonyloxy)-1-aza-bicyclo[4.2.0]oct-2-ene-2-carboxylate

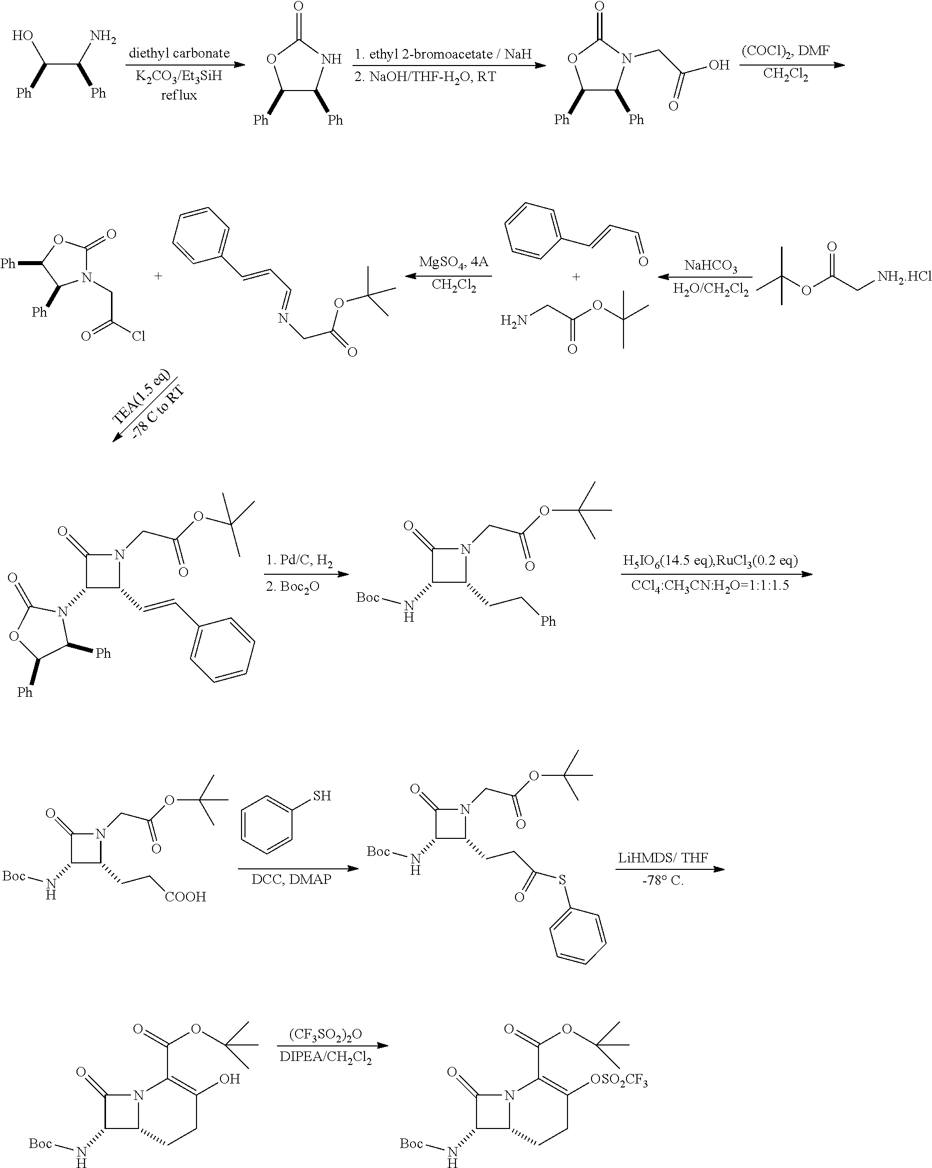

Step 1: A mixture of (1R,2S)-2-amino-1,2-diphenylethanol (4.28 g, 20.0 mmol), K₂CO₃ (0.28 g 2.03 mmol) and diethyl carbonate (20 mL, 166 mmol) was heated under reflux for 16 h. The resulting mixture was washed with water (10 mL) and extracted with CH₂Cl₂ (300 mL). The organic phase was dried MgSO₄, filtered and concentrated. The residue was recrystallized form toluene to give the desired compound (4S,5R)-4,5-diphenyloxazolidin-2-one as white solid. Yield 88%, ESI-MS: 240.1 [M⁺]

Step 2: NaH (0.66 g, 60% mineral oil dispersion, 20.8 mmol) was placed in a three necked flask under argon and washed with anhydrous hexane (15 mL). After addition of THF (50 mL) to NaH, a solution of (4S,5R)-4,5-diphenyloxazolidin-2-one in THF was added to the suspension and the mixture was stirred for 2 h at rt. Then, ethyl bromoacetate was added dropwise in a period of 30 min, and the mixture was stirred for 30 min. The reaction was quenched with water (100 mL) and extracted with CH₂Cl₂. The combined extracts were washed with water, dried over MgSO₄, filtered and concentrated. The residue was purified by silica gel column chromatography to give ethyl-2-((4S,5R)-2-oxo-4,5-diphenyloxazolidin-3-yl)acetate.

A THF (20 mL) solution of ethyl-2-((4S,5R)-2-oxo-4,5-diphenyloxazolidin-3-yl)acetate was added to a solution of KOH (2.99 g, 53.3 mmol) in H₂O/MeOH/THF (35 mL, H₂O:MeOH:THF=3:3:8) and the mixture was stirred for 2 h at rt. Then, 1 M aq HCl (100 mL) was added to the mixture. The desired product was extracted with Et₂O (3×100 mL) and the combined extracts were washed with sat. aq NaCl (50 mL), dried over MgSO₄, filtered and concentrated. The residue was recrystallized from toluene to give 2-((4S,5R)-2-oxo-4,5-diphenyloxazolidin-3-yl)acetic acid yield 83%. ESI-MS: 298.1 [M⁺].

Step 3: Glycine t-butyl ester hydrochloride (125 g, 0.75 mol) was treated with 10 N aqueous sodium hydroxide (180 mL) and extracted with dichloromethane. The dichloromethane solution was back washed with saturated aqueous NaCl, dried by sodium sulfate, filtered and concentrated in vacuum to get the glycine-butyl ester (60 g). Tert-butyl-2-aminoacetate (31.5 g, 0.24 mol) in dichloromethane was treated sequentially with 1 equivalent of cinnamaldehyde (26.4 g, 0.2 mol) and a desiccating agent, such as magnesium sulfate (70 g), in the amount of about 2 grams of desiccating agent per gram of starting amino acid ester or amide. The reaction was stirred at ambient temperature until all of the reactants were consumed as measured by thin layer chromatography. The reactions were typically complete after 3 h. The reaction mixture was then filtered and the filter cake was washed with dichloromethane. The filtrate was concentrated under reduced pressure to provide the desired imine that was used as is in the subsequent step.

Step 4: 2-((4S,5R)-2-oxo-4,5-diphenyloxazolidin-3-yl)acetic acid (5.95 g, 20 mmol) was dissolved in CH₂Cl₂. Then, DMF (0.04 mL, 0.6 mmol) was added, followed by COCl)₂ (2.6 mL, 30 mmol). The reaction mixture was stirred for 1.5 h at rt and concentrated. The product was used for next step without further purification. Triethylamine (4.18 mL, 30.0 mmol) was added at −78° C. to a solution of the acid chloride (6.32 g, 20.0 mmol) in dry methylene chloride (100 mL). After 20 min, a solution of the imine (4.91 g, 20.0 mmol) in dry CH₂Cl₂ (50 mL) was added dropwise at the same temperature. The cooling bath was removed and the resulting mixture was stirred under nitrogen atmosphere at 0° C. for 2 h. Then, the reaction mixture was successively washed with water 100 mL, 1 N HCl (50 mL), saturated aqueous solution of NaHCO₃ (100 mL). The organic layer was dried over MgSO₄, filtered and concentrated to afford a crude material, which was washed by little CH₃OH to afford a white solid tert-butyl-2-((3S,4R)-2-oxo-3-((4S,5R)-2-oxo-4,5-diphenyloxazolidin-3-yl)-4-((E)-styryl)azetidin-1-yl)acetate. ESI-MS: 525.1 [M⁺]. ¹H NMR (300 MHz, DMSO-d₆): δ 7.46-6.91 (m, 15H), 6.80 (d, 15.6Hz, 1H), 6.37 (dd, 8.1/15.9 Hz, 1H), 5.93 (d, 8.1Hz, 1H), 5.28 (d, 8.1Hz, 1H), 4.60-4.50 (m, 2H), 4.60 (d, 17.7 Hz, 1H), 3.77 (d, 18.0 Hz, 1H), 1.358 (s, 9H).

Step 5: Pearlman's catalyst (2 g) and di-tert-butyl dicarbonate (6.5 g, 30 mmol) were added successively to a solution of the corresponding tert-butyl-2-((3S,4R)-2-oxo-3-((4S,5R)-2-oxo-4,5-diphenyloxazolidin-3-yl)-4-((E)-styryl)azetidin-1-yl)acetate (1 mmol) in THF (30 mL). The resulting mixture was stirred at rt under a hydrogen atmosphere (120 psi) for 48 h. Then, the mixture was filtered through Celite. After evaporation of the filtrate under reduced pressure, the resulting crude was crystallization by methanol to give 2-((3S,4R)-3-(tert-butoxycarbonylamino)-2-oxo-4-phenethylazetidin-1-yl) acetate (2.63 g, 65%). ESI-MS: 405.2 [M⁺]

Step 6: To a mixture of the tert-butyl-2-((3S,4R)-3-(tert-butoxycarbonylamino)-2-oxo-4-phenethylazetidin-1yl)acetate (2.1 g, 5.19 mmol) in carbon tetrachloride (30 mL), acetonitrile (30 mL), and water (45 mL) was added at rt periodic acid (17.24 g, 75.26 mmol). The biphasic mixture was stirred until both phases became clear, and ruthenium trichloride hydrate (236 mg, 1.05 mmol) was added. Stirring was continued until no starting material was detected by TLC (4 h). The reaction mixture was cooled down to 0° C., and diethyl ether (300 mL) was added with vigorous stirring for 10 min. The organic phase was separated and the aqueous layer extracted with diethyl ether (2×150 mL). The combined organic layers were washed with brine (100 mL), dried, filtered, and concentrated. The resulting crude was purified by chromatography (CH₂Cl₂:EA=2:1) to give 3-((2R,3S)-1-(2-tert-butoxy-2-oxoethyl)-3-(tert-butoxycarbonylamino)-4-oxoazetidin-2-yl)propanoic acid (1.2 g, 62%). ESI-MS: 373.2 [M⁺]

Step 7: To a cold (0° C.) solution of (6.8 g, 16.83 mmol) of 3-((2R,38)-1-(2-tert-butoxy-2-oxoethyl)-3-(tert-butoxycarbonylamino)-4-oxoazetidin-2-yl)propanoic acid in 300 mL of methylene chloride maintained under nitrogen were added 103.7 mg (0.85 mmol) of dimethylaminopyridine, thiophenol (2.32 g, 21.04 mmol), and dicyclohexylcarbodiimide (DCC) (4.34 g, 21.04 mmol). The mixture was stirred at 0° C. for 10 minutes and at rt for 6 h. The mixture was poured into 400 mL of methylene chloride and the mixture washed with an aqueous sodium bicarbonate solution (50% of saturated), with 1 M hydrochloric acid, and with saturated sodium bicarbonate solution. The organic phase was dried over sodium sulfate, filtered and evaporated to dryness to yield the title compound as partly crystalline oil. The resulting crude was purified by chromatography (CH₂Cl₂:EA=4:1) to give tert-butyl-2-((3S,4R)-3-(tert-butoxycarbonylamino)-2-oxo-4-(3-oxo-3-(phenylthio)propyl)azetidin-1-yl)acetate (7.5 g, 89%). ESI-MS: 465.2 [M⁺]

Step 8: To a solution of 18.12 g (39 mmol) of tert-butyl-2-((3S,4R)-3-(tert-butoxycarbonylamino)-2-oxo-4-(3-oxo-3-(phenylthio)propyl)azetidin-1yl)acetate in 300 mL of anhydrous THF and maintained under argon at −78° C. was added 156 mL (156 mmol) of lithium hexamethyldisilazane (1M/L) (also maintained under argon at −78° C.). After about 6 h, the mixture was poured into 1000 mL of aqueous ammonium chloride (50% of saturation) and the pH was adjusted to 3 with 1 M HCl aqueous. The acidified mixture was extracted three times with 800 mL of portions of methylene chloride. The extracts was combined, washed with brine, dried over sodium sulfate, filtered and concentrated by evaporation. The residue was initially chromatographed over silica using hexane-ethyl acetate (ca 3:1, v/v), followed by a (2:1, v/v). mixture of the same solvents for elution of the product. The desired fraction was evaporated to dryness to provide (6R,7S)-tert-butyl-7-(tert-butoxycarbonylamino)-3-hydroxy-8-oxo-1-aza-bicyclo-[4.2.0]oct-2-ene-2-carboxylate (10.2 g, 74%). ESI-MS: 377.1 [M⁺]

Step 9: A CH₂Cl₂ solution of trifluoromethanesulfonic anhydride (338.4 mg, 1.2 mmol) was rapidly added to a solution of (6R,7S)-tert-butyl-7-(tert-butoxycarbonylamino)-3-hydroxy-8-oxo-1-aza-bicyclo[4.2.0]oct-2-ene-2-carboxylate (354 mg, 1 mmol) and DIPEA (193 mg, 1.5 mmol) in CH₂Cl₂ (5 mL) at −40° C. After 15 min, the reaction mixture was poured into a saturated aqueous solution of NaHCO₃ (10 mL). The resulting mixture was extracted with CH₂Cl₂ (3×20 mL). The extracts were combined, washed with brine (1×5 mL), dried (MgSO₄), filtered and concentrated to give 438 mg (91%) of (6R,7S)-tert-butyl-7-(tert-butoxycarbonylamino)-8-oxo-3-(trifluoromethylsulfonyloxy)-1-aza-bicyclo[4.2.0]oct-2-ene-2-carboxylate as a white solid. ESI-MS: 486.2 [M⁺]. ¹H NMR (300 MHz, DMSO-d₆): δ 7.71 (d, 6.9Hz, 1H), 5.20 (dd, 4.2/6.9 Hz, 1H), 3.85 (dd, 4.2/8.4 Hz, 1H), 2.62 (d, 2.7 Hz, 2H), 1.90-1.80 (m, 2H), 1.46 (s, 9H), 1.38 (s, 9H)

### Example 10

### (Z)-S-benzo[d]thiazol-2-yl-2-(5-amino-1,2,4-thiadiazol-3-yl)-2-(trityloxyimino)ethanethioate

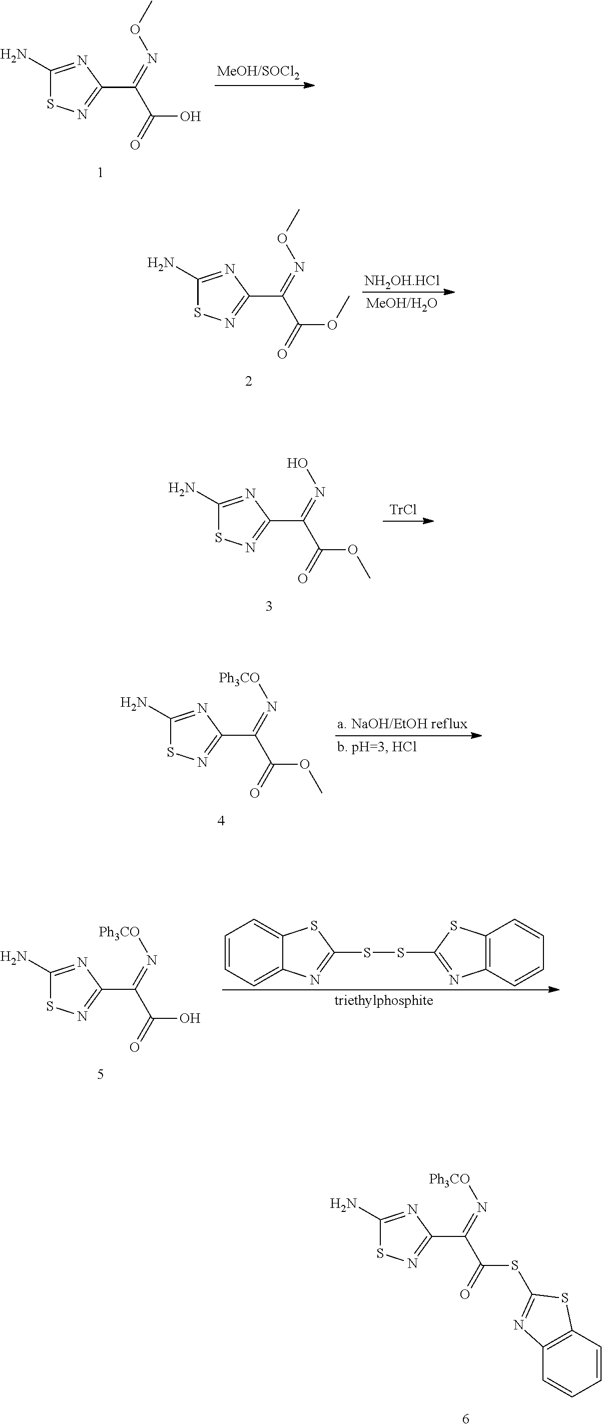

Step 1: SOCl₂ (200 mL, 2.74 mol) was slowly added to methanol (400 mL) during 1.5 h at 0° C. Then, (Z)-2-(5-amino-1,2,4-thiadiazol-3-yl)-2-(methoxyimino)acetic acid 1 (61 g, 0.30 mol) was added in one portion and the reaction mixture was stirred for 24 h at 70° C. Concentration gave a white solid which was partitioned between ethyl acetate (500 mL×3) and water (200 mL). The organic phase was washed (saturated NaHCO₃, water), dried on Na₂SO₄ and concentrated to give 56 g (Z)-methyl-2-(5-amino-1,2,4-thiadiazol-3-yl)-2-(methoxyimino)acetate 2 as a white solid in 85% yield. This product was used without further purification.

Step 2: A solution of (Z)-methyl-2-(5-amino-1,2,4-thiadiazol-3-yl)-2-(methoxyimino)acetate 2 (60 g, 0.28 mol) and NH₂OH.HCl (140 g, 1.98 mol) in methanol (400 mL) and H₂O (200 mL) was stirred at 100° C. for 24 h. Concentration gave a yellow syrup which was partitioned between ethyl acetate (1 L) and water (400 mL). The aqueous layer was extracted with ethyl acetate (2×1 L). The organic phase was dried on NaSO₄, filtered and concentrated to dry. The crude solid was crystallized from DCM/PE (20:1, 1 L) 5 times, the product was collected and 20 g of (Z)-methyl-2-(5-amino-1,2,4-thiadiazol-3-yl)-2-(hydroxyimino)acetate 3 was obtained as a white solid in 45% yield. The product was used without further purification.

Step 3: To a solution of (Z)-methyl-2-(5-amino-1,2,4-thiadiazol-3-yl)-2-(hydroxyimino)acetate 3 (10 g, 0.05 mol) in 50 mL THF at 0° C. was added 5.5 g TEA, stirred 10 minutes. Then, 14 g of trityl chloride was added in at 0° C. and the reaction solution was stirred at this temperature for 2 h. Concentration gave a white solid which was partitioned between ethyl acetate (500 mL) and water (200 mL). The aqueous layer was extracted with ethyl acetate (2×500 mL). The organic phase was washed with 1% NaOH aqueous solution (200 mL) three times. The organic phase was dried over NaSO₄, filtered and concentrated to dry. The solid obtained was crystallized from petroleum ether (1 L). The product was collected and (Z)-methyl-2-(5-amino-1,2,4-thiadiazol-3-yl)-2-(trityloxyimino)acetate 4 (15 g) was obtained as a white solid in 68% yield. The product was used without further purification.

Step 4: A solution of (Z)-methyl-2-(5-amino-1,2,4-thiadiazol-3-yl)-2-(trityloxyimino)acetate 4 (15 g, 33.8 mmol) in 80 mL of 2.5 M NaOH and 40 mL ethanol was heated to gentle reflux for 2 h. The reaction solution was cooled down and THF (40 mL) was added in. Then, adjusted reaction solution to pH=3 by 5% aq. HCl. The whole reaction solution was extracted with ethyl acetate (100 mL). The organic phase was dried on Na₂SO₄, filtered and concentrated. The crude (Z)-2-(5-amino-1,2,4-thiadiazol-3-yl)-2-(trityloxyimino)acetic acid 5 (12 g, 28.0 mmol) was obtained as white solid in 70% yield. The product was used without further purification.

Step 5: To a solution of (Z)-2-(5-amino-1,2,4-thiadiazol-3-yl)-2-(trityloxyimino)acetic acid 5 (5.4 g, 12 mmol) in anhydrous acetonitrile TEA (1.265 g, 12.5 mmoL) was added in at 0° C. The reaction solution was stirred for 10 min. Disulfide (4.9 g, 14.7 mmoL) was added during 30 min to the reaction solution. Then, a solution of triethyphosphite (3.545 g, 21.35 mmoL) in CH₃CN (30 mL) was added during 30 min. The reaction was stirred at rt for 27 h and filtered. The solid obtained was washed by CH₃CN (50 mL) three times to get (Z)-S-benzo[d]thiazol-2-yl-2-(5-amino-1,2,4-thiadiazol-3-yl)-2-(trityloxyimino)ethanethioate 6 (3.2 g, 46%).

### Example 11

### (Z)-S-benzo[d]thiazol-2-yl-2-(5-amino-1,2,4-thiadiazol-3-yl)-2-(methoxyimino)ethanethioate

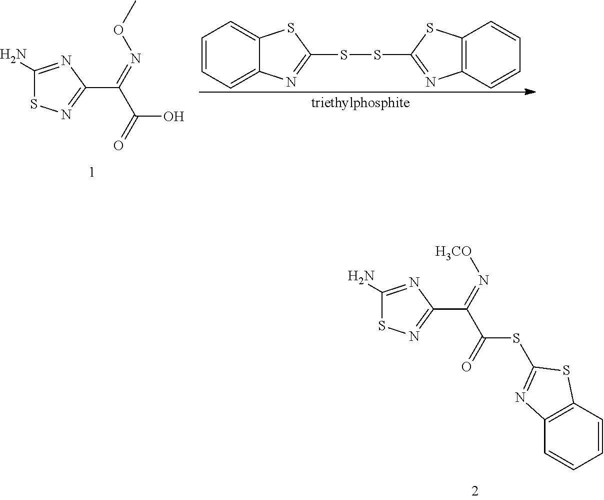

(Z)-S-benzo[d]thiazol-2-yl-2-(5-amino-1,2,4-thiadiazol-3-yl)-2-(methoxyimino)ethanethioate 2 was prepared from (Z)-2-(5-amino-1,2,4-thiadiazol-3-yl)-2-(methoxyimino)acetic acid 1 in 40% yield by Method H. The product was used without further purification. ESI-MS: 352.4 [M+H].

### Example 12

### (Z)-S-benzo[d]thiazol-2-yl-2-(5-chloro-2-(tritylamino)thiazol-4-yl)-2-(trityloxyimino)ethanethioate

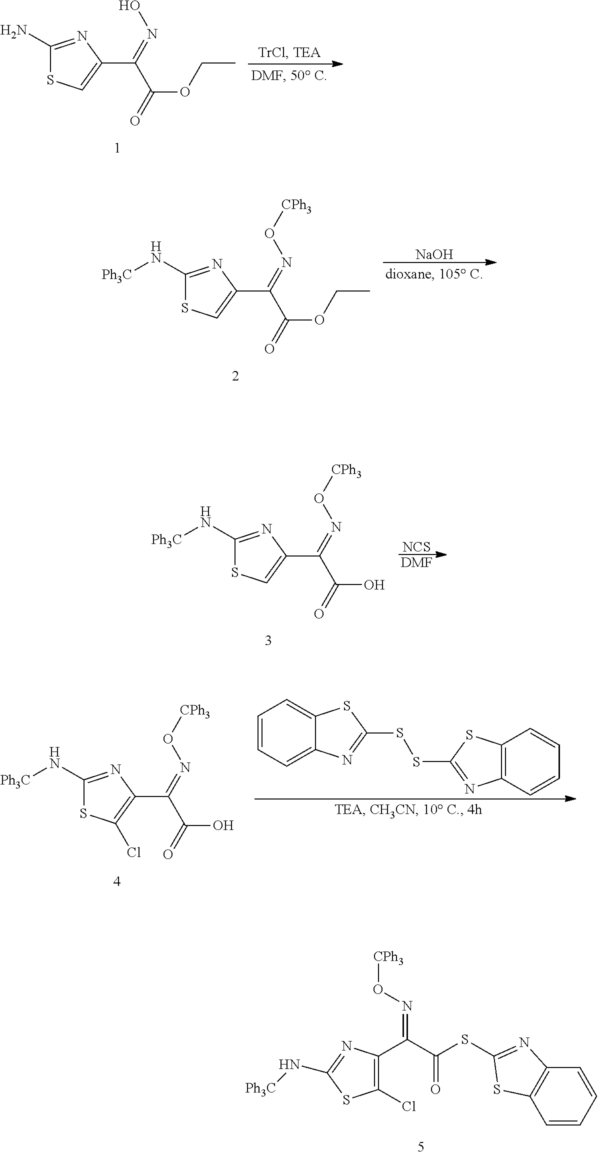

Step 1: To a solution of (Z)-ethyl-2-(2-aminothiazol-4-yl)-2-(hydroxyimino)acetate 1 (21.5 g, 0.1 mol) in 100 mL DMF, Et₃N (30.6 mL, 0.22 mol) was added. Then, TrCl (64 g, 0.22 mol) was added during 20 minutes, the reaction solution was stirred at 50° C. for 48 h until LC-MS indicated the reaction was over. The reaction solution was slowly poured into water (600 mL). (Z)-ethyl-2-(2-(tritylamino)thiazol-4-yl)-2-(trityloxyimino)acetate 2 was obtained as a white solid in 85% yield. The resulting product was used without further purification.

Step 2: To a solution of (Z)-ethyl-2-(2-(tritylamino)thiazol-4-yl)-2-(trityloxyimino)acetate 2 (14.5 g 20.7 mmol) in H₂O (10 mL) and dioxane (80 mL), NaOH aqueous (1.7 g, 41.4 mmol) was added in. The reaction solution was refluxed for 24 h until LC-MS indicated there no starting material existed. Then, water (200 mL) was added under stirring. The mixture was cooled down to a temperature between 0° C. to 5° C. under stirring and the precipitated solid was filtered, washed by dioxane and dried under vacuum to give 10.0 g of (Z)-2-(2-(tritylamino)thiazol-4-yl)-2-(trityloxyimino)acetic acid 3 in 72% yield as a white solid. The resulting product was used without further purification.

Step 3: To a solution of (Z)-2-(2-(tritylamino)thiazol-4-yl)-2-(trityloxyimino)acetic acid 3 (67 g, 0.1 mol) in 400 mL DMF, NCS (25 g, 0.19 mol) was added during 5 minutes. The reaction mixture was stirred at 0° C. for 3 h. To the reaction solution, 600 mL of water was added and the whole reaction solution was extracted with 300 mL of ethyl acetate. The organic layer was washed with 200 mL of saturated aqueous sodium chloride solution, dried over anhydrous magnesium sulfate, filtered and concentrated to dry. The residue was crystallized from ethyl acetate to give crude (Z)-2-(5-chloro-2-(tritylamino)thiazol-4-yl)-2-(trityloxyimino)acetic acid as a white solid. The resulting product was purified by column chromatography (50% EtOAc in petroleum ester) to give (Z)-2-(5-chloro-2-(tritylamino)thiazol-4-yl)-2-(trityloxyimino)acetic acid 4 as a white solid (76 g, 80%).

Step 4: (Z)-S-benzo[d]thiazol-2-yl-2-(5-chloro-2-(tritylamino)thiazol-4-yl)-2-(trityloxyimino)ethanethioate 5 was prepared from (Z)-2-(5-chloro-2-(tritylamino)thiazol-4-yl)-2-(trityloxyimino)acetic acid 4 according to Method H. The resulting product was purified by column chromatography (20% DCM in petroleum ester) in 20% yield as a white solid. ESI-MS: 8653.2 [M+H]. The compound may be used in methods similar to those of Methods A-I.

### Example 13

### (Z)-S-benzo[d]thiazol-2-yl-2-(2-amino-5-chlorothiazol-4-yl)-2-(methoxyimino)ethanethioate

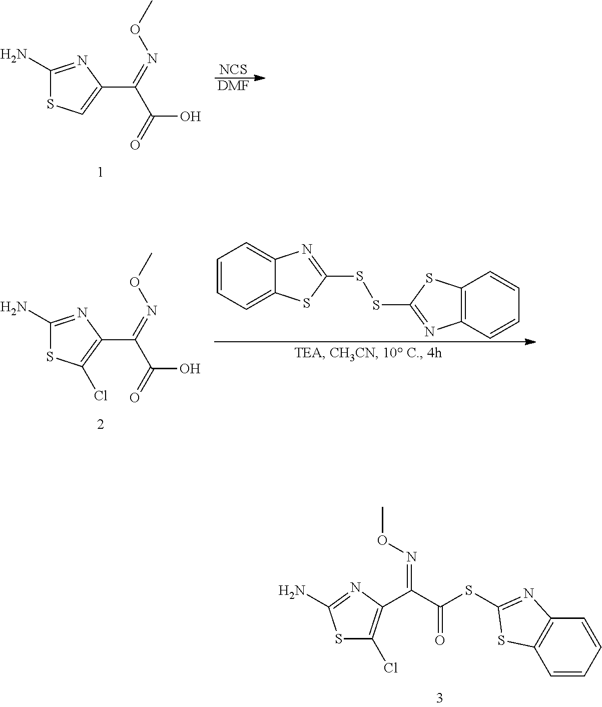

Step 1: (Z)-ethyl-2-(2-amino-5-chlorothiazol-4-yl)-2-(methoxyimino)acetate 2 was prepared from (Z)-ethyl-2-(2-aminothiazol-4-yl)-2-(methoxyimino)acetate 1 as set forth in Example 12. The resulting product was purified by column chromatography (50% EtOAc in petroleum ester) in 80% yield.

Step 2: (Z)-S-benzo[d]thiazol-2-yl-2-(2-amino-5-chlorothiazol-4-yl)-2-(methoxyimino)ethanethioate 3 was prepared from (Z)-ethyl-3-((Z)-amino(methylthio)methyleneamino)-2-(methoxyimino)propanoate by following Method H. The resulting product (Z)-S-benzo[d]thiazol-2-yl-2-(2-amino-5-chlorothiazol-4-yl)-2-(methoxyimino)ethanethioate 3 was purified by column chromatography (20% DCM in petroleum ester as eluent) in 16% yield as a white solid. ESI-MS: 345.9 [M+H]. The compound may be used in methods similar to those of Methods A-I.

### Example 14

### Iodomethyl Pivalate

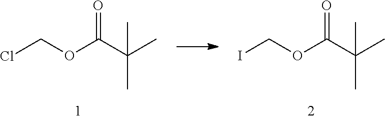

Sodium iodide (67.5 g, 448.2 mmol) was added in one portion to a solution of 2,2-dimethylpropanoic acid chloromethyl ester 1 (45 g, 300 mmol) in 150 mL dry acetonitrile at rt under nitrogen. The heterogeneous reaction was stirred at rt for 18 h, then filtered and concentrated in vacuo. The residue was partitioned between ethyl acetate and 5% sodium bisulfite. The organic layer was washed with 5% sodium bisulfite and water, then dried over Mg₂SO₄, filtered and evaporated to get 56 g of pure product 2 with 77% yield. ¹H NMR (400 MHz, chloroform-d): 1.20 (s, 9H), 5.93 (s, 2H).

### Example 15

### Pyridin-4-ylmethanethiol

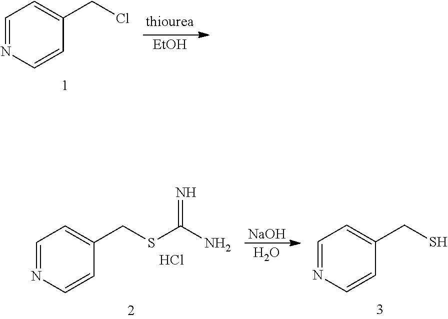

Step 1: To a solution of 4-(chloromethyl)pyridine 1 (10 g, 66 mmol) in 60 mL ethanol, thiourea (5.8 g, 77 mmol) was added, and then heated at reflux for 1.5 h. After cooling, the precipitate was filtered, and washed with ether and dried to give pyridin-4-ylmethyl carbamimidothioate 2 (13.4g) as a white solid in 98% yield.

Step 2: To a solution of sodium hydroxide (1.28 g) in water (10 mL) pyridin-4-ylmethyl carbamimidothioate 2 (2.0 g) was added, and then heated to 70° C. for 30 min. After cooling, acidify with hydrogen chloride solution (4 M) to pH=7.0. Extracted with DCM, dried over Na₂SO₄ and concentrated in vacuum. The residue was purified by column chromatography to afford pyridin-4-ylmethanethiol 3 in 47% yield as a yellow solid. ESI-MS: 126.0 [M+H].

### Example 16

### (6R,7S,Z)-tert-Butyl-7-(tert-butoxycarbonylamino)-3-(2-(4-methylthiazol-5-yl)vinyl)-8-oxo-1-aza-bicyclo[4.2.0]oct-2-ene-2-carboxylate

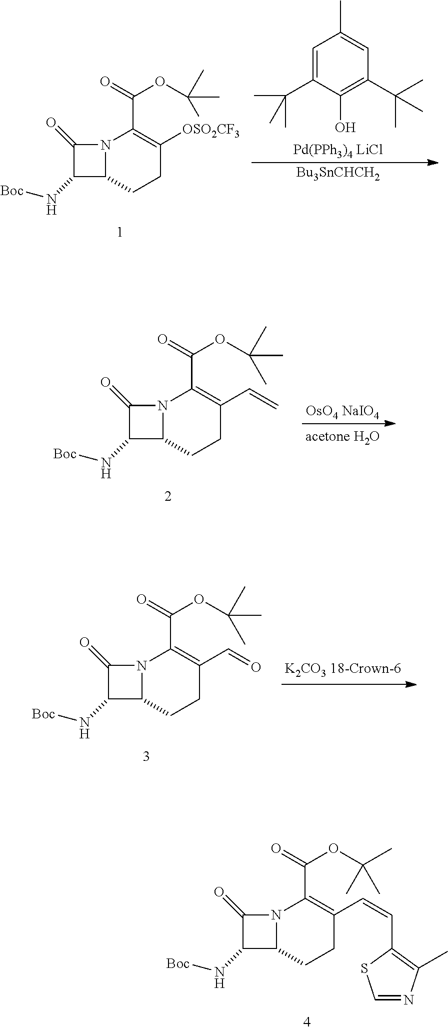

Step 1: (6R,7S)-tert-butyl-7-(tert-butoxycarbonylamino)-8-oxo-3-(trifluoromethylsulfonyloxy)-1-aza-bicyclo[4.2.0]oct-2-ene-2-carboxylate 1 (400 mg) and Pd(PPh₃)₄ (38 mg, 0.04 eq), LiCl (104 mg, 3 eq), 2,6-di-tert-butyl-4-methylphenol (15 mg), and Bu₃SnCHCH₂ (286 mg, 1.1 eq) were dissolved in dry dioxane (15 mL) and refluxed for 3 h at 100° C. After removal of the solvent, the black residue was extracted with CH₂Cl₂ and washed with water, the organic layer was dried over MgSO₄, then concentrated and purified through silica gel. (6R,7S)-tert-butyl-7-(tert-butoxycarbonylamino)-8-oxo-3-vinyl-1-aza-bicyclo[4.2.0]oct-2-ene-2-carboxylate 2 was obtained as a white solid (210 mg, 70%).

Step 2: To a solution of (6R,7S)-tert-butyl-7-(tert-butoxycarbonylamino)-8-oxo-3-vinyl-1aza-bicyclo[4.2.0]oct-2-ene-2-carboxylate 2 (182 mg) in H₂O/acetone (1:3, 10 mL) was added NaI0₄ (236 mg), then OsO₄/H₂O (3 mL) was added and the solution was kept at rt for 1 h. When LC-MS showed the reaction was over, the solution was slowly added to Na₂CO₃. After removal of the solvent, the black residue was extracted with CH₂Cl₂ and washed with water, the organic layer was dried over MgSO₄, then concentrated and purified through silica gel. (6R,7S)-tert-butyl-7-(tert-butoxycarbonylamino)-3-formyl-8-oxo-1-aza-bicyclo[4.2.0]oct-2-ene-2-carboxylate 3 was obtained as a white solid (55 mg, 30%).

Step 3: K₂CO₃ (90 mg) and 18-Crown-6 (6 mg, 23 μmol) were added to a stirred solution of (6R,7S)-tert-butyl-7-(tert-butoxycarbonylamino)-3-formyl-8-oxo-1-aza-bicyclo[4.2.0]oct-2-ene-2-carboxylate 3 (200 mg, 546 mmol) and triphenylpyridin-4-ylmethylphosphonium bromide (300 mg, 600 mmol) in anhydrous DCM (30 mL). After 3 h, more K₂CO₃ (23 mg, 0.17 mmol) was added and stirring was continued for an additional 3 h. The mixture was partitioned between DCM (25 mL) and H₂O (10 mL). The organic phase was washed with H₂O (10 mL), before being dried over MgSO₄. Filtration, solvent evaporation, and purification by RP-HPLC gave 90 mg of (6R,7S,Z)-tert-butyl-7-(tert-butoxycarbonylamino)-3-(2-(4-methylthiazol-5-yl)vinyl)-8-oxo-1aza-bicyclo[4.2.0]oct-2-ene-2-carboxylate 4 as a white solid in 45% yield.

### Example 17

### (6R,7S,Z)-7-(2-(2-amino-5-chlorothiazol-4-yl)-2-(hydroxyimino)acetamido)-8-oxo-3-(pyridin-4-ylmethylthio)-1-aza-bicyclo[4.2.0]oct-2-ene-2-carboxylic acid

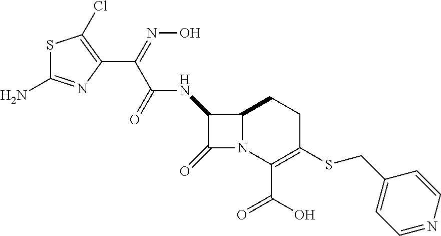

Step 1: (6R,7S,Z)-7-(2-(5-chloro-2-(tritylamino)thiazol-4-yl)-2-(trityloxyimino)acetamido)-8-oxo-3-(trifluoromethylsulfonyloxy)-1-aza-bicyclo[4.2.0]oct-2-ene-2-carboxylic acid was prepared from (6R,7S)-7-amino-8-oxo-3-(trifluoromethylsulfonyloxy)-1-aza-bicyclo[4.2.0]oct-2-ene-2-carboxylic acid in 90% yield by following Method B. The resulting product (white powder) was used without further purification.

Step 2: (6R,7S,Z)-benzhydryl-7-(2-(5-chloro-2-(tritylamino)thiazol-4-yl)-2-(trityloxyimino)acetamido)-8-oxo-3-(trifluoromethylsulfonyloxy)-1-aza-bicyclo[4.2.0]oct-2-ene-2-carboxylate was prepared from (6R,7S,Z)-7-(2-(5-chloro-2-(tritylamino)thiazol-4-yl)-2-(trityloxyimino)acetamido)-8-oxo-3-(trifluoromethylsulfonyloxy)-1-aza-bicyclo[4.2.0]oct-2-ene-2-carboxylic acid in 97% yield by following Method C. The resulting product (white powder) was used without further purification.

Step 3: (6R,7S,Z)-benzhydryl-7-(2-(5-chloro-2-(tritylamino)thiazol-4-yl)-2-(trityloxyimino)acetamido)-8-oxo-3-(pyridin-4-ylmethylthio)-1-aza-bicyclo[4.2.0]oct-2-ene-2-carboxylate was prepared from (6R,7S,Z)-benzhydryl-7-(2-(5-chloro-2-(tritylamino)thiazol-4-yl)-2-(trityloxyimino)acetamido)-8-oxo-3-(trifluoromethylsulfonyloxy)-1-aza-bicyclo[4.2.0]oct-2-ene-2-carboxylate by following Method D. The resulting product was purified by column chromatography (eluting solvent: PE:EA=3:2) in 39.5% yield as a slight yellow solid.

Step 4: (6R,7S,Z)-7-(2-(2-amino-5-chlorothiazol-4-yl)-2-(hydroxyimino)acetamido)-8-oxo-3-(pyridin-4-ylmethylthio)-1-aza-bicyclo[4.2.0]oct-2-ene-2-carboxylic acid 1 was prepared from (6R,7S,Z)-benzhydryl-7-(2-(5-chloro-2-(tritylamino)thiazol-4-yl)-2-(trityloxyimino)acetamido)-8-oxo-3-(pyridin-4-ylmethylthio)-1-aza-bicyclo[4.2.0]oct-2-ene-2-carboxylate as a slight yellow solid in 27.8% yield by following Method E. ESI-MS: 509.0 [M+H].

### Example 18

### (6R,7S,Z)-7-(2-(2-amino-5-chlorothiazol-4-yl)-2-(hydroxyimino)acetamido)-3-(benzylthio)-8-oxo-1-aza-bicyclo[4.2.0]oct-2-ene-2-carboxylic acid

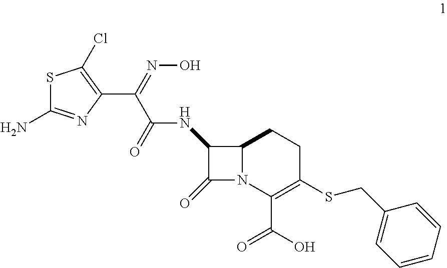

Step 1: (6R,7S,Z)-7-(2-(5-chloro-2-(tritylamino)thiazol-4-yl)-2-(trityloxyimino)acetamido)-8-oxo-3-(trifluoromethylsulfonyloxy)-1-aza-bicyclo[4.2.0]oct-2-ene-2-carboxylic acid was prepared from (6R,7S)-7-amino-8-oxo-3-(trifluoromethylsulfonyloxy)-1-aza-bicyclo[4.2.0]oct-2-ene-2-carboxylic acid in 90% yield by following Method B. The resulting product (white powder) was used without further purification.

Step 2: (6R,7S,Z)-benzhydryl-7-(2-(5-chloro-2-(tritylamino)thiazol-4-yl)-2-(trityloxyimino)acetamido)-8-oxo-3-(trifluoromethylsulfonyloxy)-1-aza-bicyclo[4.2.0]oct-2-ene-2-carboxylate was prepared from (6R,7S,Z)-7-(2-(5-chloro-2-(tritylamino)thiazol-4-yl)-2-(trityloxyimino)acetamido)-8-oxo-3-(trifluoromethylsulfonyloxy)-1-aza-bicyclo[4.2.0]oct-2-ene-2-carboxylic acid in 97% yield by following Method C. The resulting product (white powder) was used without further purification.

Step 3: (6R,7S,Z)-benzhydryl-3-(benzylthio)-7-(2-(5-chloro-2-(tritylamino)thiazol-4-yl)-2-(trityloxyimino)acetamido)-8-oxo-1aza-bicyclo[4.2.0]oct-2-ene-2-carboxylate was prepared from (6R,7S,Z)-benzhydryl-7-(2-(5-chloro-2-(tritylamino)thiazol-4-yl)-2-(trityloxyimino)acetamido)-8-oxo-3-(trifluoromethylsulfonyloxy)-1-aza-bicyclo[4.2.0]oct-2-ene-2-carboxylate by following Method D. The resulting product was purified by column chromatography (eluting solvent: PE:EA=3:2) in 39.5% yield as a slight yellow solid.

Step 4: (6R,7S,Z)-7-(2-(2-amino-5-chlorothiazol-4-yl)-2-(hydroxyimino)acetamido)-3-(benzylthio)-8-oxo-1-aza-bicyclo[4.2.0]oct-2-ene-2-carboxylic acid 1 was prepared from (6R,7S,Z)-benzhydryl-3-(benzylthio)-7-(2-(5-chloro-2-(tritylamino)thiazol-4-yl)-2-(trityloxyimino)acetamido)-8-oxo-1aza-bicyclo[4.2.0]oct-2-ene-2-carboxylate as a slight yellow solid in 27.8% yield by following Method E. ESI-MS: 508.0 [M+H].

### Example 19

### (6R,7S,E)-benzhydryl 7-(2-(5-chloro-2-(tritylamino)thiazol-4-yl)-2-(trityloxyimino)acetamido)-3-mercapto-8-oxo-1-aza-bicyclo[4.2.0]oct-2-ene-2-carboxylate

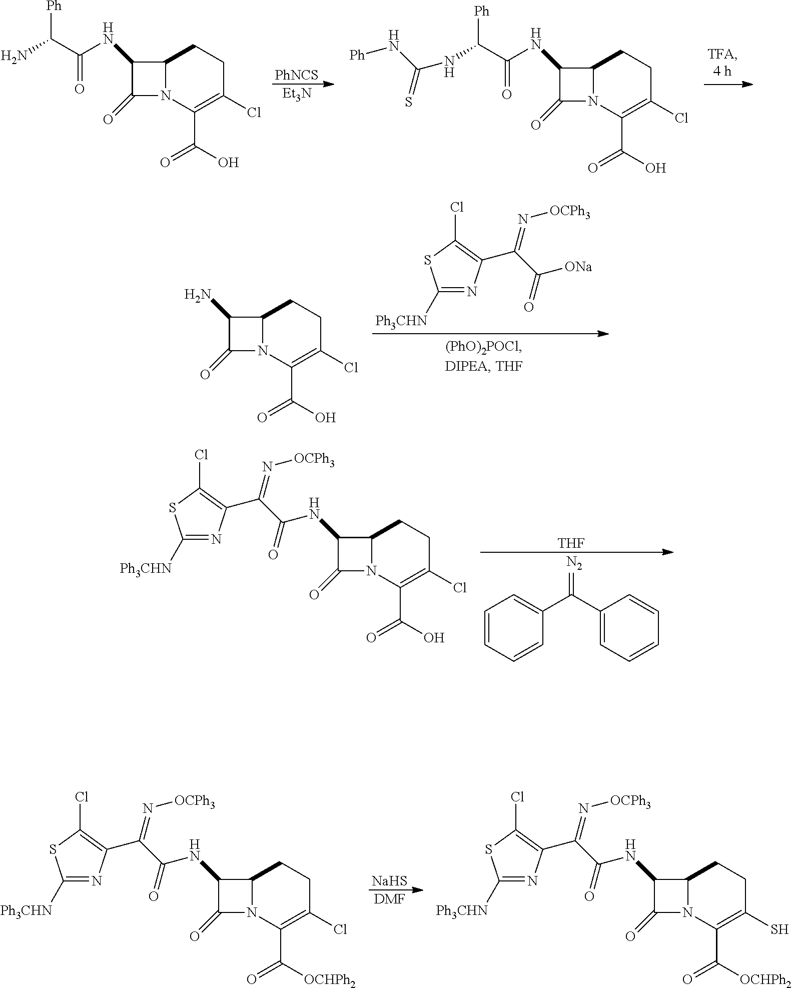

Step 1: To a solution of (6R,7S)-7-((R)-2-amino-2-phenylacetamido)-3-chloro-8-oxo-1-aza-bicyclo[4.2.0]oct-2-ene-2-carboxylic acid (1.0 g, 2.86 mmol) in DMF (10 mL), TEA (0.8 mL, 5.72 mmol) was added and the reaction mixture was stirred for 30 min at 20° C. Phenylisotiocyanate (424 mg, 3.14 mmol) was added and the mixture was stirred for 24 h. The reaction was then poured into 2-methoxy-2-methylpropane (100 ml). The mixture was stirred for 12 h and filtered. The solid product was washed with 2-methoxy-2-methylpropane (3×20 ml) and dried at 80° C. for 2 days to give (1.33 g) of (6R,7S)-3-chloro-8-oxo-7-((R)-2-phenyl-2-(3-phenylthioureido)acetamido)-1-aza-bicyclo[4.2.0]oct-2-ene-2-carboxylic acid in 96% yield which was used for next step without further purification.

Step 2: (6R,7S)-3-chloro-8-oxo-7-((R)-2-phenyl-2-(3-phenylthioureido)acetamido)-1-aza-bicyclo[4.2.0]oct-2-ene-2-carboxylic acid (5.0 g, 8.0 mmol) was added into TFA (20 mL, 80.4 mmol) at 0° C. and stirred for 24 h at 20° C. The mixture was poured into cold (0° C.) diethyl ether (50 mL) and the mixture was stirred for 30 min. The product was filtered off, washed with diethyl ether (10 ml) and dried in vacuum at 20° C. to obtain (6R,7S)-7-amino-3-chloro-8-oxo-1-aza-bicyclo[4.2.0]oct-2-ene-2-carboxylic acid (1.72 g) in 65% yield which was used for next step without further purification. ¹H NMR (400 MHz, DMSO) δ: 2.73 (s, 2H), 3.09 (d, J=10.8 Hz, 2H), 3.92 (t, J=5.6 Hz, 1H), 4.78 (d, J=5.2 Hz, 1H), 7.42 (s, 2H).

Step 3: DIPEA (0.48 mL, 2.75 mmol) was added to a suspension of (6R,7S)-7-amino-3-chloro-8-oxo-1-aza-bicyclo[4.2.0]oct-2-ene-2-carboxylic acid (746 mg, 2.75 mmol) in THF (10 mL). The suspension was stirred for 0.5 h at rt, and then diphenyl phosphorochloridate (739 mg, 2.75 mmol) was added. Sodium (E)-2-(5-chloro-2-(tritylamino)thiazol-4-yl)-2-(trityloxyimino)acetate (2.0 g, 2.75 mmol) was added with stirring at 0° C. The mixture was allowed to warm to rt, stirred for 48 h and concentrated under reduced pressure. The residue was dissolved in ethyl acetate (100 mL), washed with dilute 0.1 N HCl (15 ml), brine (15 ml), dried (Na₂SO₄), filtered and then the solvent was removed under reduced pressure to give (1.34 g) of pure product as a slight yellow solid in 54% yield which was used without further purification.

Step 4: (6R,7S,Z)-7-(2-(5-amino-1,2,4-thiadiazol-3-yl)-2-(methoxyimino)acetamido)-8-oxo-3-(trifluoromethylsulfonyloxy)-1-aza-bicyclo[4.2.0]oct-2-ene-2-carboxylic acid (8.0 g, 8.84 mmol) was dissolved in THF (150 mL) and (diazomethylene)dibenzene (8.6 g, 44.2 mmol) was added dropwise. The reaction mixture was stirred for 2 h at rt. The solvent was concentrated and the residue purified by silica gel column chromatography (hexanes/ethyl acetate, 3:1 v/v) to give (6R,7S,E)-benzhydryl 3-chloro-7-(2-(5-chloro-2-(tritylamino)thiazol-4-yl)-2-(trityloxyimino)acetamido)-8-oxo-1-aza-bicyclo[4.2.0]oct-2-ene-2-carboxylate (7.5 g) as a white solid in 80% yield.

Step 5: To a solution of (6R,7S,E)-benzhydryl-3-chloro-7-(2-(5-chloro-2-(tritylamino)thiazol-4-yl)-2-(trityloxyimino)acetamido)-8-oxo-1-aza-bicyclo[4.2.0]oct-2-ene-2-carboxylate (1.0 g, 0.9 mmol) in cold (−20° C.) DMF (35 mL), was added in one portion powdered sodium hydrogen sulfide hydrate (300 mg, 3.8 mmol). After 30 min, the reaction mixture was poured into 0.5 M monosodium phosphate (20 mL) and then extracted with ethyl acetate (100 ml). The organic layer was washed thoroughly with water (5×20 ml). After concentrating, the crude product was purified by silica gel column chromatography (eluting with hexanes/ethyl acetate, 4:1 v/v) to give pure product (720 mg) obtained in 71% yield.

### Example 20

### 5-(iodomethylthio)-1,3,4-thiadiazol-2-amine

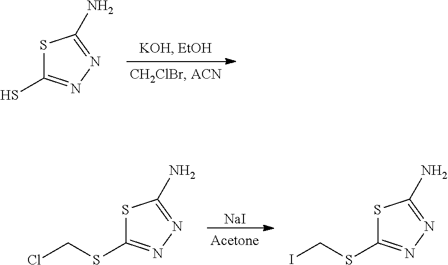

Step 1: 5-amino-1,3,4-thiadiazole-2-thiol (1.33 g, 10.0 mmol) was added to an ice-bath cooled (−5° C.) aqueous solution of 85% KOH (560 mg, 10 mmol) in EtOH (50 mL). The ice-bath was removed and the mixture was stirred until complete dissolution occurred (30 min). The solvent was then evaporated to leave a viscous oil which solidified. The resulting potassium salt was dissolved in CH₃CN (50.0 mL), and the solution was cooled to 0° C. and treated with bromochloromethane (3.88 g, 30.0 mmol). The ice in the bath was allowed to melt and the mixture was further stirred at rt for 3 h. The reaction mixture was partitioned between EtOAc (200 ml) and water (100 ml). The organic phase was dried (MgSO₄), filtered and evaporated. The residue was purified by passing through a pad of silica gel, eluted with 25% ethyl acetate/hexanes to afford 1.36 g (75%) of pure 5-(chloromethylthio)-1,3,4-thiadiazol-2-amine.

Step 2: Sodium iodide (2.94 g, 15.0 mmol) was added in one portion to a solution of 5-(chloromethylthio)-1,3,4-thiadiazol-2-amine (1.36 g, 7.5 mmol) in dry acetone (20 mL) at rt under nitrogen atmosphere which was stirred at rt for 24 h. Then the reaction mixture was filtered and concentrated under reduced pressure. The residue was partitioned between ethyl acetate (100 ml) and water (50 ml). The organic layer was evaporated and the residue was purified by the silica gel chromatography (eluting with hexanes:ethyl acetate, 1:1 v/v) to obtain pure product (1.52 g) in 76% yield.

### Example 21

### (6R,7S,Z)-3-((5-amino-1,3,4-thiadiazol-2-ylthio)methylthio)-7-(2-(2-amino-5-chlorothiazol-4-yl)-2-(hydroxyimino)acetamido)-8-oxo-1aza-bicyclo[4.2.0]oct-2-ene-2-carboxylic acid

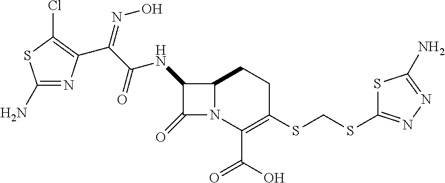

Step 1: (6R,7S,Z)-benzhydryl-3-((5-amino-1,3,4-thiadiazol-2-ylthio)methylthio)-7-(2-(5-chloro-2-(tritylamino)thiazol-4-yl)-2-(trityloxyimino)acetamido)-8-oxo-1aza-bicyclo[4.2.0]oct-2-ene-2-carboxylate was prepared from (6R,7S,Z)-benzhydryl-3-chloro-7-(2-(5-chloro-2-(tritylamino)thiazol-4-yl)-2-(trityloxyimino)acetamido)-8-oxo-1-aza-bicyclo[4.2.0]oct-2-ene-2-carboxylate (142 mg, 0.13 mmol) by following Method J. The resulting product was purified by silica column chromatography (eluting solvent: hexanes:ethyl acetate, 1:1 v/v) which resulted in a white solid (116 mg) obtained in 72% yield. LCMS ESI-MS: 1213 [M⁺]

Step 2: (6R,7S,Z)-3-((5-amino-1,3,4-thiadiazol-2-ylthio)methylthio)-7-(2-(2-amino-5-chlorothiazol-4-yl)-2-(hydroxyimino)acetamido)-8-oxo-1-aza-bicyclo[4.2.0]oct-2-ene-2-carboxylic acid was prepared from (6R,7S,Z)-benzhydryl-3-((5-amino-1,3,4-thiadiazol-2-ylthio)methylthio)-7-(2-(5-chloro-2-(tritylamino)thiazol-4-yl)-2-(trityloxyimino)acetamido)-8-oxo-1-aza-bicyclo [4.2.0]oct-2-ene-2-carboxylate (116 mg, 0.1 mmol) to give the product as a white solid (45 mg) in 83.6% yield by following Method E. LCMS ESI-MS: m/z 563 [M+H].

### Example 22

### 2-(iodomethylthio)-1,3,4-thiadiazole

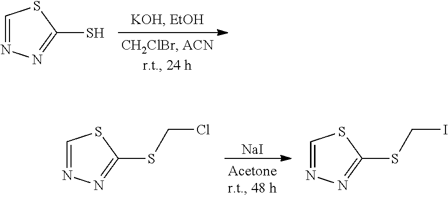

Step 1: 1,3,4-thiadiazole-2-thiol (4.0 g, 33.8 mmol, 1 eq) was added to an ice-bath cooled (−5° C.) aqueous solution of 85% KOH (1.9 g, 33.8 mmol, 1 eq) in ethanol (120 mL). The ice-bath was removed and the mixture was stirred until complete dissolution occurred. The solvent was then evaporated to leave viscous oil which solidified upon standing in a few h at rt. The resulting potassium salt was dissolved in CH₃CN (120 mL), and the solution was cooled in an ice-bath and treated with bromochloromethane (13.2 g, 101.5 mmol, 3 eq). The ice in the bath was allowed to melt and the mixture was further stirred at rt for 24 h. The reaction mixture was partitioned between ethyl acetate (200 ml) and water (50 ml). The organic phase was dried with MgSO₄, filtered, and evaporated to give the product (4.2 g) in 75% yield. LCMS ESI-MS: m/z 166.9 [M+H].

Step 2: Sodium iodide (7.2 g, 48 mmol, 2 eq) was added in one portion to a solution of 2-(chloromethylthio)-1,3,4-thiadiazole (4.0 g, 24 mmol, 1 eq) in dry acetone (200 mL) at rt under nitrogen. The heterogeneous reaction was stirred at rt for 48 h, then filtered and concentrated to dryness under reduced pressure. The residue was partitioned between ethyl acetate (200 ml) and water (50 ml). The organic layer was evaporated and the residue purified by silica gel chromatography (hexanes:ethyl acetate 1:1 v/v). The pure product (4.95 g) was obtained in 80% yield. LCMS, ESI-MS: m/z 258.8 [M+H].

### Example 23

### (6R,7S,Z)-3-((1,3,4-thiadiazol-2-ylthio)methylthio)-7-(2-(2-amino-5-chlorothiazol-4-yl)-2-(hydroxyimino)acetamido)-8-oxo-1-aza-bicyclo[4.2.0]oct-2-ene-2-carboxylic acid

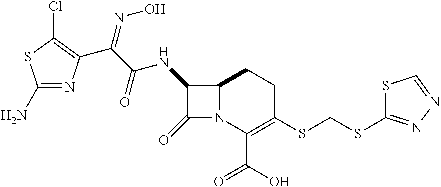

Step 1: (6R,7S,Z)-benzhydryl-3-((1,3,4-thiadiazol-2-ylthio)methylthio)-7-(2-(5-chloro-2-(tritylamino)thiazol-4-yl)-2-(trityloxyimino)acetamido)-8-oxo-1aza-bicyclo[4.2.0]oct-2-ene-2-carboxylate was prepared from (6R,7S,Z)-benzhydryl-7-(2-(5-chloro-2-(tritylamino)thiazol-4-yl)-2-(trityloxyimino)acetamido)-3-mercapto-8-oxo-1-aza-bicyclo[4.2.0]oct-2-ene-2-carboxylate (110 mg, 0.1 mmol) by following Method J. The resulting crude product was purified by silica gel column chromatography (eluting solvent hexanes:ethyl acetate, 2:1 v/v) to give the pure product as a white solid (58 mg) in 47% yield. LCMS, ESI-MS: m/z 1220 [M+Na].

Step 2: (6R,7S,Z)-3-((1,3,4-thiadiazol-2-ylthio)methylthio)-7-(2-(2-amino-5-chlorothiazol-4-yl)-2-(hydroxyimino)acetamido)-8-oxo-1-aza-bicyclo[4.2.0]oct-2-ene-2-carboxylic acid was prepared from (6R,7S,Z)-benzhydryl-3-((1,3,4-thiadiazol-2-ylthio)methylthio)-7-(2-(5-chloro-2-(tritylamino)thiazol-4-yl)-2-(trityloxyimino)acetamido)-8-oxo-1aza-bicyclo[4.2.0]oct-2-ene-2-carboxylate (58 mg, 0.05 mmol) by following Method E. The resulting product was washed with diethyl ether (5 mL) and dried in vacuo to obtain the pure target product (16 mg) in 60% yield. LCMS, ESI-MS: m/z 547.7 [M+H].

### Example 24

### 5-(iodomethylthio)-N-methyl-1,3,4-thiadiazol-2-amine

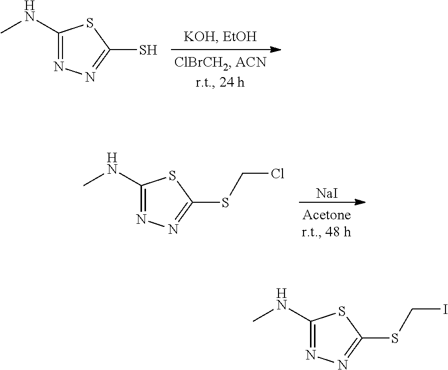

Step 1: 5-(methylamino)-1,3,4-thiadiazole-2-thiol (5.0 g, 33.8 mmol, 1 eq) was added to an ice-bath cooled (−5° C.) aqueous solution of 85% KOH (1.9 g, 33.8 mmol, 1 eq) in ethanol (120 mL). The ice-bath was removed and the mixture stirred until complete dissolution occurred. The solvent was then evaporated to leave a viscous oil which solidified upon standing after a few h. The resulting potassium salt was dissolved in CH₃CN (120 mL), and the solution was cooled in an ice-bath and treated with bromochloromethane (13.2 g, 101.5 mmol, 3 eq) with stirring. The ice in the bath was allowed to melt and the mixture was further stirred at rt for 24 h. The reaction mixture was partitioned between ethyl acetate (200 mL) and water (50 mL). The organic phase was dried with MgSO₄, filtered and evaporated to get the product (4.9 g) as a white solid in 74% yield. LCMS, ESI-MS: m/z 195.9 [M+H].

Step 2: Sodium iodide (7.2 g, 48 mmol, 2 eq) was added in one portion to a solution of 5-(chloromethylthio)-N-methyl-1,3,4-thiadiazol-2-amine (4.7 g, 24 mmol, 1 eq) in dry acetone (200 mL) at rt with stirring under nitrogen. The heterogeneous reaction was stirred at rt for 48 h, then filtered and concentrated under reduced pressure to dryness. The residue was partitioned between ethyl acetate (200 mL) and water (950 mL). The organic layer was evaporated to dryness and the residue purified by silica gel chromatography (eluting solvent: hexanes:ethyl acetate, 1:1 v/v). The pure product (5.5 g) was obtained as a white solid in 80% yield. LCMS, ESI-MS: m/z 287.8 [M+H].

### Example 25

### (6R,7S,Z)-7-(2-(2-amino-5-chlorothiazol-4-yl)-2-(hydroxyimino)acetamido)-3-((5-(methylamino)-1,3,4-thiadiazol-2-ylthio)methylthio)-8-oxo-1-aza-bicyclo[4.2.0]oct-2-ene-2-carboxylic acid

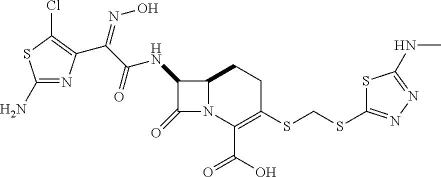

Step 1: (6R,7S,Z)-benzhydryl-7-(2-(5-chloro-2-(tritylamino)thiazol-4-yl)-2-(trityloxyimino)acetamido)-3-((5-(methylamino)-1,3,4-thiadiazol-2-ylthio)methylthio)-8-oxo-1aza-bicyclo[4.2.0]oct-2-ene-2-carboxylate was prepared from (6R,7S,Z)-benzhydryl-7-(2-(5-chloro-2-(tritylamino)thiazol-4-yl)-2-(trityloxyimino)acetamido)-3-mercapto-8-oxo-1-aza-bicyclo[4.2.0]oct-2-ene-2-carboxylate (109 mg, 0.1 mmol) by following Method J. The resulting crude product was purified by silica gel column chromatography (eluting solvent: hexanes:ethyl acetate, 2:1 v/v) to give the pure product (52 mg) as a slight yellow solid in 45% yield. LCMS, ESI-MS: m/z 287.8 [M+H].

Step 2: (6R,7S,Z)-7-(2-(2-amino-5-chlorothiazol-4-yl)-2-(hydroxyimino)acetamido)-3-((5-(methylamino)-1,3,4-thiadiazol-2-ylthio)methylthio)-8-oxo-1-aza-bicyclo[4.2.0]oct-2-ene-2-carboxylic acid was prepared from (6R,7S,Z)-benzhydryl-7-(2-(5-chloro-2-(tritylamino)thiazol-4-yl)-2-(trityloxyimino)acetamido)-3-((5-(methylamino)-1,3,4-thiadiazol-2-ylthio)methylthio)-8-oxo-1aza-bicyclo[4.2.0]oct-2-ene-2-carboxylate (52 mg, 0.05 mmol) by following Method E. The resulting product was washed with diethyl ether (5 ml) and dried in vacuo to give (25 mg) as a slightly yellow solid in 94% yield. LCMS, ESI-MS: m/z 576.7 [M+H].

### Example 26

### tert-Butyl 4-(bromomethyl)-1H-pyrazole-1-carboxylate

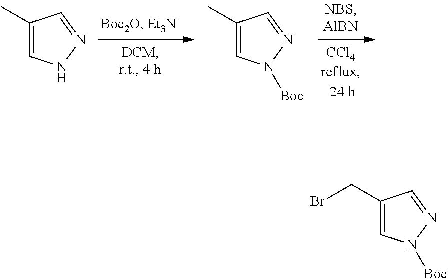

Step 1: Et₃N (3 mL, 20 mmol, 2 eq) and then Boc₂O (2.4 g, 11 mmol, 1.1 eq) were added to a solution of 4-methyl-1H-pyrazole (820 mg, 10 mmol, 1 eq) dissolved in DCM (10 mL) at rt. The mixture was stirred at rt for 4 h. After that, the reaction mixture was washed with water (10 mL), the aqueous layer was extracted with DCM (3×20 mL), dried over Na₂SO₄, filtered and concentrated under reduced pressure to give the product (1.53 g) in 70% yield as a white solid. LCMS, ESI-MS: m/z 127.1 [M+H−56].

Step 2: To a solution of 4-methylpyrazole-1-carboxylic acid tert-butyl ester (2.0 g, 11 mmol, 1 eq) in carbon tetrachloride was added N-bromosuccinimide (2.0 g, 12 mmol, 1.1 eq) and AIBN (0.36 g, 2.2 mmol, 0.2 eq) at rt with stirring. The reaction mixture was heated at reflux for 24 h. The reaction mixture was cooled to rt, filtered, and concentrated under reduced pressure. The residue was purified by silica gel column chromatography (eluting solvent: hexanes:ethyl acetate, 4:1 v/v) to obtain the pure target product (0.9 g) as a white solid in 30% yield. LCMS, ESI-MS: m/z 205 [M+H−56].

### Example 27

### (6R,7S,Z)-3-((1H-pyrazol-4-yl)methylthio)-7-(2-(2-amino-5-chlorothiazol-4-yl)-2-(hydroxyimino)acetamido)-8-oxo-1-aza-bicyclo[4.2.0]oct-2-ene-2-carboxylic acid

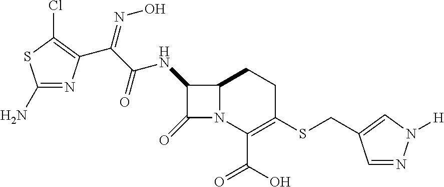

Step 1: (6R,7S,Z)-benzhydryl-3-((1-(tert-butoxycarbonyl)-1H-pyrazol-4-yl)methylthio)-7-(2-(5-chloro-2-(tritylamino)thiazol-4-yl)-2-(trityloxyimino)acetamido)-8-oxo-1aza-bicyclo[4.2.0]oct-2-ene-2-carboxylate was prepared from (6R,7S,Z)-benzhydryl-7-(2-(5-chloro-2-(tritylamino)thiazol-4-yl)-2-(trityloxyimino)acetamido)-3-mercapto-8-oxo-1-aza-bicyclo[4.2.0]oct-2-ene-2-carboxylate (194 mg, 0.18 mmol) by following Method J. The resulting crude product was purified by silica gel column chromatography (eluting solvent: hexanes:ethyl acetate, 2:1 v/v) to give the pure product as a slight yellow solid (107 mg) in 47% yield. LCMS, ESI-MS: m/z 1248 [M+H].

Step 2: (6R,7S,Z)-3-((1H-pyrazol-4-yl)methylthio)-7-(2-(2-amino-5-chlorothiazol-4-yl)-2-(hydroxyimino)acetamido)-8-oxo-1-aza-bicyclo[4.2.0]oct-2-ene-2-carboxylic acid was prepared from 3-((1-(tert-butoxycarbonyl)-1H-pyrazol-4-yl)methylthio)-7-(2-(5-chloro-2-(tritylamino)thiazol-4-yl)-2-(trityloxyimino)acetamido)-8-oxo-1-aza-bicyclo[4.2.0]oct-2-ene-2-carboxylate (107 mg, 0.08 mmol) by following Method E. The resulting crude product was purified by silica gel column chromatography (eluting solvent hexanes:ethyl acetate, 2:1 v/v) to give the pure product as a slightly yellow solid (20 mg) in 47% yield. The resulting pure product was then washed with diethyl ether (5 ml) to give a finely divided slight yellow solid (15 mg) in 38% yield. LCMS, ESI-MS: m/z 497.8 [M+H].

### Example 28

### ((4-Methylthiazol-5-yl)methyl)triphenylphosphonium bromide

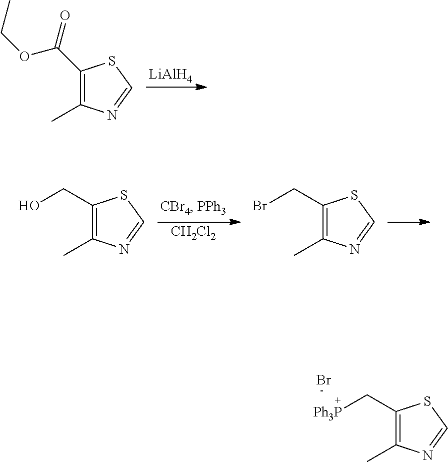

Step 1: To a stirred mixture of lithium aluminum hydride (3.8 g, 100 mmol) in THF (100 mL) at 5-10° C. was added ethyl 4-methylthiazole-5-carboxylate (10 g, 58 mmol) over a period of 30 minutes with stirring under nitrogen. The reaction mixture was stirred at 10-15° C. for 2 to 3 h. Progress of reaction was monitored by TLC (ethyl acetate:hexanes=1:1). After reaching completion, the reaction was quenched by adding an aqueous solution of saturated sodium sulfate (20 mL). The resultant inorganic solids were filtered and the filter cake was washed with ethyl acetate (3×10 mL). Filtrate and organic washes were concentrated under reduced pressure to give a pale yellow solid (7.5 g) in high purity by HPLC and in 100% yield. LCMS, ESI-MS: m/z 130 [M+H], 1H-NMR (300 MHz): 2.36 (s, 3H), 3.98 (s broad, 1H), 4.79 (s, 2H), 8.58 (s, 1H).

Step 2: To a 250-mL, round-bottomed flask fitted with a septum cap were added (4-methylthiazol-5-yl)methanol (2.6 g, 20.0 mmol), CBr₄ (8 g, 24 mmol) and CH₂Cl₂ (100 mL) at rt under nitrogen with stirring. The solution was cooled with an ice-water bath. After cooling, Ph₃P (7.4 g, 28 mmol) in CH₂Cl₂ (20 mL) was added drop wise via syringe. After addition was complete, the ice bath was removed and the mixture stirred for an additional 6 h at rt. The solvent was removed under reduced pressure and the residue extracted into ether (5×40 mL). The ether layer was concentrated under reduced pressure and the residue was purified by silica gel column chromatography (ethyl acetate:hexanes, 1:30 v/v) to give the pure product as a white solid (3 g) in 40% yield. LCMS, ESI-MS: m/z 192 [M+H].

Step 3: To a stirred mixture of 5-(bromomethyl)-4-methylthiazole (191 mg, 1 mmol) in DCM (10 mL) at rt was added PPh₃ (262 mg) in DCM (10 mL). The reaction mixture was heated and stirred at 40° C. for 2 to 3 h. Progress of reaction was monitored by TLC (CH₃OH:DCM=1:10). After coming to completion, the reaction was concentrated under reduced pressure and the residue purified by silica gel column chromatography (CH₃OH:DCM=1:20) to give the pure product as a white solid (22 mg) in 50% yield. LCMS, ESI-MS: m/z 374 [M−Br].

### Example 29

### (6R,7S)-7-((Z)-2-(2-amino-5-chlorothiazol-4-yl)-2-(hydroxyimino)acetamido)-3-((Z)-2-(4-methylthiazol-5-yl)vinyl)-8-oxo-1-aza-bicyclo[4.2.0]oct-2-ene-2-carboxylic acid (Isomer Mixture, E:Z 2:5)

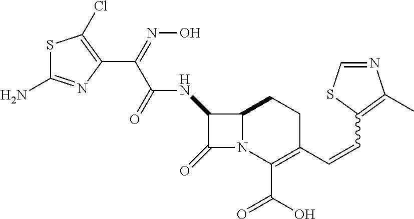

Step 1: (6R,7S,Z)-7-amino-3-(2-(4-methylthiazol-5-yl)vinyl)-8-oxo-1-aza-bicyclo[4.2.0]oct-2-ene-2-carboxylic acid (isomer mixture, E:Z=2:5) was prepared from (6R,7S,Z)-tert-butyl 7-(tert-butoxycarbonylamino)-3-(2-(4-methylthiazol-5-yl)vinyl)-8-oxo-1-aza-bicyclo[4.2.0]oct-2-ene-2-carboxylate (isomer mixture, E:Z=2:5) (112 mg, 0.37 mmol) by following Method A. The resulting product was isolated as a white powder (153 mg) in 90% yield and was used without further purification. LCMS, ESI-MS: m/z 306 [M+H].

Step 2: (6R,7S)-7-((Z)-2-(5-chloro-2-(tritylamino)thiazol-4-yl)-2-(trityloxyimino)acetamido)-3-((Z)-2-(4-methylthiazol-5-yl)vinyl)-8-oxo-1-aza-bicyclo[4.2.0]oct-2-ene-2-carboxylic acid (isomer mixture, E:Z=2:5) was prepared from (6R,7S,Z)-7-amino-3-(2-(4-methylthiazol-5-yl)vinyl)-8-oxo-1-aza-bicyclo[4.2.0]oct-2-ene-2-carboxylic acid (isomer mixture, E:Z=2:5) (153 mg, 0.33 mmol) by following Method B. The resulting product was isolated as a white powder (316 mg) in 96% yield and was used without further purification. LCMS, ESI-MS: m/z 993 [M+H].

Step 3: (6R,7S)-benzhydryl 7-((Z)-2-(5-chloro-2-(tritylamino)thiazol-4-yl)-2-(trityloxyimino)acetamido)-3-((Z)-2-(4-methylthiazol-5-yl)vinyl)-8-oxo-1-aza-bicyclo[4.2.0]oct-2-ene-2-carboxylate (isomer mixture, E:Z=2:5) was prepared from (6R,7S)-7-((Z)-2-(5-chloro-2-(tritylamino)thiazol-4-yl)-2-(trityloxyimino)acetamido)-3-((Z)-2-(4-methylthiazol-5-yl)vinyl)-8-oxo-1-aza-bicyclo[4.2.0]oct-2-ene-2-carboxylic acid (isomer mixture, E:Z=2:5) (316 mg, 0.32 mmol) by following Method C. The resulting mixture was purified by silica gel column chromatography (eluting solvent hexanes:ethyl acetate, 3:2 v/v) to give the pure produce (147 mg) as a slight yellow solid in 40% yield. LCMS, ESI-MS: m/z 1181 [M+Na].

Step 4: (6R,7S)-7-((Z)-2-(2-amino-5-chlorothiazol-4-yl)-2-(hydroxyimino)acetamido)-3-((Z)-2-(4-methylthiazol-5-yl)vinyl)-8-oxo-1-aza-bicyclo[4.2.0]oct-2-ene-2-carboxylic acid (isomer mixture, E:Z=2:5) was prepared from (6R,7S)-benzhydryl-7-((Z-2-(5-chloro-2-(tritylamino)thiazol-4-yl)-2-(trityloxyimino)acetamido)-3-((Z)-2-(4-methylthiazol-5-yl)vinyl)-8-oxo-1-aza-bicyclo[4.2.0]oct-2-ene-2-carboxylate (isomer mixture, E:Z=2:5) (147 mg, 0.13 mmol) by following Method E. The crude product was triturated with ethyl ether (5 mL) to give the product as a finely divided slight yellow solid (22 mg) in 34% yield. LCMS, ESI-MS: 509.0 [M+H].

### Example 30

### (6R,7S,Z)-tert-butyl 7-(tert-butoxycarbonylamino)-3-(2-(4-methylthiazol-5-yl)vinyl)-8-oxo-1aza-bicyclo[4.2.0]oct-2-ene-2-carboxylate

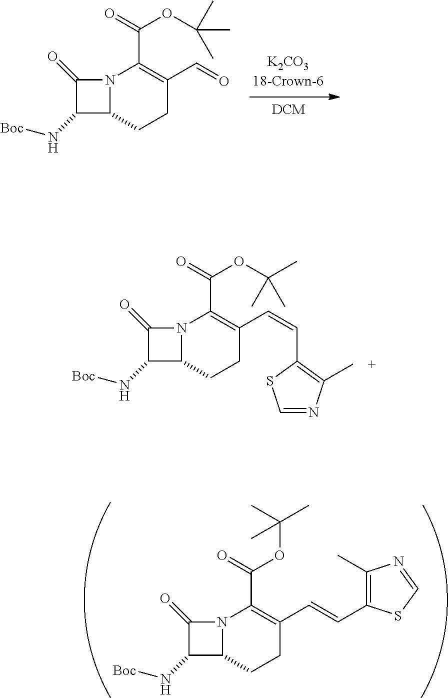

Step 1: K₂CO₃ (90 mg, 0.65 mmol) and 18-crown-6 (6 mg, 23 mmol) were added to a stirred solution of (6R,7S)-tert-butyl 7-(tert-butoxycarbonylamino)-3-formyl-8-oxo-1aza-bicyclo[4.2.0]oct-2-ene-2-carboxylate (200 mg, 546 mmol) and triphenylpyridin-4-ylmethylphosphonium bromide (300 mg, 600 mmol) in anhydrous DCM (30 mL) at rt under nitrogen. After 3 h, more K₂CO₃ (23 mg, 0.17 mmol) was added and stirring was continued for an additional 3 h. The mixture was partitioned between DCM (25 mL) and H₂O (10 mL). The organic phase was washed with H₂O (10 mL), dried (MgSO₄), filtrated and concentrated under reduced pressure. The residue was purified by reverse phase HPLC (eluting from 5% to 95% acetonitrile in water using 0.1% TFA on a C18 column (25 mm×150 mm)). The product fractions were collected and lyophilized to give (6R,7S,Z)-tert-butyl 7-(tert-butoxycarbonylamino)-3-(2-(4-methylthiazol-5-yl)vinyl)-8-oxo-1-aza-bicyclo[4.2.0]oct-2-ene-2-carboxylate (pure Z isomer) (90 mg) as a white solid in 45% yield (¹H-NMR (400 MHz, CDCl₃): 1.42 (s, 9H), 1.45 (s, 9H), 2.04 (s, 1H), 2.35 (d, J=4.2 Hz 2H), 2.45 (s, 3H), 3.85-3.87 (m, 1H), 5.21-5.25 (m, 1H), 5.47 (d, J=5.5 Hz 1H), 6.35 (d, J=12 Hz 1H), 6.57 (d, J=12 Hz, 1H), 8.61 (s, 1H)) and (6R,7S,E)-tert-butyl 7-(tert-butoxycarbonylamino)-3-(2-(4-methylthiazol-5-yl)vinyl)-8-oxo-1-aza-bicyclo[4.2.0]oct-2-ene-2-carboxylate (pure E isomer) (45 mg) as a white solid in 27% yield (LCMS, ESI-MS: m/z 461 [M+H], ¹H-NMR (400 MHz, CDCl₃): 1.46 (s, 9H), 1.59 (s, 9H), 2.16-2.19 (m, 1H), 2.38-2.45 (m, 1H), 2.46 (s, 3H), 2.81-2.89 (m, 1H), 3.80-3.88 (m, 1H), 5.21 (d, J=4 Hz, 2H), 6.80 (d, J=16 Hz 1H), 7.44 (d, J=16 Hz 1H), 8.58 (s, 1H))

### Example 31

### (6R,7S)-7-((Z)-2-(2-amino-5-chlorothiazol-4-yl)-2-(hydroxyimino)acetamido)-3-((Z)-2-(4-methylthiazol-5-yl)vinyl)-8-oxo-1-aza-bicyclo[4.2.0]oct-2-ene-2-carboxylic acid

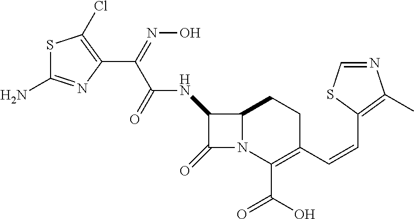

Step 1: (6R,7S,Z)-7-amino-3-(2-(4-methylthiazol-5-yl)vinyl)-8-oxo-1-aza-bicyclo[4.2.0]oct-2-ene-2-carboxylic acid (pure Z isomer) was prepared from (6R,7S,Z)-tert-butyl-7-(tert-butoxycarbonylamino)-3-(2-(4-methylthiazol-5-yl)vinyl)-8-oxo-1-aza-bicyclo[4.2.0]oct-2-ene-2-carboxylate (840 mg, 1.82 mmol) by following Method A. The resulting product (500 mg) was isolated as a white powder in 90% yield and used without further purification. LCMS, ESI-MS: m/z 306 [M+H].

Step 2: (6R,7S)-7-((Z)-2-(5-chloro-2-(tritylamino)thiazol-4-yl)-2-(trityloxyimino)acetamido)-3-((Z)-2-(4-methylthiazol-5-yl)vinyl)-8-oxo-1-aza-bicyclo[4.2.0]oct-2-ene-2-carboxylic acid was prepared from (6R,7S,Z)-7-amino-3-(2-(4-methylthiazol-5-yl)vinyl)-8-oxo-1-aza-bicyclo[4.2.0]oct-2-ene-2-carboxylic acid (288 mg, 0.94 mmol) by following Method B. The resulting product (900 mg) was isolated as a white powder in 96% yield and used without further purification. LCMS, ESI-MS: m/z 993 [M+H].

Step 3: (6R,7S)-benzhydryl 7-((Z)-2-(5-chloro-2-(tritylamino)thiazol-4-yl)-2-(trityloxyimino)acetamido)-3-((Z)-2-(4-methylthiazol-5-yl)vinyl)-8-oxo-1-aza-bicyclo[4.2.0]oct-2-ene-2-carboxylate was prepared from (6R,7S)-7-((Z)-2-(5-chloro-2-(tritylamino)thiazol-4-yl)-2-(trityloxyimino)acetamido)-3-((Z)-2-(4-methylthiazol-5-yl)vinyl)-8-oxo-1-aza-bicyclo[4.2.0]oct-2-ene-2-carboxylic acid (646 mg, 0.65 mmol) by following Method C. The resulting crude reaction was purified by silica gel column chromatography (eluting solvent hexanes:ethyl acetate, 3:2 v/v) to give pure product (300 mg) as a slightly yellow solid in 40% yield. LCMS, ESI-MS: m/z 1181 [M+Na].

Step 4: (6R,7S)-7-((Z)-2-(2-amino-5-chlorothiazol-4-yl)-2-(hydroxyimino)acetamido)-3-((Z)-2-(4-methylthiazol-5-yl)vinyl)-8-oxo-1-aza-bicyclo[4.2.0]oct-2-ene-2-carboxylic acid was prepared from (6R,7S)-benzhydryl 7-((Z)-2-(5-chloro-2-(tritylamino)thiazol-4-yl)-2-(trityloxyimino)acetamido)-3-((Z)-2-(4-methylthiazol-5-yl)vinyl)-8-oxo-1-aza-bicyclo[4.2.0]oct-2-ene-2-carboxylate (168 mg, 0.15 mmol) by following Method E. After triturating with ethyl ether (5 ml), the pure final product (25 mg) was obtained as a slightly yellow solid in 33.8% yield. LCMS, ESI-MS: 509.0 [M+H], ¹H-NMR (400 MHz, DMSO-d6): 1.64-1.69 (m, 1H), 1.88 (d, J=12.8 Hz, 1H), 2.20-2.25 (m, 2H), 2.36 (s, 3H), 3.86-3.92 (m, 1H), 5.49 (dd, J=4.2 Hz; J=3.6 Hz, 1H), 6.36 (d, J=12 Hz, 1H), 6.63 (d, J=12 Hz, 1H), 7.31 (s, 2H), 8.92 (s, 1H), 9.21 (d, J=9 Hz 1H), 11.69 (s, 1H), 12.92 (s, 1H).

### Example 32

### ((3′R)-1′-(allyloxycarbonyl)-2-oxo-1,3′-bipyrrolidin-3-yl)triphenylphosphonium bromide

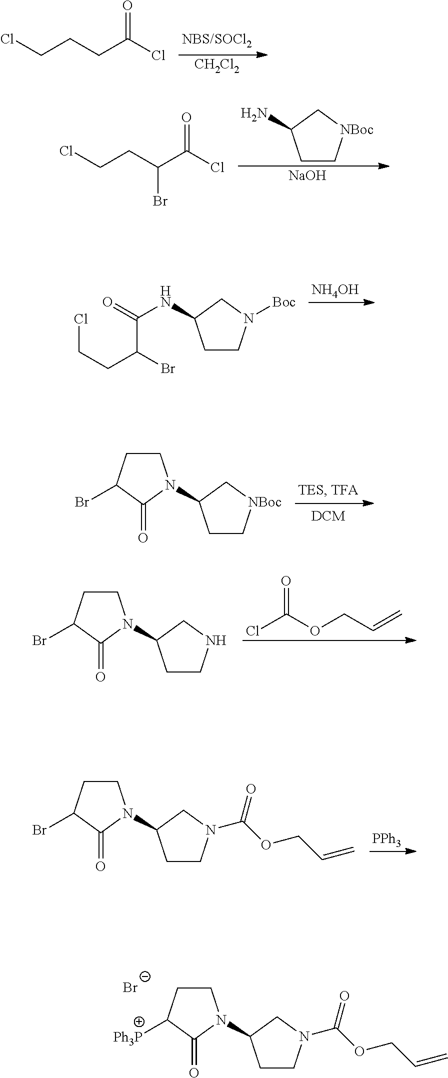

Step 1: To a suspension of 4-chlorobutanoyl chloride (140 g, 1.0 mol, 1.0 equiv) and NBS (270 g, 1.5 mol, 1.5 equiv) in CH₂Cl₂ (250 mL) was added. SOCl₂ (6 g, 0.05 mol, 0.01 equiv) and then 40% HBr (5 mL) was added dropwise. After refluxing for 1.5 h, hexane (500 mL) was added. The suspension was filtered and the filtrate was concentrate under reduced pressure to give the pure product (118 g) in yield 80%.

Step 2: To a mixture of (R)-tert-butyl 3-aminopyrrolidine-1-carboxylate (372 g, 0.24 mol, 1.1 equiv) and 50% aqueous NaOH (80 mL) in CH₂Cl₂ (25 mL) was added 2-bromo-4-chlorobutanoyl chloride (48 g, 0.22 mol, 1.0 equiv) in CH₂Cl₂ (250 mL) solution at rt with stirring. After 3 h, 10% aqueous (n-Bu)₄NOH (11.4 mL) was added at rt and stirred overnight. The mixture was washed with water (50 ml) and extracted with CH₂Cl₂ (2×100 mL). The organic layer was separated, dried over Na₂SO₄, filtered and the filtrate was concentrated under reduced pressure to give the product (58.4 g) as an off white solid in 80% yield. LCMS, ESI-MS: 333 [M+H].

Step 3: To a solution of (3′R)-tert-butyl-3-bromo-2-oxo-1,3′-bipyrrolidine-1′-carboxylate (3 g, 9 mmol) in DCM (6 mL) was added triethylsilane (3 mL, 18.9 mmol). TFA (6 mL) was added to the precooked 0° C. mixture, and then allowed to warm to rt. After stirring for 4 h, the reaction was concentrated under reduced pressure and triturated with petroleum ether to obtain the product (2.7 g) as yellow oil in 87%. LCMS, ESI-MS: 233 [M+H].

Step 4: To a solution of (3R)-3-bromo-1,3′-bipyrrolidin-2-one (500 mg, 2.1 mmol) in THF (6 mL) and H₂O (2 mL) was added potassium carbonate (445 mg, 3.2 mmol) and allyl chloroformate (517 mg, 4.3 mmol). After stirring at rt for 2 h, the mixture was extracted with DCM (10 mL), the organic phase was washed with water (5 mL), dried (Na₂SO₄), filtered and concentrated under reduce pressure to obtain the pure product (270 mg) as a yellow oil in 40% yield. The resulting product was used without further purification. LCMS, ESI-MS: 317 [M+H].

Step 5: Triphenylphosphine (1 g, 3.8 mmol, 1.0 equiv) and (3′R)-allyl 3-bromo-2-oxo-1,3′-bipyrrolidine-1′-carboxylate (1.2 g, 3.8 mmol, 1.0 equiv) were dissolved in DCM (20 mL). The solvent was removed under reduced pressure and the residual oil was heated for 2 h with stirring at 100° C. The resulting solid was dissolved in DCM (10 mL). n-Hexane (100 mL) was added to the reaction and stirred. A fine solid was formed and collected by filtration. The crude product was purified by silica gel column chromatography (eluting solvent: CH₃OH:DCM, 1:10 v/v) to afford desired compound (1.33 g) as a white solid in 61% yield. LCMS, ESI-MS: 499 [M−Br].

### Example 33

### (6R,7S)-3-((Z)-((R)-1′-(allyloxycarbonyl)-2-oxo-1,3′-bipyrrolidin-3-ylidene)methyl)-7-amino-8-oxo-1-aza-bicyclo[4.2.0]oct-2-ene-2-carboxylic acid

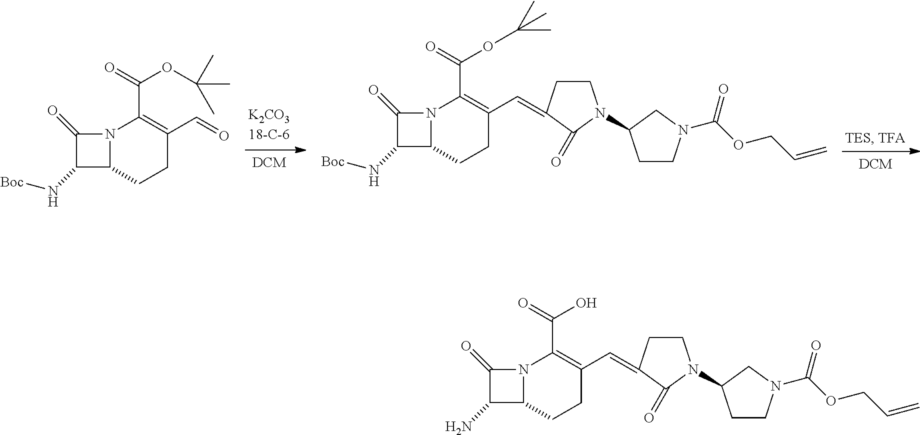

Step 1: K₂CO₃ (135 mg, 0.98 mmol) and 18-crown-6 (6 mg, 0.23 mmol) were added to a stirred solution of ((3′R)-1′-(allyloxycarbonyl)-2-oxo-1,3′-bipyrrolidin-3-yl)triphenylphosphonium bromide (569 mg, 098 mmol) in DCM (10 mL). After stirring for 0.5 h at rt, (6R,7S)-tert-butyl 7-(tert-butoxycarbonylamino)-3-formyl-8-oxo-1-aza-bicyclo[4.2.0]oct-2-ene-2-carboxylate (300 mg, 0.82 mmol) was added. After stirring for an additional 5 h, the mixture was partitioned between DCM (25 mL) and H₂O (10 mL). The organic phase was washed with H₂O (10 mL), dried (MgSO₄), filtrated and concentrated under reduced pressure. The residue was purified by silica gel column chromatography (eluting solvent: hexanes:ethyl acetate, 1:5 v/v) to afford the pure product (110 mg) ((6R,7S)-tert-butyl 3-((Z)-((R)-1′-(allyloxycarbonyl)-2-oxo-1,3′-bipyrrolidin-3-ylidene)methyl)-7-(tert-butoxycarbonylamino)-8-oxo-1aza-bicyclo[4.2.0]oct-2-ene-2-carboxylate as a white solid in 23% yield. LCMS, ESI-MS: 587 [M+H].

Step 2: To a solution of (6R,7S)-tert-butyl-3-((Z)-((R)-1′-(allyloxycarbonyl)-2-oxo-1,3′-bipyrrolidin-3-ylidene)methyl)-7-(tert-butoxycarbonylamino)-8-oxo-1-aza-bicyclo[4.2.0]oct-2-ene-2-carboxylate (190 mg, 0.32 mmol) in DCM (1.5 mL) was added triethylsilane (0.5 mL, 3.15 mmol). The mixture was cooled down to 0° C. and TFA (1 mL) was added. After warming to rt and stirring for 2 h, the reaction was concentrated under reduced pressure, and the residue triturated with petroleum ether to obtain the product (150 mg) as a yellow solid in 85% yield. LCMS, ESI-MS: 431 [M+H].

### Example 34

### (6R,7S)-7-((Z)-2-(2-amino-5-chlorothiazol-4-yl)-2-(hydroxyimino)acetamido)-8-oxo-3-((E)-((R)-2-oxo-1,3′-bipyrrolidin-3-ylidene)methyl)-1-aza-bicyclo[4.2.0]oct-2-ene-2-carboxylic acid

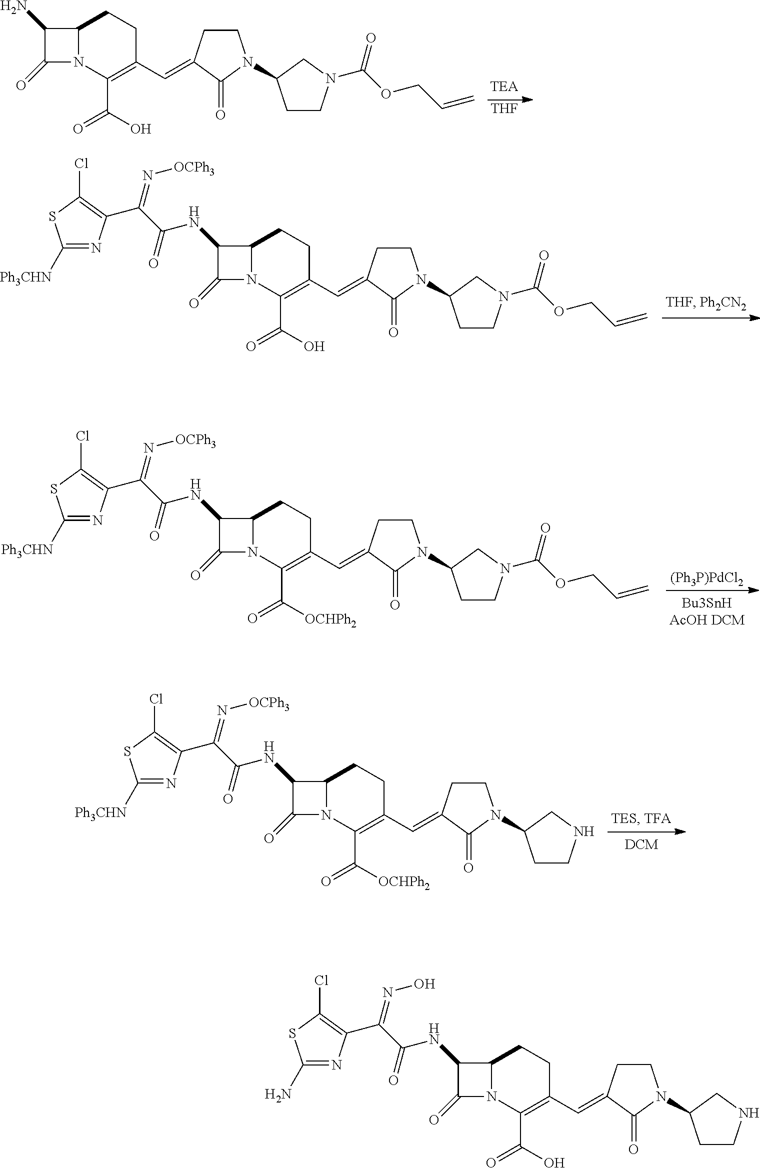

Step 1: Triethylamine (136 mg, 1.35 mmol) was added to (6R,7S)-3-((E)-((R)-1′-(allyloxycarbonyl)-2-oxo-1,3′-bipyrrolidin-3-ylidene)methyl)-7-amino-8-oxo-1-aza-bicyclo[4.2.0]oct-2-ene-2-carboxylic acid (150 mg, 0.27 mmol) suspended in THF (15 mL). The suspension was stirred for 0.5 h at rt, and (Z)-S-benzo[d]thiazol-2-yl-2-(5-chloro-2-(tritylamino)thiazol-4-yl)-2-(trityloxyimino)ethanethioate (370 g, 0.3 mmol) was added with stirring at 0° C. The mixture was allowed to warm to rt. After stirring for 18 h, the reaction was concentrated under reduced pressure. The residue was dissolved in ethyl acetate (25 ml), washed with dilute 1 M HCl solution (5 mL), brine (5 mL), dried (Na₂SO₄), filtered and concentrated under reduced pressure. The resulting product (450 mg) as a slightly yellow solid was used without further purification. LCMS, ESI-MS: 1118 [M+H].

Step 2: (6R,7S)-3-((E)-((R)-1′-(allyloxycarbonyl)-2-oxo-1,3′-bipyrrolidin-3-ylidene)methyl)-7-((Z)-2-(5-chloro-2-(tritylamino)thiazol-4-yl)-2-(trityloxyimino)acetamido)-8-oxo-1-aza-bicyclo[4.2.0]oct-2-ene-2-carboxylic acid (450 mg, crude solid) was dissolved in THF (15 mL) and (diazomethylene)dibenzene (390 mg, 2 mmol) was added dropwise. The reaction mixture was stirred for 2 h at rt. The solvent was then concentrated under reduced pressure and purified by silica gel column chromatography (eluting solvent hexanes:ethyl acetate, 1:10 v/v) to obtain the pure product (102 mg) as a yellow solid in 23% yield over the 2 steps. LCMS, ESI-MS: 1284 [M+H].

Step 3: To a solution of (6R,7S)-benzhydryl-3-((E)-((R)-1′-(allyloxycarbonyl)-2-oxo-1,3′-bipyrrolidin-3-ylidene)methyl)-7-((Z)-2-(5-chloro-2-(tritylamino)thiazol-4-yl)-2-(trityloxyimino)acetamido)-8-oxo-1-aza-bicyclo[4.2.0]oct-2-ene-2-carboxylate (102 mg, 0.08 mmol) in DCM (2 mL) was added Pd(PPh₃)₂Cl₂ (8 mg, 0.02 mmol) and gal. acetic acid (1 drop) with stirring at rt under nitrogen. To the resulting mixture was added Bu₃SnH (40 mg, 0.09 mmol). After 1 h, the mixture was concentrated under reduce pressure. The residue was triturated with Et₂O to give (6R,7S)-benzhydryl-7-((Z)-2-(5-chloro-2-(tritylamino)thiazol-4-yl)-2-(trityloxyimino)acetamido)-8-oxo-3-((E)-((R)-2-oxo-1,3′-bipyrrolidin-3-ylidene)methyl)-1-aza-bicyclo[4.2.0]oct-2-ene-2-carboxylate (90 mg) as a white solid in 94% yield. LCMS, ESI-MS: 1200 [M+H].

Step 4: To a solution of (6R,7S)-benzhydryl-7-((Z)-2-(5-chloro-2-(tritylamino)thiazol-4-yl)-2-(trityloxyimino)acetamido)-8-oxo-3-((E)-((R)-2-oxo-1,3′-bipyrrolidin-3-ylidene)methyl)-1-aza-bicyclo[4.2.0]oct-2-ene-2-carboxylate (90 mg, 0.075 mmol) in DCM (1 mL) was added triethylsilane (0.5 mL, 3.15 mmol) with stirring at rt. The mixture was cooled to 0° C. and TFA (1 mL) was added. After warming to rt and stirring for 2 h, the reaction was concentrated under reduced pressure. The residue was triturated with Et₂O (5 mL) and purified by reverse phase HPLC (eluting from 5 to 95% acetonitrile in water using 0.1% TFA on a C18 column (25 mm×150 mm)) to give the product (6 mg) as a white solid in 14% yield. LCMS, ESI-MS: m/z 550 [M+H]

### Example 35

### ((1,3,4-thiadiazol-2-yl)methyl)triphenylphosphonium bromide

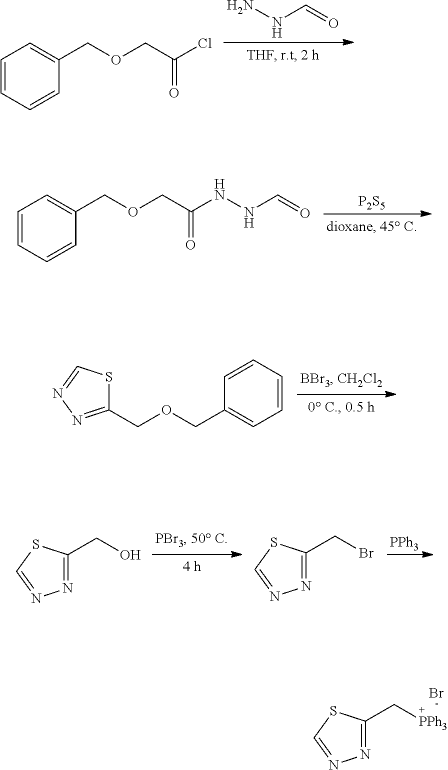

Step 1: Hydrazinecarboxaldehyde (750 mg, 12.5 mol) was added to a solution of 2-(benzyloxy)acetyl chloride (47.3 mg, 0.257 mmol) in THF (10 mL) stirred under N₂ at rt. The reaction mixture was stirred at rt for h and the solvent was evaporated. The residue was stirred in H₂O (10 mL) and CH₂Cl₂/CH₃OH (15 mL, 95:5 v/v). The organic layer was separated, washed with 1N HCl (5 ml), dried (MgSO₄), filtered and the solvent was evaporated under reduced pressure. The residue was then co-evaporated with toluene to give the pure product (26 mg) as a white solid in 45% yield. LCMS, ESI-MS: 208 [M+H].

Step 2: Phosphorous pentasulfide (0.28 g, 1.28 mmol) was added to a solution of 2-(benzyloxy)-N′-formylacetohydrazide (0.26 g, 1.28 mmol) in dioxane (13 mL) and stirred overnight at 45° C. The reaction mixture was diluted with ethyl acetate (15 mL) and washed with 1 N aq. sodium hydroxide (2×10 mL), water (3×10 mL), and brine (2×10 mL). The organic phase was dried over magnesium sulfate and evaporated under reduced pressure to give an oil product which is crystallized from methanol/water (1:4 v/v) to afford the pure product (360 mg) as a white solid in 70% yield. LCMS, ESI-MS: 206 [M+H].

Step 3: To a solution of 2-(benzyloxymethyl)-1,3,4-thiadiazole (5.75 g, 27.9 mmol) in DCM (120 mL) was added a solution of BBr₃ (1 M in DCM, 42 mL, 42 mmol) over 15 min with stirring at 0° C. After an additional 15 min at 0° C., the reaction was poured into ½ satd. Aq. NaHCO₃ (500 mL). The mixture was extracted with Et₂O (250 mL) and EtOAc (300 mL). The combined organic extracts were washed with brine (250 mL), dried over Na₂SO₄, filtered, and concentrate under reduced pressure to afford a white solid. The crude product was triturated with Et₂O/hexanes (1:9 v/v, 20 mL) causing the desired compound (0.98 g) to precipitate out as a white solid in 30% yield. LCMS, ESI-MS: 117 [M+H].

Step 4: PBr₃ (5 mL, 0.05 mmol) was added to (1,3,4-thiadiazol-2-yl)methanol (1.16 g, 10 mmol) in DCM (100 ml) with stirring at 45° C. After 4 h., water was added basifying with 0.5 N aq. sodium hydroxide solution to pH=8 and the mixture was extracted with EtOAc (2×200 mL), dried over Na₂SO₄, filtered, and concentrated under reduced pressure to give the pure product (885 mg) as a white solid in 50% yield. LCMS, ESI-MS: 179 [M+H].

Step 5: To a stirred mixture of 2-(bromomethyl)-1,3,4-thiadiazole (178 mg, 1 mmol) in DCM (10 mL) at rt, was added PPh₃ (262 mg, 1 mmol). The reaction mixture was stirred at 40° C. for 2 to 3 h. Progress of the reaction was monitored by TLC (eluting solvent: hexanes:ethyl acetate, 2:1 v/v). After reaching completion, the reaction was concentrated under reduced pressure and the residue was purified by silica gel column chromatography (eluting solvent: CH₃OH:DCM, 1:10 v/v) to obtain the pure product (210 mg) as a white solid in 50% yield. LCMS, ESI-MS: 361 [M−Br]

### Example 36

### (6R,7S)-tert-butyl 7-(tert-butoxycarbonylamino)-3-formyl-8-oxo-1-aza-bicyclo[4.2.0]oct-2-ene-2-carboxylate

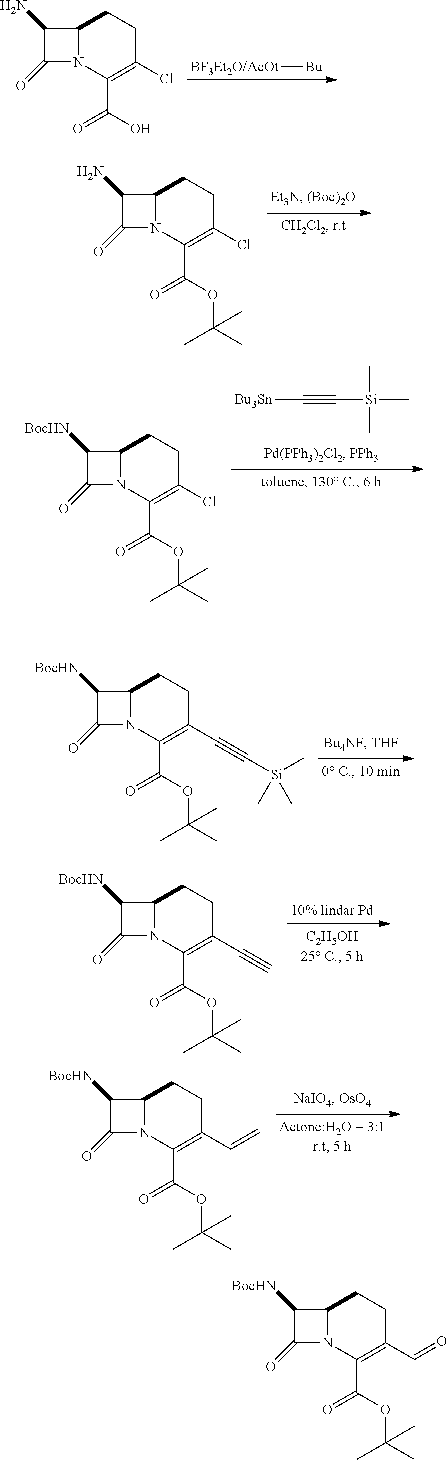

Step 1: Boron trifluoride etherate (1.2 mL, 9.06 mmol) was added to (6R,7S)-7-amino-3-chloro-8-oxo-1-aza-bicyclo[4.2.0]oct-2-ene-2-carboxylic acid (0.5 g, 1.5 mmol) in tert-butyl acetate (8.2 mL, 60.5 mmol) with stirring at 0° C. The reaction mixture was allowed to warm to rt (17° C.) and stirred for 3 h. The mixture was poured into ice and the aqueous layer was washed with MTBE (10 mL), basified with 0.5 N aq. sodium hydroxide solution (10 ml) to pH=8 keeping the temperature below 10° C. and the product was extracted with chloroform (3×15 mL). The combined organic layers were washed with brine (10 mL), dried over sodium sulfate, filtered and evaporated under reduced pressure to give pure product (0.41 g) as a white solid in 99% yield. LCMS, ESI-MS: 295 [M+Na].

Step 2: To a solution of (6R,7S)-tert-butyl-7-amino-3-chloro-8-oxo-1-aza-bicyclo[4.2.0]oct-2-ene-2-carboxylate (2.72 g, 10 mmol) in CH₂Cl₂ (20 mL), was added (Boc)₂O (4.36 g, 20 mmol) and Et₃N (1.11 g, 11 mmol) with stirring at rt. After 4 h., The reaction was washed by H₂O (5 mL), the aqueous layer was extracted with ethyl acetate (2×5 mL), the organic layers were dried over sodium sulfate, filtered and concentrated under reduced pressure. The residue was purified by silica gel column chromatography (eluting solvent: hexanes:ethyl acetate, 5:1 v/v) to obtain the pure product (2.97 g) as a white solid 80% yield. LCMS, ESI-MS: 395 [M+Na].

Step 3: Pd(PPh₃)₂Cl₂ (1.4 g, 2 mmol) was added to a solution of (6R,7S)-tert-butyl-7-(tert-butoxycarbonylamino)-3-chloro-8-oxo-1-aza-bicyclo[4.2.0]oct-2-ene-2-carboxylate (7.5 g, 20 mmol), Bu₃SnC₂Si(CH₃)₃ (23.2 g, 60 mmol) and PPh₃ (524 mg, 2 mmol) in toluene (50 mL) under nitrogen with stirring. The mixture was heated to 130° C. for 6 h and then cooled and filtered. The filtrate was concentrated under reduced pressure and the residue was purified by silica gel column chromatography (eluting solvent: hexanes:ethyl acetate, 5:1 v/v) to give the pure product (3.96 g) as a white solid in 58% yield. LCMS, ESI-MS: 457 [M+Na].

Step 4: Bu₄NF (1.8 g, 7.65 mmol) was added to a solution of (6R,7S)-tert-butyl-7-(tert-butoxycarbonylamino)-8-oxo-3-((trimethylsilyl)ethynyl)-1-aza-bicyclo[4.2.0]oct-2-ene-2-carboxylate (2 g, 5.1 mmol) in THF (50 ml) with stirring at 0° C. under nitrogen. The mixture was stirred at this temperature for 10 minutes, then portioned between EtOAc (200 ml) and 0.1 M aq. hydrochloric acid (85 mL). The EtOAc layer was dried over Na₂SO₄, filtered, concentrated, and the residue purified by silica gel column chromatography (eluting solvent hexanes:ethyl acetate, 5:1 v/v) to give the pure product (1.67 g) as a white solid in 82% yield. LCMS, ESI-MS: 385 [M+Na].

Step 5: Lindar's catalyst (120 mg) was added to the solution of (6R,7S)-tert-butyl-7-(tert-butoxycarbonylamino)-3-ethynyl-8-oxo-1aza-bicyclo[4.2.0]oct-2-ene-2-carboxylate (1000 mg, 2.76 mmol) in ethanol (100 mL) with stirring at it under hydrogen gas (balloon pressure) for 5 h. The reaction mixture was filtered, concentrated under reduced pressure to dryness to obtain product (900 mg) as a white solid in 90% yield. LCMS, ESI-MS: 387 [M+Na].

Step 6: To a solution of (6R,7S)-tert-butyl-7-(tert-butoxycarbonylamino)-8-oxo-3-vinyl-1-aza-bicyclo[4.2.0]oct-2-ene-2-carboxylate (2.1 g, 5.83 mmol) in acetone:H₂O (145 mL, 3:1 v/v), was added sodium peroxate (2.74 g, 12.8 mmol) and osmium tetroxide in H₂O (1:25 w/w, 11.8 mL). The reaction mixture was stirred for 2 h at rt, diluted with 1 M Na₂S₂SO₃ (30 mL) and extracted with ethyl acetate (3×100 mL). The organic layers were washed with water (60 mL), brine (60 mL), dried over sodium sulfate, filtered, and concentrated under reduced pressure. The residue was purified by silica gel column chromatography (eluting solvent hexanes:ethyl acetate, 5:1 v/v) to give pure product (0.84 g) as a white solid in 40% yield. LCMS, ESI-MS: 389 [M+Na], 1H-NMR (400 MHz, CDCl₃): 1.46 (s, 9H), 1.59 (s, 9H),2.05-2.16 (m, 2H), 2.90-2.96 (q, 1H), 3.89-3.93 (m, 1H), 5.09-5.11 (d, J=7.2 Hz 1H), 5.27-5.31 (t, 1H), 9.93 (s, 1H).

### Example 37

### (6R,7S)-3-((Z)-2-(1,3,4-thiadiazol-2-yl)vinyl)-7-((E)-2-(2-amino-5-chlorothiazol-4-yl)-2-(hydroxyimino)acetamido)-8-oxo-1-aza-bicyclo[4.2.0]oct-2-ene-2-carboxylic acid

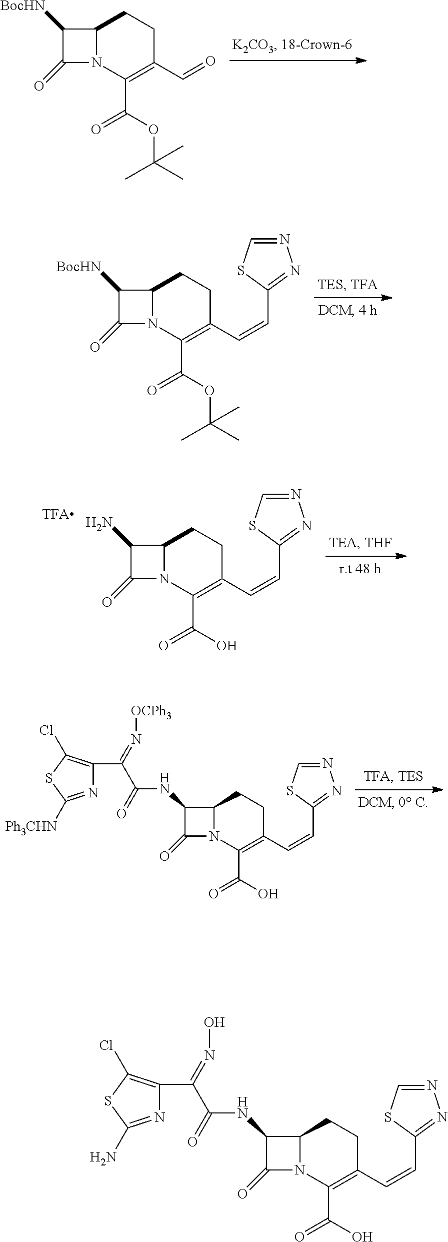

Step 1: K₂CO₃ (135 mg, 0.98 mmol) and 18-crown-6 (6 mg, 0.23 mmol) were added to a stirred solution of ((1,3,4-thiadiazol-2-yl)methyl)triphenylphosphonium bromide (418 mg, 0.98 mmol) in DCM (10 mL) with stirring. After 0.5 h., (6R,7S)-tert-butyl-7-(tert-butoxycarbonylamino)-3-formyl-8-oxo-1aza-bicyclo[4.2.0]oct-2-ene-2-carboxylate (300 mg, 0.82 mmol) was added. After stirring an additional 5 h., the mixture was partitioned between DCM (25 mL) and H₂O (10 mL). The organic phase was washed with H₂O (10 mL), dried over MgSO₄, filtrated, evaporated. The residue was purified by silica gel column chromatography (eluting solvent hexanes:ethyl acetate, 5:1 v/v) to afford (110 mg) ((6R,7S)-tert-butyl-3-((Z)-((R)-1′-(allyloxycarbonyl)-2-oxo-1,3′-bipyrrolidin-3-ylidene)methyl)-7-(tert-butoxycarbonylamino)-8-oxo-1-aza-bicyclo[4.2.0]oct-2-ene-2-carboxylate as a white solid in 20% yield. LCMS, ESI-MS: 449 [M+H].

Step 2: (6R,7S,Z)-3-(2-(1,3,4-thiadiazol-2-yl)vinyl)-7-amino-8-oxo-1-aza-bicyclo[4.2.0]oct-2-ene-2-carboxylic acid was prepared from (6R,7S,Z)-tert-butyl 3-(2-(1,3,4-thiadiazol-2-yl)vinyl)-7-(tert-butoxycarbonylamino)-8-oxo-1-aza-bicyclo[4.2.0]oct-2-ene-2-carboxylate (100 mg, 0.22 mmol) following Method A. The resulting product (58 mg) was obtained as a white powder in 90% yield and used without further purification. LCMS, ESI-MS: 293 [M+H].

Step 3: (6R,78)-3-((Z)-2-(1,3,4-thiadiazol-2-yl)vinyl)-7-((E)-2-(5-chloro-2-(tritylamino)thiazol-4-yl)-2-(trityloxyimino)acetamido)-8-oxo-1-aza-bicyclo[4.2.0]oct-2-ene-2-carboxylic acid was prepared from (6R,7S,Z)-3-(2-(1,3,4-thiadiazol-2-yl)vinyl)-7-amino-8-oxo-1-aza-bicyclo[4.2.0]oct-2-ene-2-carboxylic acid (50 mg, 0.17 mmol) following Method B. The resulting product (150 mg) was obtained as a white powder in 90% yield and used without further purification. LCMS, ESI-MS: 980 [M+H]

Step 4: (6R,7S)-3-((Z)-2-(1,3,4-thiadiazol-2-yl)vinyl)-7-((E)-2-(2-amino-5-chlorothiazol-4-yl)-2-(hydroxyimino)acetamido)-8-oxo-1-aza-bicyclo[4.2.0]oct-2-ene-2-carboxylic acid was prepared from (6R,7S)-3-((Z)-2-(1,3,4-thiadiazol-2-yl)vinyl)-7-((E)-2-(5-chloro-2-(tritylamino)thiazol-4-yl)-2-(trityloxyimino)acetamido)-8-oxo-1-aza-bicyclo[4.2.0]oct-2-ene-2-carboxylic acid (120 mg, 0.12 mmol) following Method E. The resulting product (30 mg) obtained as a white powder in 50% yield was used without further purification. LCMS, ESI-MS: 496 [M+H].

### Example 38

### MIC Assay Protocol

It is well-established that the effectiveness of β-lactam antibiotics is correlated to the amount of time that the concentration of free (unbound) drug exceeds the MIC. A serum protein binding value of >97% is considered too high for a sufficient free drug concentration to be established in a patient using any practical dosing regime. Furthermore, a compound displaying human serum binding of 70% has ten times the amount of free drug as a compound with 97% serum binding (30% vs 3%).

It will be appreciated that, in any given series of compounds, a spectrum of biological activity will be observed. In its most preferred embodiment, a compound of this invention will demonstrate activity superior to vancomycin or cefotaxime against bacterial infections resistant to conventional β-lactam antibiotics such as methicillin and ampicillin. The following procedures may, without limitation, be used to evaluate the compounds of this invention.

The in vitro MIC for bacterial isolates may be obtained in the following manner: a test compound is incorporated into a series of two-fold dilutions in cation adjusted Mueller-Hinton broth (CAMHB). Different bacterial strains diluted to provide a uniform inoculum are added to the CAMHB containing test compounds. A well without test compound is included for each strain as a growth control. The MIC is defined as the concentration of compound that completely inhibits growth as observed by the naked eye. The procedures used in these experiments are generally those standardized by the Clinical and Laboratory Standards Institute (CLSI), as set forth in the CLSI publication entitled “M7-A7. Methods for dilution antimicrobial susceptibility tests for bacteria that grow aerobically; approved standard-seventh edition.” (2006), which is incorporated by reference as if fully set forth herein. The following exemplifies such a procedure although it is to be understood that modifications of the procedure may be implemented as required.

Two-fold dilutions of the test compounds are prepared in CAMHB at 2× the concentration range to be tested if the compounds are soluble in aqueous solution. Alternately, dimethylsulfoxide (DMSO) is used to prepare two-fold dilutions at 10× the concentration range to be tested. Reference drugs such as cefotaxime, vancomycin or imipenem are used as positive controls. A few isolated colonies are retrieved from a pure culture prepared on an agar plate and suspended in PBS until the turbidity of the suspension matches a 0.5 McFarland standard which is equal to approximately 10⁸ CFU/mL. This solution is further diluted in CAMHB to 10⁶ CFU/mL if compounds are diluted in CAMHB and 5×10⁵ CFU/mL if compounds are diluted in DMSO. The CAMHB plates containing the compound dilutions are combined in equal volumes with the higher density inoculum, or 10 μL of the DMSO dilutions are added to the lower density inoculum. When S. aureus is the organism being tested and the compound is oxacillin, a beta-lactam or a carbacephem compound of the present invention, 2% NaCl is added to the growth media. The plates are then incubated for 16-20 h at 35° C. The plates are then observed to determine which concentration of the test compound is the MIC.

Data for certain representative compounds is shown in Table 1 below.

TABLE 1

| | ASAU | ASAU | ASAU | ASAU | ASAU | ASAU |
|---|---|---|---|---|---|---|
| Example # | 001 | 1028 | 1029 | 002 | 1043 | 1033 |
| Comparative | A | A | A | B | B | B |
| Compound 1 | | | | | | |
| Comparative | A | B | B | A | B | B |
| Compound 2 | | | | | | |
| 17 | A | B | B | C | C | C |
| 18 | A | A | A | A | B | B |
| 23 | A | B | B | B | C | C |
| 25 | A | B | B | B | C | C |
| 27 | A | B | B | B | C | C |
| 29 | A | A | A | B | B | B |
| 31 | A | B | B | B | B | B |
| 34 | A | B | A | B | B | B |

| | | |
|---|---|---|
| * Key: | | |
| Strain | ACH Code | Phenotype |
| S. aureus | ASAU001 | Susceptible Organism ATCC 29213 |
| | ASAU1028 | CA-MRSA, USA400 |
| | ASAU1029 | CA-MRSA, USA300 |
| | ASAU002 | MRSA ATCC 33591 |
| | ASAU1043 | MRSA, CIP, ERY, CLI, GEN resistant |
| | ASAU1033 | MRSA, CIP, ERY, CLIN, TET, GEN |
| | | resistant |

** MIC Key:
MIC's of 1.0 μg/mL or less = A
MIC's of greater than 1.0 μg/mL to 8.0 μg/mL = B
MIC's of greater than 8.0 μg/mL = C
*** Comparative Compounds:
Comparative Compound 1 is:

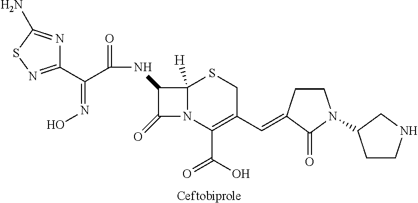

Comparative Compound 2 is:

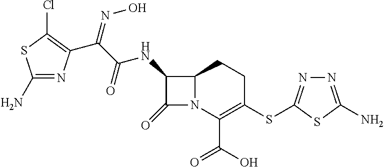

### Example 39

Compounds that show superior activity in in vitro tests can then be further evaluated in animal models such as rats and mice. The following is an example of such a test, it being understood that the example is not to be construed as limiting the scope of this invention in any manner whatsoever.

Staphylococcus aureus strain Smith (ATCC 13709, penicillin-susceptible) or strain 76 (methicillin-resistant) is grown overnight at 37° C. in brain-heart infusion broth (BHIB). The following morning, it is sub-cultured to fresh BHIB and incubated for 4-5 h at 37° C. The cells are harvested by centrifugation, washed twice with PBS, and adjusted to the desired inoculum. The cell suspension is then mixed with an equal volume of sterile 14% hog-gastric mucin (Comber K. R., et al., Antimicrobial Agents and Chemotherapy, 1995, 7(2):179-185). The inoculum is kept in an ice bath until ready for use (preferably less than one hour).

Male Swiss-Webster mice are challenged intraperitoneally with 0.5 mL of the above bacterial suspension of S. aureus strain Smith (LD₅₀). Test compounds are administered subcutaneously in 0.1 mL volumes immediately after inoculation and again 2 h later. The animals are then observed for 72 h. The total dose associated with 50% survival (ED₅₀) is then determined using the probit method (Pasiello, A. P., et al., J. Toxicol. Environ. Health, 1977, 3:797 809).

As noted previously, to be an effective anti-MRSA compound, a carbacephem must exhibit a proper balance of potency versus serum protein binding. The following procedure may be used to evaluate serum binding: compounds are incubated in serum for 10 min at 37° C. in a shaking water bath. Then a serum ultrafiltrate is obtained by centrifugation of ultra-filtration units (Amicon Centrifree) for, say, 20 minutes at 25° C. Compound content in the ultrafiltrate is quantified by HPLC using standards prepared in blank ultra-filtrate undergoing similar processing.

The range of utility of the compounds herein can easily be established by those skilled in the art using the disclosures herein and all bacteria within the useful range are within the scope of this invention.

All of the U.S. patents, U.S. patent application publications, U.S. patent applications, foreign patents, foreign patent applications and non-patent publications referred to in this specification are incorporated herein by reference in their entirety to the extent not inconsistent with the present description.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A compound having the following structure (I): 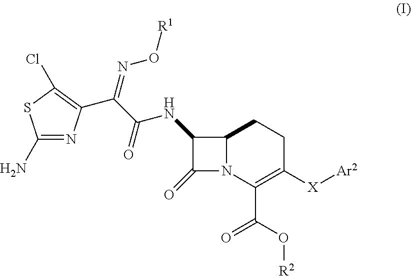
or a stereoisomer, pharmaceutically acceptable salt, ester, or prodrug thereof,
wherein:
R¹ is selected from hydrogen, optionally substituted alkyl, optionally substituted alkenyl, optionally substituted alkynyl, optionally substituted alkoxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl and —C(═O)R^{1a},
wherein:
R^{1a} is selected from hydrogen, optionally substituted alkyl, optionally substituted alkenyl, optionally substituted alkynyl, optionally substituted alkoxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl and optionally substituted heteroarylalkyl;
R² is selected from hydrogen, optionally substituted alkyl, optionally substituted alkenyl, optionally substituted alkynyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl and optionally substituted heteroarylalkyl;
X is selected from —O—, —C(═O)—, —SCH₂—, —CH₂S—, —SCH═CH—, —CH═CHS—, —SCH₂S—, —OCH₂—, —CH₂O—, optionally substituted alkylene, optionally substituted alkenylene and optionally substituted cycloalkyl; and
Ar² is optionally substituted aryl or optionally substituted heteroaryl.

2. A compound of claim 1 wherein R¹ is hydrogen.

3. A compound of claim 1 wherein R¹ is alkyl and is selected from methyl, ethyl, n-propyl, iso-propyl, n-butyl, tert-butyl, iso-butyl and sec-butyl.

4. A compound of claim 1 wherein R¹ is substituted alkyl and is optionally substituted haloalkyl.

5-8. (canceled)

9. A compound of claim 1 wherein R¹ is alkenyl and is —CH₂CH═CH₂.

10-11. (canceled)

12. A compound of claim 1 wherein R¹ is cycloalkyl and is selected from cyclopropyl, cyclobutyl, cyclopentyl, cyclopentenyl, cyclohexyl, and cyclohexenyl.

13. A compound of claim 1 wherein R² is hydrogen.

14. A compound of claim 1 wherein R² is alkyl and is selected from methyl, ethyl, n-propyl, iso-propyl, n-butyl, tent-butyl, iso-butyl and sec-butyl.

15-16. (canceled)

17. A compound of claim 1 wherein R² is cycloalkyl and is selected from cyclopropyl, cyclobutyl, cyclopentyl and cyclohexyl.

18-21. (canceled)

22. A compound of claim 1 wherein the compound is a prodrug of structure (I) having the following structure (III): 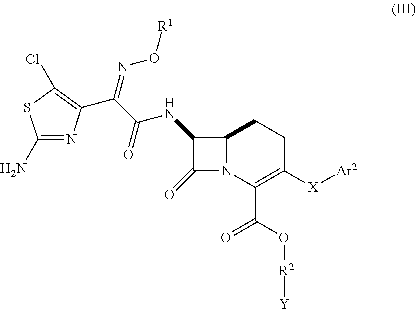
wherein R² and Y, taken together, are selected from:

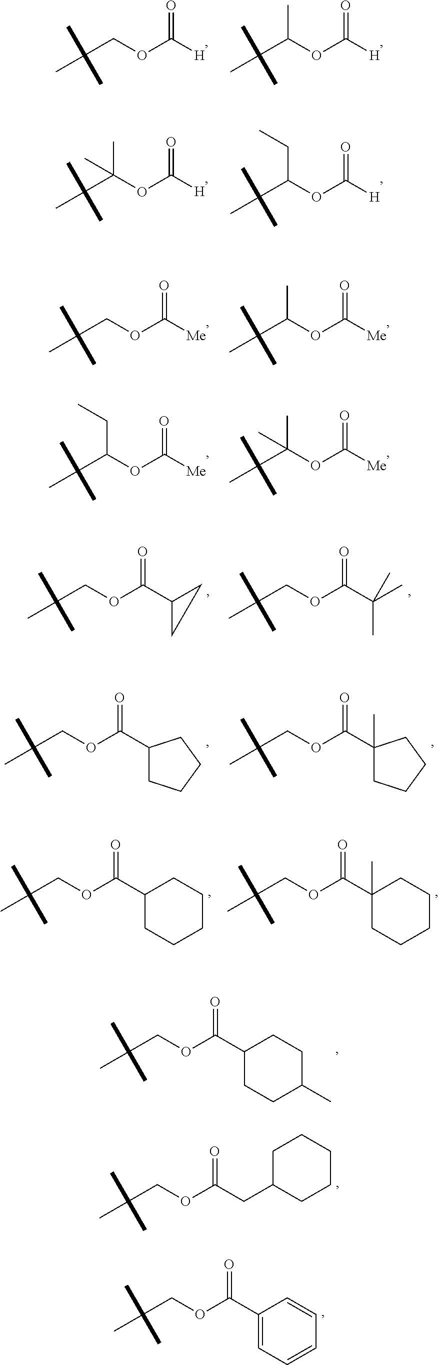

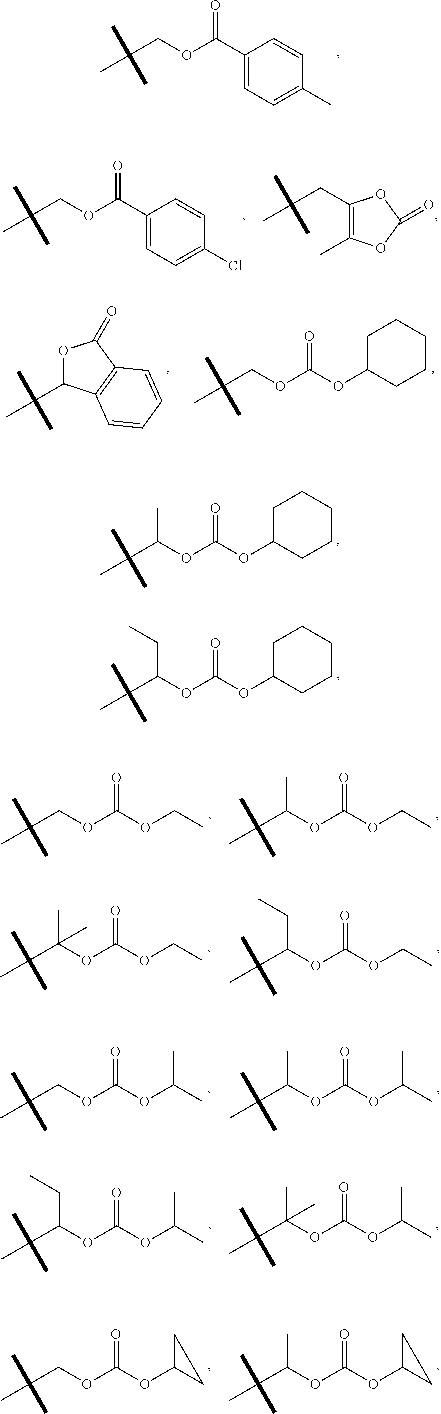

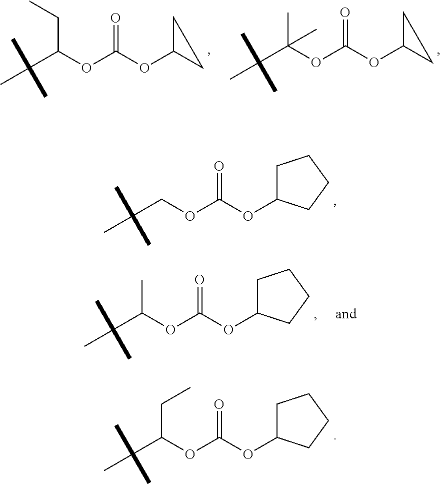

23. (canceled)

24. A compound of claim 1 wherein X is —SCH₂— or —CH₂S—.

25. A compound of claim 1 wherein X is —SCH₂S—.

26-33. (canceled)

34. A compound of claim 1 any one of claim 1 wherein X is alkenylene.

35-43. (canceled)

44. A pharmaceutical composition comprising a compound of claim 1, or a stereoisomer, pharmaceutically acceptable salt or prodrug thereof, and a pharmaceutically acceptable carrier, diluent or excipient.

45. A method of treating a bacterial infection in a mammal in need thereof, comprising administering to the mammal an effective amount of a compound of claim 1.

46. The method of claim 45 wherein the bacterial infection is caused by a β-lactam antibiotic-resistant bacterium.

47. (canceled)

48. A compound having the following structure: 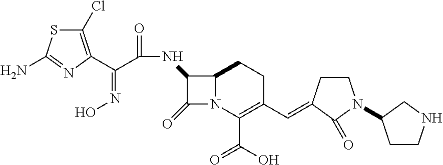
or a stereoisomer, pharmaceutically acceptable salt, ester, or prodrug thereof.

49. A pharmaceutical composition comprising the compound of claim 48, or a stereoisomer, pharmaceutically acceptable salt or prodrug thereof, and a pharmaceutically acceptable carrier, diluent or excipient.

50. A method of treating a bacterial infection in a mammal in need thereof, comprising administering to the mammal an effective amount of the compound of claim 48.

51. The method of claim 50 wherein the bacterial infection is caused by a β-lactam antibiotic-resistant bacterium.

52. (canceled)
